(19) United States
(12) Patent Application Publication
(10) Pub. No.: US 2026/0022197 A1
(43) Pub. Date: Jan. 22, 2026

(54) METHODS OF REVERSING TICAGRELOR ACTIVITY

(71) Applicant: SFJ Pharma X, Inc., Pleasanton, CA (US)

(72) Inventor: Barbara A. WHITE, Towson, MD (US)

(21) Appl. No.: 19/270,367

(22) Filed: Jul. 15, 2025

Related U.S. Application Data
(60) Provisional application No. 63/672,219, filed on Jul. 16, 2024.

(51) Int. Cl. C07K 16/44; A61P 7/04

(52) U.S. Cl. CPC C07K 16/44; A61P 7/04

(57) ABSTRACT

Herein is provided a method of reversing ticagrelor-enhanced bleeding in a patient comprising administering to said patient during surgery, in a perioperative setting, or during an invasive procedure an antibody or antigen-binding fragment thereof that binds to ticagrelor. Also provided is a method of reversing ticagrelor-enhanced bleeding in a patient comprising administering an antibody or antigen-binding fragment thereof that binds to ticagrelor, wherein the patient is elderly, has underlying cardiac or pulmonary or metabolic disease, optionally wherein the patient has a minimum percent inhibition of P2Y₁₂ reaction units (PRU) over 4 hours after starting administration of the antibody or antigen-binding fragment thereof that is less than the percent inhibition of PRU before administration.

### CROSS-REFERENCE TO RELATED APPLICATIONS

This application claims priority benefit of U.S. Provisional Patent Application Ser. No. 63/672,219, filed on Jul. 16, 2024, the entire contents of which are incorporated herein by reference.

### REFERENCE TO AN ELECTRONIC SEQUENCE LISTING

The contents of the electronic sequence listing (307602000400SEQLIST.xml; Size: 8,200 bytes; and Date of Creation: Jul. 7, 2025) is herein incorporated by reference in its entirety.

### FIELD OF INVENTION

Provided herein are methods for reversing ticagrelor-enhanced bleeding in a patient comprising administering bentracimab.

### BACKGROUND OF THE INVENTION

Ticagrelor is an orally available inhibitor of platelet activity that in combination with low-dose acetylsalicylic acid (ASA; aspirin) has been shown to prevent recurrent thrombotic events, e.g., myocardial infarction (MI), stroke, and cardiovascular death, in patients with acute coronary syndrome (ACS) or a history of MI. Ticagrelor also reduces the rate of stent thrombosis in patients who have been stented for treatment of ACS.

Ticagrelor is a direct-acting cyclopentyltriazolopyrimidine inhibitor of platelet function that selectively and reversibly binds and antagonizes the platelet P2Y₁₂ receptor thereby preventing adenosine diphosphate (ADP) from causing platelet aggregation (Teng, R. et al. (2016). Journal of Thrombosis and Haemostasis, 14(12), 2342-2352; Van Giezen, J. J. J., et al. (2009). Journal of thrombosis and haemostasis, 7(9), 1556-1565.). A ticagrelor active metabolite (TAM) achieves a 30% to 40% plasma exposure relative to the circulating ticagrelor exposure in humans, (Storey, R. H. (2007). JACC, 50(19), 1852-6), and has potency similar to ticagrelor in inhibiting the P2Y₁₂ receptor. Ticagrelor is considered the best-in-class P2Y₁₂ antiplatelet agent because it has demonstrated superior efficacy compared to clopidogrel. Yet, the side-effects of ticagrelor treatment can cause spontaneous bleeding. Further, due to its anti-platelet activity, ticagrelor treatment can be dangerous in patients requiring emergency surgery, and leads to increased blood loss. P2Y₁₂ reaction units (PRU) is a measure of platelet aggregation. PRU is inhibited in patients taking ticagrelor.

Ticagrelor is administered in combination with low-dose ASA as part of a regimen called dual antiplatelet therapy (DAPT). Although DAFT is strongly recommended in the management of patients experiencing an ACS event, it is also known to increase the risk of major or life-threating bleeding. Storey, R. F. (2011). Heart, 97(15), 1262-1267. In the event of major bleeding in a patient on DAFT, there are limited treatment options. There are no approved drugs or biological agents capable of reversing the P2Y₁₂ inhibition produced by ticagrelor or other P2Y₁₂ inhibitors.

Bentracimab, also known as PB2452, is an intravenously delivered monoclonal antibody fragment (Fab) that binds to ticagrelor and intended to reverse its antiplatelet effects. The transient binding of ticagrelor to the P2Y₁₂ receptor allows anti-ticagrelor agents, such as bentracimab, a human Fab fragment that binds to ticagrelor, to bind to free ticagrelor, thereby preventing ticagrelor's activation of the receptor and removing ticagrelor from circulation. With ticagrelor bound to bentracimab or removed, ADP can once again bind the P2Y₁₂ receptor and induce platelet aggregation. Bentracimab also binds to the major active metabolite of ticagrelor, AR-C124910XX (TAM).

Currently, there are no approved effective therapy to mitigate ticagrelor-induced platelet inhibition in patients, which represents a significant unmet need.

### SUMMARY OF THE INVENTION

Provided herein are methods of reversing ticagrelor-enhanced bleeding in a patient comprising administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient during surgery or during an invasive procedure, wherein the antibody or antigen-binding fragment thereof comprises a heavy chain complementarity determining region (CDRH)1, a CDRH2, and a CDRH3 of a variable heavy chain region (VH) comprising the amino acid sequence set forth in SEQ ID NO:01 and a light chain complementarity determining region (CDRL)1, CDRL2, and a CDRL3 of a variable light chain region (VL) comprising the amino acid sequence set forth in SEQ ID NO:02. In any of the proceeding embodiments, the antibody or antigen-binding fragment thereof is administered during surgery. In some embodiments, the surgery is associated with a risk of significant bleeding. In some embodiments, the surgery is cardiac surgery, gastrointestinal surgery, vascular surgery, thoracic surgery, neurosurgery, urogenital surgery, or major orthopedic surgery. In some embodiments, the surgery has an adverse procedural outcome if hemostasis is impaired. In some embodiments, the surgery is coronary artery bypass graft (CABG), cardiac surgery, gastrointestinal surgery, coronary artery procedure, repair of aortic dissection, and/or cardiac valve replacement. In some embodiments, the surgery is CABG.

In any of the proceeding embodiments, administering the antibody or antigen-binding fragment thereof to the patient results in a minimum percent inhibition of P2Y₁₂ reaction units (PRU) over 4 hours after starting administration of between about −78 and about −56 in the patient.

In any of the proceeding embodiments, administering the antibody or antigen-binding fragment thereof to a population of patients results in a mean minimum percent inhibition of PRU over 4 hours after starting administration of between about −78 and about −56 in the population of patients.

In some embodiments, the patient does not receive treatment with the antibody or antigen-binding fragment thereof prior to start of surgery.

In some embodiments, the antibody or antigen-binding fragment thereof is administered in a perioperative setting.

In some embodiments, the antibody or antigen-binding fragment thereof is administered during an invasive procedure. In some embodiments, the invasive procedure is percutaneous cardiac intervention or another cardiac or vascular procedure. In some embodiments, the invasive procedure is a cardiac valve replacement.

Provided herein are methods of reversing ticagrelor-enhanced bleeding in a patient comprising administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient, wherein the antibody or antigen-binding fragment thereof comprises a CDRH1, a CDRH2, and a CDRH3 of a VH comprising the amino acid sequence set forth in SEQ ID NO:01 and a CDRL1, CDRL2, and a CDRL3 of a VL comprising the amino acid sequence set forth in SEQ ID NO:02, wherein the patient has metabolic disease. In some embodiments, the patient is elderly and/or has underlying cardiac or pulmonary disease and/or has limited cardiac reserve.

Provided herein are methods of reversing ticagrelor-enhanced bleeding in a patient comprising administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient, wherein the antibody or antigen-binding fragment thereof comprises a CDRH1, a CDRH2, and a CDRH3 of a VH comprising the amino acid sequence set forth in SEQ ID NO:01 and a CDRL1, CDRL2, and a CDRL3 of a VL comprising the amino acid sequence set forth in SEQ ID NO:02, wherein the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, percent inhibition of PRU is determined by calculating 100×[(180−PRU)/180].

Provided herein are methods of reversing ticagrelor-enhanced bleeding in a patient comprising: i) administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient, wherein the antibody or antigen-binding fragment thereof comprises a CDRH1, a CDRH2, and a CDRH3 of a VH comprising the amino acid sequence set forth in SEQ ID NO:01 and a CDRL1, CDRL2, and a CDRL3 of a VL comprising the amino acid sequence set forth in SEQ ID NO:02, and ii) improving percent inhibition of PRU in the patient.

Provided herein are methods of reversing ticagrelor-enhanced bleeding in a patient comprising administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient, wherein the antibody or antigen-binding fragment thereof comprises a CDRH1, a CDRH2, and a CDRH3 of a VH comprising the amino acid sequence set forth in SEQ ID NO:01 and a CDRL1, CDRL2, and a CDRL3 of a VL comprising the amino acid sequence set forth in SEQ ID NO:02, wherein administration of the antibody or antigen-binding fragment thereof to a population of patients results in a treatment-emergent adverse event related to the administration of the antibody or antigen-binding fragment thereof in less than 3% of patients within the population of patients.

In any of the proceeding embodiments, the antibody or antigen-binding fragment thereof comprises CDR combinations comprising SEQ ID NO:03 (CDRH1), SEQ ID NO:04 (CDRH2), SEQ ID NO:05 (CDRH3), SEQ ID NO:06 (CDRL1), SEQ ID NO:07 (CDRL2), and SEQ ID NO:08 (CDRL3). In any of the proceeding embodiments, the VH has an amino acid sequence according to SEQ ID NO:01 and the VL has an amino acid sequence according to SEQ ID NO:02. In any of the proceeding embodiments, the antibody or antigen-binding fragment is an antibody. In any of the proceeding embodiments, the antibody or antigen-binding fragment is an antigen-binding fragment. In some embodiments, the antigen-binding fragment is a Fab.

In any of the proceeding embodiments, the antibody or antigen-binding fragment thereof is administered within about one day of the last dose of ticagrelor. In some embodiments, the antibody or antigen-binding fragment thereof is administered on the same day as the most recent dose of ticagrelor.

In any of the proceeding embodiments, the patient was previously treated with a daily dose of between about 60 mg and about 360 mg of ticagrelor. In any of the proceeding embodiments, the patient was previously treated with ticagrelor for about 1 to 12 months. In any of the proceeding embodiments, the patient was previously treated with ticagrelor for about 0 to 1 month. In any of the proceeding embodiments, the patient was previously treated with ticagrelor for about 1 to 10 years.

In any of the proceeding embodiments, prior to administration of the antibody or antigen-binding fragment thereof, the patient has PRU of between about 180 to about 230.

In any of the proceeding embodiments, PRU is not measured before administering the antibody or antigen-binding fragment thereof.

In any of the proceeding embodiments, the patient is in need of gastrointestinal surgery, vascular surgery, thoracic surgery, or urogenital surgery.

In any of the proceeding embodiments, the dose of acetylsalicylic acid is 0 mg to 100 mg. In any of the proceeding embodiments, the patient receives chronic treatment with acetylsalicylic acid.

In any of the proceeding embodiments, administering the antibody or antigen-binding fragment thereof to the patient results in an increase in PRU in the patient. In some embodiments, administering the antibody or antigen-binding fragment thereof to the patient results in the patient having a minimum percent inhibition of PRU over 4 hours after starting administration of between about −76 and about −67, wherein the patient requires urgent surgery or has major bleeding. In some embodiments, administering the antibody or antigen-binding fragment thereof to a population of patients results in a mean minimum percent inhibition of PRU over 4 hours after starting administration of between about −76 and about −67 in the population of patients, wherein the population of patients requires urgent surgery or has major bleeding. In some embodiments, administering the antibody or antigen-binding fragment thereof to the population of patients with major bleeding or requiring urgent surgery results in a difference of about −129 to −115 between the minimum percent inhibition of PRU over 4 hours after starting administration and the percent inhibition of PRU before administration in the patient, wherein the minimum percent inhibition of P2Y₁₂ reaction units (PRU) over 4 hours after starting administration of the antibody or antigen-binding fragment thereof is less than the percent inhibition of PRU before administration. In some embodiments, administering the antibody or antigen-binding fragment thereof results in the patient with major bleeding or requiring urgent surgery achieving good or excellent homeostasis. In some embodiments, administering the antibody or antigen-binding fragment thereof to the population of patients results in excellent or good hemostasis in about 95% of the population of patients.

In some embodiments, administering the antibody or antigen-binding fragment thereof to the patient results in a minimum percent inhibition of P2Y₁₂ reaction units (PRU) over 4 hours after starting administration of between about −78 and about −56 in the patient, wherein the patient has uncontrolled major or life-threatening bleeding. In some embodiments, administering the antibody or antigen-binding fragment thereof to a population of patients with uncontrolled major or life-threatening bleeding results in a mean minimum percent inhibition of PRU over 4 hours after starting administration of between about −78 and about −56 in the population of patients. In some embodiments, administering the antibody or antigen-binding fragment thereof to the population of patients with uncontrolled major or life-threatening bleeding results in a difference of about −130 to −101 between the minimum percent inhibition of PRU over 4 hours after starting administration and the percent inhibition of PRU before administration in the patient, wherein the minimum percent inhibition of P2Y₁₂ reaction units (PRU) over 4 hours after starting administration of the antibody or antigen-binding fragment thereof is less than the percent inhibition of PRU before administration. In some embodiments, administering the antibody or antigen-binding fragment thereof to the patient with uncontrolled major or life-threatening bleeding results in the patient achieving excellent or good hemostasis. In some embodiments, administering the antibody or antigen-binding fragment thereof to the population of patients with uncontrolled major or life-threatening bleeding results in excellent or good hemostasis in about 80% of the population of patients.

In some embodiments, administering the antibody or antigen-binding fragment thereof to the patient results in a minimum percent inhibition of P2Y₁₂ reaction units (PRU) over 4 hours after starting administration of between about −78 and about −68 in the patient, wherein the patient requires surgery or invasive procedure. In some embodiments, administering the antibody or antigen-binding fragment thereof to a population of patients requiring surgery or invasive procedure results in a mean minimum percent inhibition of PRU over 4 hours after starting administration of between about −78 and about −68 in the population of patients. In some embodiments, administering the antibody or antigen-binding fragment thereof to the population of patients requiring surgery or invasive procedure results in a difference of about −132 to −117 between the minimum percent inhibition of PRU over 4 hours after starting administration and the percent inhibition of PRU before administration in the patient, wherein the minimum percent inhibition of P2Y₁₂ reaction units (PRU) over 4 hours after starting administration of the antibody or antigen-binding fragment thereof is less than the percent inhibition of PRU before administration. In some embodiments, administering the antibody or antigen-binding fragment thereof to the patient requiring surgery or invasive procedure results in the patient achieving excellent or good hemostasis. In some embodiments, administering the antibody or antigen-binding fragment thereof to a population of patients requiring surgery or invasive procedure results in excellent or good hemostasis in about 100% of the population of patients.

In any of the proceeding embodiments, the method further comprises resuming ticagrelor treatment after administering the antibody or antigen-binding fragment thereof, wherein treatment with ticagrelor is restarted 8 hours to 3 days after administering the antibody or antigen-binding fragment thereof.

In any of the proceeding embodiments, the antibody or antigen-binding fragment thereof is administered to the patient intravenously.

In some embodiments, a first patient who is administered the antibody or antigen-binding fragment thereof after receiving ticagrelor has a lower risk of severe bleeding compared to a second patient who is not administered the antibody or antigen-binding fragment thereof after receiving ticagrelor, wherein the first patient and the second patient both require urgent surgery. In some embodiments, the risk of severe bleeding of the first patient who is administered the antibody or antigen-binding fragment thereof after receiving ticagrelor is about 17.7 to about 29.0 lower than that of the second patient who is not administered the antibody or antigen-binding fragment thereof after receiving ticagrelor. In some embodiments, the risk of severe bleeding of the first patient who is administered the antibody or antigen-binding fragment thereof after receiving ticagrelor is about 23.4 lower than that of the second patient who is not administered the antibody or antigen-binding fragment thereof. In some embodiments, the urgent surgery is CABG, and wherein the risk of severe bleeding of the first patient who is administered the antibody or antigen-binding fragment thereof after receiving ticagrelor is about 14.8 to about 31.6 lower than that of the second patient who is not administered the antibody or antigen-binding fragment thereof after receiving ticagrelor. In some embodiments, the risk of severe bleeding of the first patient who is administered the antibody or antigen-binding fragment thereof after receiving ticagrelor is about 22.5 lower than that of the second patient who is not administered the antibody or antigen-binding fragment thereof after receiving ticagrelor. In some embodiments, a first population of patients requiring urgent surgery and who are administered the antibody or antigen-binding fragment thereof after receiving ticagrelor has a lower risk of severe bleeding compared to a second population of patients requiring urgent surgery and who are not administered the antibody or antigen-binding fragment thereof after receiving ticagrelor. In some embodiments, the risk of severe bleeding of the first population of patients who are administered the antibody or antigen-binding fragment thereof after receiving ticagrelor is about 17.7 to about 29.0 lower than that of the second population of patients who is not administered the antibody or antigen-binding fragment thereof after receiving ticagrelor. In some embodiments, the urgent surgery is CABG, and wherein the risk of severe bleeding of the first population of patients who are administered the antibody or antigen-binding fragment thereof after receiving ticagrelor is about 14.8 to about 31.6 lower than that of the second population of patients who are not administered the antibody or antigen-binding fragment thereof after receiving ticagrelor. In some embodiments, the risk of severe bleeding of the first population of patients who are administered the antibody or antigen-binding fragment thereof after receiving ticagrelor is about 22.5 lower than that of the second population of patients who are not administered the antibody or antigen-binding fragment thereof after receiving ticagrelor. In some embodiments, the risk is a relative risk.

In some embodiments, a first patient who is administered the antibody or antigen-binding fragment thereof after receiving ticagrelor has a lower risk of mortality compared to a second patient who is not administered the antibody or antigen-binding fragment thereof after receiving ticagrelor, wherein the first patient and the second patient both have major bleeding. In some embodiments, the major bleeding is procedure-related bleeding, non-procedure-related or spontaneous major bleeding, non-procedure-related or spontaneous intracerebral hematoma (ICH), or non-procedure-related or spontaneous non-ICH. In some embodiments, the risk of mortality of the first patient who is administered the antibody or antigen-binding fragment after receiving ticagrelor is about 1.0 to about 16.4 lower than that of the second patient who is not administered the antibody or antigen-binding fragment thereof after receiving ticagrelor. In some embodiments, the risk of mortality of the first patient who is administered the antibody or antigen-binding fragment after receiving ticagrelor is about 7.7 lower than that of the second patient who is not administered the antibody or antigen binding fragment thereof after receiving ticagrelor. In some embodiments, a first population of patients with major bleeding and who are administered the antibody or antigen-binding fragment thereof after receiving ticagrelor has a lower risk of mortality compared to a second population of patients with major bleeding and who are not administered the antibody or antigen-binding fragment thereof after receiving ticagrelor. In some embodiments, the risk of mortality of the first population of patients who is administered the antibody or antigen-binding fragment thereof after receiving ticagrelor is about 1.0% to about 16.4 lower than that of the second population of patients who is not administered the antibody or antigen-binding fragment thereof after receiving ticagrelor. In some embodiments, the antibody or antigen-binding fragment thereof is administered to the first patient during surgery or during an invasive procedure. In some embodiments, the risk is a relative risk.

In some embodiments, a first patient who is administered the antibody or antigen-binding fragment thereof after receiving ticagrelor has a lower risk of mortality compared to a second patient who is not administered the antibody or antigen-binding fragment thereof after receiving ticagrelor, wherein the first patient and the second patient both require urgent surgery. In some embodiments, the risk of mortality of the first patient who is administered the antibody or antigen-binding fragment thereof after receiving ticagrelor is at least about 1.3 to about 11.4 lower than that of a second patient who is not administered the antibody or antigen-binding fragment thereof after receiving ticagrelor. In some embodiments, the risk of mortality of the first patient who is administered the antibody or antigen-binding fragment thereof after receiving ticagrelor is about 6.6 lower than that of the second patient who is not administered the antibody or antigen-binding fragment thereof after receiving ticagrelor. In some embodiments, a first population of patients requiring urgent surgery and who are administered the antibody or antigen-binding fragment thereof after receiving ticagrelor has a lower risk of mortality compared to a second population of patients requiring urgent surgery and who are not administered the antibody or antigen-binding fragment thereof after receiving ticagrelor. In some embodiments, the risk of mortality of the first population of patients who are administered the antibody or antigen-binding fragment thereof after receiving ticagrelor is at least about 1.3 to about 11.4 lower than that of the second population of patients who are not administered the antibody or antigen-binding fragment thereof after receiving ticagrelor. In some embodiments, the risk of mortality of the first population of patients who are administered the antibody or antigen-binding fragment thereof after receiving ticagrelor is about 6.6 lower than that of the second population of patients who are not administered the antibody or antigen-binding fragment thereof after receiving ticagrelor. In some embodiments, a first patient who is administered the antibody or antigen-binding fragment thereof after receiving ticagrelor has a lower risk of mortality compared to a second patient who is not administered the antibody or antigen-binding fragment thereof after receiving ticagrelor, wherein the first patient and the second patient require urgent surgery or have major bleeding. In some embodiments, the risk of mortality of the first patient who is administered the antibody or antigen-binding fragment thereof after receiving ticagrelor is at least about 0.5 to about 9.5 lower than that of the second patient who is not administered the antibody or antigen-binding fragment thereof after receiving ticagrelor. In some embodiments, the risk of mortality of the first patient who is administered the antibody or antigen-binding fragment thereof after receiving ticagrelor is about 5.4 lower than that of the second patient who is not administered the antibody or antigen-binding fragment thereof after receiving ticagrelor. In some embodiments, the risk is a relative risk.

In some embodiments, a first population of patients who are administered the antibody or antigen-binding fragment thereof after receiving ticagrelor has a lower risk of mortality compared to a second population of patients who are not administered the antibody or antigen-binding fragment thereof after receiving ticagrelor, wherein the first population of patients and the second population of patients require urgent surgery or have major bleeding. In some embodiments, the risk of mortality of the first population of patients who are administered the antibody or antigen-binding fragment thereof after receiving ticagrelor is at least about 0.5 to about 9.5 lower than that of the second population of patients who are not administered the antibody or antigen-binding fragment thereof after receiving ticagrelor. In some embodiments, the risk of mortality of the first population of patients who are administered the antibody or antigen-binding fragment thereof after receiving ticagrelor is about 5.4 lower than that of the second population of patients who are not administered the antibody or antigen-binding fragment thereof after receiving ticagrelor. In some embodiments, the mortality is due to any cause within 38 days after start of major bleeding or within 38 days after start of urgent surgery. In some embodiments, the risk is a relative risk.

### DESCRIPTION OF THE FIGURES

FIG. 1 shows the minimum % inhibition of PRU in over 4 hours in total patient population (primary endpoint), patients requiring surgery or invasive procedure (surgery or procedure group; 2^{nd} secondary endpoint), and patients with major or life-threatening bleeding (major bleeding group; 3^{rd} secondary endpoint). These three endpoints are represented by the black bars. White bars represent % inhibition of PRU at baseline, i.e., prior to treatment with bentracimab. Minimum % inhibition of PRU was assessed by the VerifyNow™ PRUTest™ platelet function assay. The dotted line denotes normal P2Y12-mediated platelet aggregation.

FIGS. 2A-2B show the % inhibition of PRU in the total enrolled population, patients requiring surgery or invasive procedure (surgery or procedure group), and patients with major or life-threatening bleeding (major bleeding group) from pre-dose to 4 hours after starting administration of bentracimab (FIG. 2A) or from pre-dose to 72 hours after starting administration of bentracimab (FIG. 2B). The dotted line denotes normal P2Y12-mediated platelet aggregation.

FIG. 3A-3B show the PRU in the total enrolled population, patients requiring surgery or invasive procedure (surgery or procedure group), and patients with major or life-threatening bleeding (major bleeding group) from pre-dose to 4 hours after starting administration of bentracimab (FIG. 3A) or from pre-dose to 72 hours after starting administration of bentracimab (FIG. 3B). FIGS. 3C-3F show analysis of PRU within the enrolled population by age (FIG. 3C), sex (FIG. 3D), kidney disease status (FIG. 3E), or race (FIG. 3F). The dotted line denotes normal P2Y12-mediated platelet aggregation.

FIG. 4 shows a forest plot for minimum % inhibition of PRU over 4 hours for subgroups of various patient populations. CYP3A=Cytochrome P450, family 3, subfamily A; ESRD=End-stage renal failure.

FIGS. 5A-5C show Kaplan-Meier curves for first transfusion with red blood cells or whole blood after start of bentracimab infusion in Phase 3 or equivalent start in PLATO using sIPTW propensity score weighting in the overall population of patients with major bleeding (FIG. 5A), subpopulation of patients with procedure-related major bleeding, (FIG. 5B), and subpopulation of patients with spontaneous major bleeding (FIG. 5C).

### DETAILED DESCRIPTION OF THE INVENTION

The present invention is at least partly based upon the surprising efficacy of the ticagrelor-binding Fab, bentracimab, for rapidly reversing ticagrelor-enhanced bleeding in 1) patients with uncontrolled major or life-threatening bleeding; and 2) patients in need of surgery or an invasive procedure. Prior to treatment, patients taking ticagrelor experience inhibition of PRU, a measure of platelet aggregation. Treatment with bentracimab caused unexpectedly rapid reversal of ticagrelor-enhanced bleeding in both patient populations, as measured by rapid decrease in percent inhibition of PRU after starting bentracimab administration. This effect is especially surprising in patients with uncontrolled major or life-threatening bleeding, which includes patients who started receiving bentracimab during surgery, because reversal agents for anticoagulants have traditionally been used only in a preventative context, e.g., prior to start of surgery, and are not thought to act quickly enough to be used during surgery. For example, the monoclonal antibody fragment idarucizumab was reported to be effective in “rapidly, durably and safely” reversing the anticoagulant effect of dabigatran in Phase III clinical trial “[i]n emergency situations.” Yet, in this study, idarucizumab was administered to the surgery arm about 1.6 hours prior to the initiation of the intended procedure. Pollack Jr, C. V., et al. (2017). New England Journal of Medicine, 377(5), 431-441. Meanwhile, the data disclosed within the present application show surprisingly that bentracimab can effectively reverse ticagrelor-enhanced bleeding when administered either prior to or during surgery or an invasive procedure. Furthermore, the data presented herein show that bentracimab acts safely for reversal of ticagrelor-enhanced bleeding, even in fragile and elderly patient populations that intrinsically have high incidence rates of adverse effects.

In some aspects, provided herein are methods of reversing ticagrelor-enhanced bleeding in a patient comprising administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient. In some embodiments, the antibody or antigen-binding fragment thereof is administered to the patient during surgery, in a perioperative setting, or during an invasive procedure. In some embodiments, the patient is elderly, has underlying cardiac or pulmonary or metabolic disease, and/or has limited cardiac reserve. In some embodiments, the patient has a minimum percent inhibition of P2Y₁₂ reaction units (PRU) over 4 hours after starting administration of the antibody or antigen-binding fragment thereof that is less than the percent inhibition of PRU before administration. In some aspects, provided herein are methods of reversing ticagrelor-enhanced bleeding in a patient comprising: i) administering to a patient an antibody or antigen-binding fragment thereof that binds to ticagrelor and ii) improving percent inhibition of PRU in the patient. In some embodiments, administration of the antibody or antigen-binding fragment thereof to a population of patients results in a treatment-emergent adverse event related to the administration of the antibody or antigen-binding fragment thereof in less than 3% of patients within the population of patients.

### I. Definitions

The term “amino acid” denotes the group of carboxy α-amino acids, either occurring naturally, i.e., which directly or in form of a precursor can be encoded by a nucleic acid, or occurring non-naturally. The individual naturally occurring amino acids are encoded by nucleic acids consisting of three nucleotides, so called codons or base-triplets. Each amino acid is encoded by at least one codon. This is known as “degeneration of the genetic code”. The term “amino acid” as used within this application denotes the naturally occurring carboxy α-amino acids comprising alanine (three letter code: ala, one letter code: A), arginine (Arg, R), asparagine (Asn, N), aspartic acid (Asp, D), cysteine (Cys, C), glutamine (Gln, Q), glutamic acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). Examples of non-naturally occurring amino acids include, but are not limited to, Aad (alpha-aminoadipic acid), Abu (aminobutyric acid), Ach (alpha-aminocyclohexane-carboxylic acid), Acp (alpha-aminocyclopentane-carboxylic acid), Acpc (1-Aminocyclopropane-1-carboxylic acid), Aib (alpha-aminoisobutyric acid), Aic (2-Aminoindane-2-carboxylic acid; also called 2-2-Aic), 1-1-Aic (1-aminoindane-1-carboxylic acid), (2-aminoindane-2-carboxylic acid), allylglycine (allylGly), alloisoleucine (allo-Ile), Asu (alpha-aminosuberic acid, 2-aminooctanedioc acid), Bip (4-phenyl-phenylalanine-carboxylic acid), BnHP ((2S,4R)-4-hydroxyproline), Cha (beta-cyclohexylalanine), Cit (citrulline), cyclohexylglycine (Chg), cyclopentylalanine, beta-cyclopropyl alanine, Dab (1,4-Diaminobutyric acid), Dap (1,3-Diaminopropionic acid), p (3,3-diphenylalanine-carboxylic acid), 3,3-Diphenylalanine, Di-n-propylglycine (Dpg), 2-Furylalanine, Homocyclohexylalanine (HoCha), Homocitrulline (HoCit), Homocycloleucine, Homoleucin (HoLeu), Homoarginine (HoArg), Homoserine (HoSer), Hydroxyproline, Lys(Ac), (1) Nal (1-Naphtyl Alanine), (2) Nal (2-Naphtyl Alanine), 4-MeO-Apc (1-amino-4-(4-methoxyphenyl)-cyclohexane-1-carboxylic acid), Nor-leucine (Nle), Nva (Norvaline), Omathine, 3-Pal (alpha-amino-3-pyridylalanine-carboxylic acid), 4-Pal (alpha-amino-4-pyridylalanine-carboxylic acid), 3,4,5,F3-Phe (3,4,5-Trifluoro-phenylalanine), 2,3,4,5,6,F5-Phe (2,3,4,5,6-Pentafluoro-phenylalanine), Pqa (4-oxo-6-(1-piperazinyl)-3(4H)-quinazoline-acetic acid (CAS 889958-08-1)), Pyridylalanine, Quinolylalanine, Sarcosine (Sar), Thiazolylalanine, Thienylalanine, Tic (alpha-amino-1,2,3,4,tetrahydroisoquinoline-3-carboxylic acid), Tic(OH), Tle (tertbutylGlycine), and Tyr(Me).

The term “amino acid sequence variant” refers to polypeptides having amino acid sequences that differ to some extent from a native sequence polypeptide. Ordinarily, amino acid sequence variants will possess at least about 70% sequence identity with the native sequence polypeptide. In one embodiment the variant has about 80% or more sequence identity with the native sequence polypeptide. In one embodiment the variant has about 90% or more sequence identity with the native sequence polypeptide. In one embodiment the variant has about 95% or more sequence identity with the native sequence polypeptide. In one embodiment the variant has about 98% or more sequence identity with the native sequence polypeptide. The amino acid sequence variants possess substitutions, deletions, and/or insertions at certain positions within the amino acid sequence of the native amino acid sequence. Amino acids are designated by the conventional names, one-letter and three-letter codes.

The term “antibody fragment” denotes a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The terms “binding site” or “antigen-binding site” as used herein denotes the region(s) of an antibody molecule to which a ligand (e.g. the antigen or antigen fragment of it) actually binds and which is derived from an antibody. The antigen-binding site includes antibody heavy chain variable domains (VH) and/or an antibody light chain variable domain (VL), or pairs of VH/VL.

An antigen-binding site of an antibody of the invention can contain six complementarity determining regions (CDRs) which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences. Also included within the scope of the invention are functional antigen binding sites comprised of fewer CDRs (i.e., where binding specificity is determined by three, four or five CDRs). For example, less than a complete set of 6 CDRs may be sufficient for binding. In some cases, a VH or a VL domain will be sufficient.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, ι, ε, γ, and μ, respectively.

The term “effector functions” denotes those biological activities attributable to the Fc-region of an antibody, which vary with the antibody class. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “effective amount” of an agent, e.g., a pharmaceutical formulation, denotes an amount effective, at dosages and for periods of time necessary, to achieve the desired therapeutic or prophylactic result.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen binding site on the surface of the VH-VL dimer. Collectively, the six hypervariable regions confer antigen-binding specificity to the antibody.

The term “framework”, short “FR”, denotes heavy and light chain variable domain amino acid residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The term “full length antibody” denotes an antibody having a structure substantially similar to a native antibody structure or having heavy chains that contain an Fc-region as defined herein. Native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “patient” as used herein is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., 2007.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “monospecific antibody” denotes an antibody that has one or more binding sites each of which has the same binding specificity, i.e. binds to the same antigen or motif amino acid sequence.

The term “percent inhibition of PRU” or “% inhibition of PRU” is calculated as 100×[(180−PRU)/180]. PRU may refer to PRU that is measured post-treatment with the bentracimab (PRUₜᵣₜ) or the baseline PRU value measured prior to treatment. Percent inhibition of PRU wherein the PRU value measured posttreatment with bentracimab is determined by calculating 100×(180−[PRUₜᵣₜ])/180.

The term “minimum % inhibition of PRU” refers to the minimum value of percent inhibition of PRU over a specified period of time, e.g., 4 hours after starting administration of bentracimab.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

A “polypeptide” is a polymer consisting of amino acids joined by peptide bonds, whether produced naturally or synthetically. Polypeptides of less than about 20 amino acid residues may be referred to as “peptides”, whereas molecules consisting of two or more polypeptides or comprising one polypeptide of more than 100 amino acid residues may be referred to as “proteins”. A polypeptide may also comprise non-amino acid components, such as, but not limited to, carbohydrate groups, metal ions, phosphate groups, or carboxylic acid esters. The non-amino acid components may be added by the cell, in which the polypeptide is expressed, and may vary with the type of cell. Polypeptides are defined herein in terms of their amino acid backbone structure or the nucleic acid encoding the same. Additions such as carbohydrate groups are generally not specified, but may be present nonetheless.

All polypeptide sequences are written according to the generally accepted convention whereby the alpha-N-terminal amino acid residue is on the left and the alpha-C-terminal amino acid residue is on the right. As used herein, the term “N-terminus” refers to the free alpha-amino group of an amino acid in a polypeptide, and the term “C-terminus” refers to the free a-carboxylic acid terminus of an amino acid in a polypeptide. A polypeptide which is N-terminated with a group refers to a polypeptide bearing a group on the alpha-amino nitrogen of the N-terminal amino acid residue. An amino acid which is N-terminated with a group refers to an amino acid bearing a group on the alpha-amino nitrogen.

The term “vector” denotes a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### II. Methods of Reversing Ticagrelor-Enhanced Bleeding Antibodies or Antigen-Binding Fragments Thereof

In some aspects, provided herein are methods of reversing ticagrelor-enhanced bleeding in a patient comprising administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient during surgery, in a perioperative setting, or during an invasive procedure, wherein the antibody or antigen-binding fragment thereof comprises a heavy chain complementarity determining region (CDRH)1, a CDRH2, and a CDRH3 of a variable heavy chain region (VH) comprising the amino acid sequence set forth in SEQ ID NO:1 and a light chain complementarity determining region (CDRL)1, CDRL2, and a CDRL3 of a variable light chain region (VL) comprising the amino acid sequence set forth in SEQ ID NO:02. In some embodiments, the antibody or antigen-binding fragment thereof comprises CDR combinations comprising SEQ ID NO:03 (CDRH1), SEQ ID NO:04 (CDRH2), SEQ ID NO:05 (CDRH3), SEQ ID NO:06 (CDRL1), SEQ ID NO:07 (CDRL2), and SEQ ID NO:08 (CDRL3). In some embodiments, the VH has an amino acid sequence according to SEQ ID NO:01 and the VL has an amino acid sequence according to SEQ ID NO:02. In some embodiments, the antibody or antigen-binding fragment is an antibody. In some embodiments, the antibody or antigen-binding fragment is an antigen-binding fragment. In some embodiments, the antigen-binding fragment is a Fab. In some embodiments, the Fab is bentracimab. In some embodiments, the antibody or antigen-binding fragment thereof is administered to the patient during surgery, in a perioperative setting, or during an invasive procedure. In some embodiments, the antibody or antigen-binding fragment thereof is administered to the patient, wherein the patient is elderly, has underlying cardiac or pulmonary or metabolic disease, and/or has limited cardiac reserve. In some embodiments, the patient has a minimum percent inhibition of P2Y₁₂ reaction units (PRU) over 4 hours after starting administration of the antibody or antigen-binding fragment thereof that is less than the percent inhibition of PRU before administration. In some embodiments, the method comprises i) administering an antibody or antigen-binding fragment thereof that binds to ticagrelor to the patient and ii) improve percent inhibition of PRU in the patient. In some embodiments, the antibody or antigen-binding fragment thereof is administered in the following schedule: 6 g over 10 minutes, followed by 6 g over 4 hours, followed by 6 g over 12 hours. In some embodiments, the method comprises administering 6 g of the antibody or antigen-binding fragment thereof over 10 minutes. In some embodiments, the method comprises a segment comprising administering 6 g of the antibody or antigen-binding fragment thereof over 4 hours. In some embodiments, the method comprises a segment comprising administering 6 g of the antibody or antigen-binding fragment thereof over 12 hours. In some embodiments, the method further comprises an additional segment comprising administering 6 g of the antibody or antigen-binding fragment thereof over 12 hours. In some embodiments, the antibody or antigen-binding fragment thereof is administered in the following schedule: 6 g over 10 minutes, followed by 6 g over 4 hours, followed by 6 g over 12 hours, followed by 6 g over 12 hours. In some embodiments, the additional segment of 6 g of the antibody or antigen-binding fragment over 12 hours is repeated as necessary to reverse ticagrelor-enhanced bleeding. In some embodiments, the additional segment of 6 g of the antibody or antigen-binding fragment over 12 hours is repeated one time, e.g., the antibody or antigen-binding fragment thereof is administered in the following schedule: 6 g over 10 minutes, followed by 6 g over 4 hours, followed by 6 g over 12 hours, followed by 6 g over 12 hours. In some embodiments, the additional administration of 6 g of the antibody or antigen-binding fragment over 12 hours is repeated two times, e.g., the antibody or antigen-binding fragment thereof is administered in the following schedule: 6 g over 10 minutes, followed by 6 g over 4 hours, followed by 6 g over 12 hours, followed by 6 g over 12 hours, followed by 6 g over 12 hours. In some embodiments, the additional segment of 6 g of the antibody or antigen-binding fragment over 12 hours is repeated three or more times. In some embodiments, the antibody or antigen-binding fragment is administered intravenously.

In some aspects, provided herein are methods of reversing ticagrelor-enhanced bleeding in a patient comprising: performing surgery or an invasive procedure on the patient; and administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient during the surgery or during the invasive procedure, wherein the antibody or antigen-binding fragment thereof comprises a heavy chain complementarity determining region (CDRH)1, a CDRH2, and a CDRH3 of a variable heavy chain region (VH) comprising the amino acid sequence set forth in SEQ ID NO:1 and a light chain complementarity determining region (CDRL)1, CDRL2, and a CDRL3 of a variable light chain region (VL) comprising the amino acid sequence set forth in SEQ ID NO:02. In some embodiments, the antibody or antigen-binding fragment thereof comprises CDR combinations comprising SEQ ID NO:03 (CDRH1), SEQ ID NO:04 (CDRH2), SEQ ID NO:05 (CDRH3), SEQ ID NO:06 (CDRL1), SEQ ID NO:07 (CDRL2), and SEQ ID NO:08 (CDRL3). In some embodiments, the VH has an amino acid sequence according to SEQ ID NO:01 and the VL has an amino acid sequence according to SEQ ID NO:02. In some embodiments, the antibody or antigen-binding fragment is an antibody. In some embodiments, the antibody or antigen-binding fragment is an antigen-binding fragment. In some embodiments, the antigen-binding fragment is a Fab. In some embodiments, the Fab is bentracimab. In some embodiments, the patient is in need of the surgery or the invasive produced. In some embodiments, the surgery is a non-deferrable surgery. In some embodiments, the antibody or antigen-binding fragment thereof is administered to the patient, wherein the patient is elderly, has underlying cardiac or pulmonary or metabolic disease, and/or has limited cardiac reserve. In some embodiments, the patient has a minimum percent inhibition of P2Y₁₂ reaction units (PRU) over 4 hours after starting administration of the antibody or antigen-binding fragment thereof that is less than the percent inhibition of PRU before administration. In some embodiments, the method comprises i) administering an antibody or antigen-binding fragment thereof that binds to ticagrelor to the patient and ii) improve percent inhibition of PRU in the patient.

TABLE 1
Summary of Bentracimab Sequences

| Sequence Name | Sequence |
|---|---|
| SEQ ID NO: 01 | QVQLQESGAEVKKPGSSVRVSCKASGGTFDSYS |
| Bentracimab VH | IHWVRQAPGQGLEWMGGIIPAFGTLSSAQDFQA |
| | RVTISADKSTSTAYMELSGLRSEDTAVYYCARG |
| | SFDYYFWSASHPPNDALAIWGQGTLVTVSS |
| SEQ ID NO: 02 | QSVVTQPPSVSAAPGQKVTISCSGSNSDIGNNY |
| Bentracimab VL | VSWYQQLPGTAPKLLIYDNNKRPSGIPDRFSGS |
| | KSGTSATLAITLQAGDEADYYCGTWLYDRAVGL |
| | FGGGTKVTVL |
| SEQ ID NO: 03 | SYSIH |
| Bentracimab | |
| CDRH1 | |
| SEQ ID NO: 04 | GIIPAFGTLSSAQDFQA |
| Bentracimab | |
| CDRH2 | |
| SEQ ID NO: 05 | GSFDYYFWSASHPPNDALAI |
| Bentracimab | |
| CDRH3 | |
| SEQ ID NO: 06 | SGSNSDIGNNYVS |
| Bentracimab | |
| CDRL1 | |
| SEQ ID NO: 07 | DNNKRPS |
| Bentracimab | |
| CDRL2 | |
| SEQ ID NO: 08 | GTWLYDRAVGL |
| Bentracimab | |
| CDRL3 | |

### Dosing and Methods of Administration

In some embodiments, provided herein are methods comprising administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient during surgery, in a perioperative setting, or during an invasive procedure. In some embodiments, the antibody or antigen-binding fragment thereof is administered during surgery. In some embodiments, the surgery has an adverse procedural outcome if hemostasis is impaired. In some embodiments, the surgery is associated with a risk of significant bleeding. In some embodiments, the surgery is cardiac surgery, gastrointestinal surgery, vascular surgery, thoracic surgery, neurosurgery, gastrointestinal surgery, urogenital surgery, or major orthopedic surgery. In some embodiments the surgery is coronary artery bypass graft (CABG), cardiac surgery, gastrointestinal surgery, coronary artery procedure, repair of aortic dissection, and/or cardiac valve replacement. In some embodiments, the surgery is CABG. In some embodiments, the surgery is mediastinal surgery. In some embodiments, the surgery or invasive procedure is invasive cardiothoracic procedures, vascular surgery, abdominal surgery, and/or surgery for traumatic injuries. In some embodiments, the antibody or antigen-binding fragment thereof is administered to the patient, wherein the patient is elderly, has underlying cardiac or pulmonary or metabolic disease, and/or has limited cardiac reserve. In some embodiments, the patient has a minimum percent inhibition of P2Y₁₂ reaction units (PRU) over 4 hours after starting administration of the antibody or antigen-binding fragment thereof that is less than the percent inhibition of PRU before administration. In some embodiments, administration of the antibody or antigen-binding fragment thereof results in improved percent inhibition of PRU in the patient. In some embodiments, the antibody or antigen-binding fragment thereof is administered in the following schedule: 6 g over 10 minutes, followed by 6 g over 4 hours, followed by 6 g over 12 hours. In some embodiments, a total of 18 grams of the antibody or antigen-binding fragment thereof is administered to the patient. In some embodiments, the antibody or antigen-binding fragment thereof is administered over 16 hours and 10 minutes. In some embodiments, the method comprises a segment administering 6 g of the antibody or antigen-binding fragment thereof over 10 minutes. In some embodiments, the method comprises a segment comprising administering 6 g of the antibody or antigen-binding fragment thereof over 4 hours. In some embodiments, the method comprises a segment comprising administering 6 g of the antibody or antigen-binding fragment thereof over 12 hours. In some embodiments, the method further comprises an additional segment comprising administering 6 g of the antibody or antigen-binding fragment thereof over 12 hours. In some embodiments, the antibody or antigen-binding fragment thereof is administered in the following schedule: 6 g over 10 minutes, followed by 6 g over 4 hours, followed by 6 g over 12 hours, followed by 6 g over 12 hours. In some embodiments, the additional segment of 6 g of the antibody or antigen-binding fragment over 12 hours is repeated one, or two, or three or more times. In some embodiments, the additional segment of 6 g of the antibody or antigen-binding fragment over 12 hours is repeated one time, e.g., the antibody or antigen-binding fragment thereof is administered in the following schedule: 6 g over 10 minutes, followed by 6 g over 4 hours, followed by 6 g over 12 hours, followed by 6 g over 12 hours. In some embodiments, a total of 24 grams of the antibody or antigen-binding fragment thereof is administered to the patient. In some embodiments, the antibody or antigen-binding fragment thereof is administered over 28 hours and 10 minutes. In some embodiments, the additional administration of 6 g of the antibody or antigen-binding fragment over 12 hours is repeated two times, e.g., the antibody or antigen-binding fragment thereof is administered in the following schedule: 6 g over 10 minutes, followed by 6 g over 4 hours, followed by 6 g over 12 hours, followed by 6 g over 12 hours, followed by 6 g over 12 hours. In some embodiments, a total of 36 grams of the antibody or antigen-binding fragment thereof is administered to the patient. In some embodiments, the antibody or antigen-binding fragment thereof is administered over 30 hours and 10 minutes. In some embodiments, the additional segment of 6 g of the antibody or antigen-binding fragment over 12 hours is repeated three or more times. In some embodiments, the additional segment is administered in a patient in need thereof based on signs or symptoms of ongoing hemorrhage or risk of rebleeding. In some embodiments, the antibody or antigen-binding fragment is administered intravenously.

In some embodiments, the methods comprise performing surgery or an invasive procedure on the patient, wherein the antibody or antigen binding fragment thereof is administered during surgery.

In some embodiments, provided herein are methods comprising performing a surgery or invasive procedure on a patient and, administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient during surgery or during an invasive procedure. In some embodiments, the surgery has an adverse procedural outcome if hemostasis is impaired. In some embodiments, the surgery is associated with a risk of significant bleeding. In some embodiments, the surgery is cardiac surgery, gastrointestinal surgery, vascular surgery, thoracic surgery, neurosurgery, gastrointestinal surgery, urogenital surgery, or major orthopedic surgery. In some embodiments the surgery is coronary artery bypass graft (CABG), cardiac surgery, gastrointestinal surgery, coronary artery procedure, repair of aortic dissection, and/or cardiac valve replacement. In some embodiments, the surgery is CABG. In some embodiments, the surgery is mediastinal surgery. In some embodiments, the surgery or invasive procedure is invasive cardiothoracic procedures, vascular surgery, abdominal surgery, and/or surgery for traumatic injuries. In some embodiments, the antibody or antigen-binding fragment thereof is administered to the patient, wherein the patient is elderly, has underlying cardiac or pulmonary or metabolic disease, and/or has limited cardiac reserve. In some embodiments, the patient has a minimum percent inhibition of P2Y₁₂ reaction units (PRU) over 4 hours after starting administration of the antibody or antigen-binding fragment thereof that is less than the percent inhibition of PRU before administration. In some embodiments, administration of the antibody or antigen-binding fragment thereof results in improved percent inhibition of PRU in the patient. In some embodiments, the antibody or antigen binding fragment thereof is administered at a concentration of about 100 mg/mL.

In some embodiments, the antibody or antigen-binding fragment thereof is administered in the following schedule: 12 g infused over 10 minutes, followed by 12 g over 6 hours, followed by 12 g over 18 hours, wherein the patient has been treated with a moderate or strong CYP3A inhibitor and is administered the antibody or antigen-binding fragment thereof within 5 half-lives of the most recent CYP3A dose. In some embodiments, the strength of the CYP3A inhibitor is classified based on its effect on the in vivo fold-change in the plasma area under the curve (AUC) of a sensitive index substrate of CYP3A. An inhibitor of CYP3A4 is classified as a weak inhibitor if the drug increases the AUC of sensitive index substrates of CYP3A by ≥1.25- to <2-fold. An inhibitor of CYP3A4 is classified as a moderate inhibitor if the drug increases the AUC of sensitive index substrates of CYP3A by ≥2- to <5-fold. An inhibitor of CYP3A4 is classified as a strong inhibitor if the drug increases the AUC of sensitive index substrates of CYP3A≥5-fold. In some embodiments, the moderate or strong CYP3A inhibitor is selected from the group consisting of: adagrasib, atazanavir, ceritinib, clarithromycin, cobicistat and cobicistat-containing coformulations, darunavir, idelalisib, indinavir, itraconazole, ketoconazole, levoketoconazole, lonafarnib, lopinavir, mifepristone, nefazodone, nelfinavir, nirmatrelvir-ritonavir, ombitasvir-paritaprevir-ritonavir, ombitasvir-paritaprevir-ritonavir plus dasabuvir, Posaconazole. Ritonavir and ritonavir-containing coformulations, saquinavir, tucatinib, voriconazole, and amiodarone, aprepitant, berotralstat, cimetidine, conivaptan, crizotinib, cyclosporine, diltiazem, duvelisib, dronedarone, erythromycin, fedratinib. Fluconazole, fosamprenavir, fosaprepitant, fosnetupitant-palonosetron. Grapefruit juice, imatinib, isavuconazole (isavuconazonium sulfate), Lefamulin, letermovir, netupitant, nilotinib, nirogecestat, ribociclib, schisandra, and Verapamil. In some embodiments, the strong CYP3A inhibitor is selected from the group consisting of: adagrasib, atazanavir, ceritinib, clarithromycin, cobicistat and cobicistat-containing coformulations, darunavir, idelalisib, indinavir, itraconazole, ketoconazole, levoketoconazole, lonafarnib, lopinavir, nefazodone, nelfinavir, nirmatrelvir-ritonavir, ombitasvir-paritaprevir-ritonavir, ombitasvir-paritaprevir-ritonavir plus dasabuvir, Posaconazole. Ritonavir and ritonavir-containing coformulations, saquinavir, tucatinib, and voriconazole. In some embodiments, the strong CYP3A inhibitor is mifepristone when mifepristone is chronically. In some the moderate CYP3A inhibitor is selected from the group consisting of: amiodarone, aprepitant, berotralstat, cimetidine, conivaptan, crizotinib, cyclosporine, diltiazem, duvelisib, dronedarone, erythromycin, fedratinib. Fluconazole, fosamprenavir, fosaprepitant, fosnetupitant-palonosetron. Grapefruit juice, imatinib, isavuconazole (isavuconazonium sulfate), Lefamulin, letermovir, netupitant, nilotinib, nirogecestat, ribociclib, schisandra, and Verapamil. In some embodiments, the CYP3A inhibitor is diltiazem or verapamil. In some embodiments, a total of 36 grams of the antibody or antigen-binding fragment thereof is administered to the patient. In some embodiments, the antibody or antigen-binding fragment thereof is administered over 24 hours and 10 minutes. In some embodiments, the method comprises a segment administering 12 g of the antibody or antigen-binding fragment thereof over 10 minutes. In some embodiments, the method comprises a segment comprising administering 12 g of the antibody or antigen-binding fragment thereof over 6 hours. In some embodiments, the method comprises a segment comprising administering 12 g of the antibody or antigen-binding fragment thereof over 18 hours. In some embodiments, the method further comprises an additional segment comprising administering 12 g of the antibody or antigen-binding fragment thereof over 18 hours. In some embodiments, the antibody or antigen-binding fragment thereof is administered in the following schedule: 12 g infused over 10 minutes, followed by 12 g over 6 hours, followed by 12 g over 18 hours. In some embodiments, the additional segment of 12 g of the antibody or antigen-binding fragment over 18 hours is repeated one or two, or three or more times. In some embodiments, the additional segment of 12 g of the antibody or antigen-binding fragment over 18 hours is repeated one time, e.g., the antibody or antigen-binding fragment thereof is administered in the following schedule: 12 g infused over 10 minutes, followed by 12 g over 6 hours, followed by 12 g over 18 hours, followed by 12 g over 18 hours. In some embodiments, a total of 48 grams of the antibody or antigen-binding fragment thereof is administered to the patient. In some embodiments, the antibody or antigen-binding fragment thereof is administered over 42 hours and 10 minutes. In some embodiments, the additional administration of 12 g of the antibody or antigen-binding fragment over 18 hours is repeated two times, e.g., the antibody or antigen-binding fragment thereof is administered in the following schedule: 12 g infused over 10 minutes, followed by 12 g over 6 hours, followed by 12 g over 18 hours, followed by 12 g over 18 hours, followed by 12 g over 18 hours. In some embodiments, a total of 60 grams of the antibody or antigen-binding fragment thereof is administered to the patient. In some embodiments, the antibody or antigen-binding fragment thereof is administered over 60 hours and 10 minutes. In some embodiments, the additional segment of 12 g of the antibody or antigen-binding fragment over 18 hours is repeated three or more times. In some embodiments, the additional segment is administered in a patient in need thereof based on signs or symptoms of ongoing hemorrhage or risk of rebleeding. In some embodiments, the antibody or antigen-binding fragment is administered intravenously.

In some embodiments, the antibody or antigen-binding fragment thereof that binds to ticagrelor or a metabolite or derivative thereof is administered in a perioperative setting. In some embodiments, a perioperative setting refers to a setting during a perioperative period, which is the time surrounding a surgical act. In some embodiments, the perioperative period comprises the period between when the patient goes to the hospital or clinic and when the patient returns home from surgery, e.g., the period ward admission, anesthesia, surgery, recovery. In some embodiments, the perioperative setting is a preoperative (before surgery), intraoperative (during surgery), or postoperative (after surgery) setting. In some embodiments, the antibody or antigen-binding fragment thereof is administered between about 0 to 36 hours before surgery. In some embodiments, the antibody or antigen-binding fragment thereof is administered between about 0 to 10 minutes before surgery. In some embodiments, the antibody or antigen-binding fragment thereof is administered between about 0 to about 10 minutes before surgery. In some embodiments, the antibody or antigen-binding fragment thereof is administered between about 0 to about 1 hour before surgery. In some embodiments, the antibody or antigen-binding fragment thereof is administered between about 1 to about 4 hours before surgery. In some embodiments, the antibody or antigen-binding fragment thereof is administered between about 4 to about 8 hours before surgery. In some embodiments, the antibody or antigen-binding fragment thereof is administered between about 8 to about 12 hours before surgery. In some embodiments, the antibody or antigen-binding fragment thereof is administered between about 12 to about 16 hours before surgery. In some embodiments, the antibody or antigen-binding fragment thereof is administered between about 16 to about 20 hours before surgery. In some embodiments, the antibody or antigen-binding fragment thereof is administered between about 20 to about 24 hours before surgery. In some embodiments, the antibody or antigen-binding fragment thereof is administered between about 24 to about 30 hours before surgery. In some embodiments, the antibody or antigen-binding fragment thereof is administered between about 30 to about 36 hours before surgery. In some embodiments, the antibody or antigen-binding fragment thereof is administered to the patient, wherein the patient is elderly, has underlying cardiac or pulmonary or metabolic disease, and/or has limited cardiac reserve. In some embodiments, administration of the antibody or antigen-binding fragment thereof results in improved percent inhibition of PRU in the patient. In some embodiments, the antibody or antigen-binding fragment thereof is administered in the following schedule: 6 g infused over 10 minutes, followed by 6 g over 4 hours, followed by 6 g over 12 hours.

In some embodiments, the patient in need of reversal of ticagrelor-enhanced bleeding does not receive treatment with the antibody or antigen-binding fragment thereof prior to surgery. In some embodiments, the patient in need of reversal of ticagrelor-enhanced bleeding does not receive treatment with the antibody or antigen-binding fragment thereof prior to the start of surgery. In some embodiments, the antibody or antigen-binding fragment thereof is administered between about 0 to 36 hours after surgery. In some embodiments, the antibody or antigen-binding fragment thereof is administered between about 0 to 10 minutes after surgery. In some embodiments, the antibody or antigen-binding fragment thereof is administered between about 0 to about 10 minutes after surgery. In some embodiments, the antibody or antigen-binding fragment thereof is administered between about 0 to about 1 hour after surgery. In some embodiments, the antibody or antigen-binding fragment thereof is administered between about 1 to about 4 hours after surgery. In some embodiments, the antibody or antigen-binding fragment thereof is administered between about 4 to about 8 hours after surgery. In some embodiments, the antibody or antigen-binding fragment thereof is administered between about 8 to about 12 hours after surgery. In some embodiments, the antibody or antigen-binding fragment thereof is administered between about 12 to about 16 hours after surgery. In some embodiments, the antibody or antigen-binding fragment thereof is administered between about 16 to about 20 hours after surgery. In some embodiments, the antibody or antigen-binding fragment thereof is administered between about 20 to about 24 hours after surgery. In some embodiments, the antibody or antigen-binding fragment thereof is administered between about 24 to about 30 hours after surgery. In some embodiments, the antibody or antigen-binding fragment thereof is administered between about 30 to about 36 hours after surgery.

In some embodiments, the antibody or antigen-binding fragment thereof that binds to ticagrelor or a metabolite or derivative thereof is administered during an invasive procedure. In some embodiments, invasive procedure is a procedure that enters the body by cutting or puncturing skin or by inserting instruments into the body. In some embodiments, the invasive procedure is percutaneous cardiac intervention or another cardiac or vascular procedure. In some embodiments, the invasive procedure is a cardiac valve replacement. In some embodiments, the antibody or antigen-binding fragment thereof is administered to the patient, wherein the patient is elderly, has underlying cardiac or pulmonary or metabolic disease, and/or has limited cardiac reserve. In some embodiments, administration of the antibody or antigen-binding fragment thereof results in improved percent inhibition of PRU in the patient. In some embodiments, the antibody or antigen-binding fragment thereof is administered in the following schedule: 6 g infused over 10 minutes, followed by 6 g over 4 hours, followed by 6 g over 12 hours.

In some embodiments, the antibody or antigen-binding fragment thereof that binds to ticagrelor or a metabolite or derivative thereof is administered to the patient intravenously. In some embodiments, the antibody or antigen-binding fragment thereof is administered via a standard IV bag and IV pump, or by syringe using a syringe pump. In some embodiments, the antibody or antigen-binding fragment thereof is administered intravenously over about 15 minutes to about 36 hours. In some embodiments, the antibody or antigen-binding fragment thereof is administered in two or more segments. In some embodiments, the antibody or antigen-binding fragment thereof is administered in three segments. In some embodiments, the first segment is a bolus. In some embodiments, the bolus is administered as 6 g infused over 10 minutes. In some embodiments, the administration rates for each of the segments differ. In some embodiments, the administration rates for each of the segments differ for successive segments of the infusion. In some embodiments, the pharmaceutical composition is administered in three or more segments, wherein the administration rates for each of the segments differ for successive segments of the infusion.

In some embodiments, the antibody or antigen-binding fragment thereof that binds to ticagrelor or a metabolite or derivative thereof is administered in the following schedule: 6 g infused over 10 minutes, followed by 6 g over 4 hours, followed by 6 g over 12 hours. In some embodiments, the antibody or antigen-binding fragment thereof is administered to the patient during surgery, in a perioperative setting, or during an invasive procedure. In some embodiments, the antibody or antigen-binding fragment thereof is administered to the patient, wherein the patient is elderly, has underlying cardiac or pulmonary or metabolic disease, and/or has limited cardiac reserve. In some embodiments, the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, administration of the antibody or antigen-binding fragment thereof results in improved percent inhibition of PRU in the patient.

In some embodiments, e.g., if extended reversal is needed based on signs or symptoms of ongoing hemorrhage or risk of re-bleeding, the maintenance infusion of bentracimab is extended. In some embodiments, infusion of bentracimab is extended by 6 g over 12 hours. In some embodiments, infusion of bentracimab is extended by 12 g over 18 hours. In some embodiments, the antibody or antigen-binding fragment thereof is administered in the following schedule: 6 g infused over 10 minutes, followed by 6 g over 4 hours, followed by 6 g over 12 hours, followed by 6 g over 12 hours. In some embodiments, the antibody or antigen-binding fragment thereof is administered in the following schedule: 12 g infused over 10 minutes, followed by 12 g over 6 hours, followed by 12 g over 18 hours, followed by 12 g over 18 hours, wherein the patient has been treated with a moderate or strong CYP3A inhibitor and is administered the antibody or antigen-binding fragment thereof within 5 half-lives of the most recent CYP3A dose.

In some embodiments, the patient has been treated with a moderate or strong CYP3A inhibitor, and the antibody or antigen-binding fragment thereof that binds to ticagrelor or a metabolite or derivative thereof is administered in the following schedule: 12 g infused over 10 minutes, followed by 12 g over 6 hours, followed by 12 g over 18 hours. In some embodiments, the patient is administered the antibody or antigen-binding fragment thereof within 5 half-lives of the most recent CYP3A dose. In some embodiments, the CYP3A inhibitor is diltiazem or verapamil.

In some embodiments, the antibody or antigen-binding fragment thereof that binds to ticagrelor or a metabolite or derivative thereof is administered as a high-concentration liquid. In some embodiments, the antibody or antigen-binding fragment thereof is administered at a concentration of 100 mg/mL within a formulation comprising 25 mM L-histidine/L-histidine HCl monohydrate, 290 mM sucrose, 0.05% polysorbate 80, pH 6.0. In some embodiments, the method comprises administering a total 18 g intravenous infusion of the antibody or antigen-binding fragment thereof that binds to ticagrelor comprising an initial IV bolus of 6 g (60 mL) infused over 10 minutes for rapid reversal. In some embodiments, the method further comprises administering a 6 g (60 mL) loading dose over 4 hours immediately following the bolus infusion, followed by a maintenance dose of 6 g (60 ml) infused over the next 12 hours immediately following completion of the loading dose. In some embodiments, a total dose of 18 g (180 mL) is administered over an infusion time of 16 hours and 10 minutes.

In some embodiments, the method of reversing ticagrelor-enhanced bleeding comprising administering the antibody or antigen-binding fragment thereof that binds to ticagrelor or a metabolite or derivative thereof further comprises administering to the patient acetylsalicylic acid. In some embodiments, the dose of acetylsalicylic acid is 0 mg to 100 mg. In some embodiments, the patient receives chronic treatment with acetylsalicylic acid.

In some embodiments, the antibody or antigen-binding fragment thereof that binds to ticagrelor or a metabolite or derivative thereof is administered to a patient who previously received ticagrelor treatment. In the management of ACS, ticagrelor treatment is initiated with a 180 mg loading dose, followed by 90 mg twice daily (bid) during the first year after an ACS event. After one year, ticagrelor is administered 60 mg twice daily. In patients with coronary artery disease but no prior stroke or myocardial infarction, ticagrelor is administered 60 mg twice daily. In some embodiments, e.g., for patients with ASC or high risk transient ischaemic attack (TIA), 180 mg of ticagrelor is coadministered with 300-325 mg of acetylsalicyclic acid, or aspirin, within 24 hours in an acute setting, and the patient receives 90 mg ticagrelor and 81 mg of acetylsalicyclic acid for up to 30 days.

In some embodiments, the antibody or antigen-binding fragment thereof that binds to ticagrelor or a metabolite or derivative thereof is administered within about three days prior to the most recent dose of ticagrelor. In some embodiments, the antibody or antigen-binding fragment thereof is administered within about two days prior to the most recent dose of ticagrelor. In some embodiments, the antibody or antigen-binding fragment thereof is administered within about one day prior to the last dose of ticagrelor. In some embodiments, the antibody or antigen-binding fragment thereof is administered between about one to about three days prior to the last dose of ticagrelor. In some embodiments, the antibody or antigen-binding fragment thereof is administered between about two to about three days prior to the last dose of ticagrelor. In some embodiments, the antibody or antigen-binding fragment thereof is administered between about one to about two days prior to the last dose of ticagrelor. In some embodiments, the antibody or antigen-binding fragment thereof is administered on the same day as the most recent dose of ticagrelor. In some embodiments, the patient was previously treated with a daily dose of between about 60 mg and about 360 mg of ticagrelor. In some embodiments, the patient was previously treated with a daily dose of between about 60 mg of ticagrelor. In some embodiments, the patient was previously treated with a daily dose of between about 90 mg of ticagrelor. In some embodiments, the patient was previously treated with a daily dose of between about 180 mg of ticagrelor. In some embodiments, the patient was previously treated with a daily dose of between about 60 mg and about 90 mg of ticagrelor. In some embodiments, the patient was previously treated with a daily dose of between about 60 mg and about 180 mg of ticagrelor. In some embodiments, the patient was previously treated with a daily dose of between about 90 mg and about 180 mg of ticagrelor. In some embodiments, the patient was previously treated with a daily dose of between about 180 mg and about 360 mg of ticagrelor.

In some embodiments, the method of reversing ticagrelor-enhanced bleeding in a patient comprising administering an antibody or antigen-binding fragment thereof that binds to ticagrelor or a metabolite or derivative thereof further comprises restarting ticagrelor treatment after administering the antibody or antigen-binding fragment thereof. In some embodiments, treatment with ticagrelor is restarted subsequent to administration of the antibody or antigen-binding fragment thereof that binds to ticagrelor or a metabolite or derivative thereof. In some embodiments, treatment with ticagrelor is restarted 0 to 7 days after administering the antibody or antigen-binding fragment thereof, preferably 8 hours to 3 days after administering the antibody or antigen-binding fragment thereof. In some embodiments, treatment with ticagrelor is restarted 0-10 minutes subsequent to administration of the antibody or antigen-binding fragment thereof which binds to ticagrelor. In some embodiments, treatment with ticagrelor is restarted 10 minutes to 1 hour subsequent to administration of the antibody or antigen-binding fragment thereof which binds to ticagrelor. In some embodiments, treatment with ticagrelor is restarted 1 hours to 3 hours subsequent to administration of the antibody or antigen-binding fragment thereof which binds to ticagrelor. In some embodiments, treatment with ticagrelor is restarted 3 hours to 6 hours subsequent to administration of the antibody or antigen-binding fragment thereof which binds to ticagrelor. In some embodiments, treatment with ticagrelor is restarted 6 hours to 12 hours subsequent to administration of the antibody or antigen-binding fragment thereof which binds to ticagrelor. In some embodiments, treatment with ticagrelor is restarted 12 hours to 24 hours subsequent to administration of the antibody or antigen-binding fragment thereof which binds to ticagrelor. In some embodiments, treatment with ticagrelor is restarted more than 1 day subsequent to administration of the antibody or antigen-binding fragment thereof which binds to ticagrelor. In some embodiments, treatment with ticagrelor is restarted 1 day to 3 days subsequent to administration of the antibody or antigen-binding fragment thereof which binds to ticagrelor. In some embodiments, treatment with ticagrelor is restarted 1 day to 7 days subsequent to administration of the antibody or antigen-binding fragment thereof which binds to ticagrelor. In some embodiments, treatment with ticagrelor is restarted 1 week to 2 weeks subsequent to administration of the antibody or antigen-binding fragment thereof which binds to ticagrelor. In some embodiments, the method further comprising restarting ticagrelor treatment in a patient who has a minimum percent inhibition of P2Y₁₂ reaction units (PRU) over 4 hours after starting administration of the antibody or antigen-binding fragment thereof that is less than the percent inhibition of PRU before administration.

In some embodiments, The recommended total dose of bentracimab is 18 g (180 mL at 100 mg/mL), provided as three separate vials each containing 6 g (60 mL at 100 mg/mL) bentracimab (see Table 51). All three vials may be provided together in one carton. In some embodiments, the vials comprise 6 g each.

TABLE 51
Standard Dose

| | Patient | |
|---|---|---|
| Dose | Population | Dosing |
| 18 g | Patients treated with ticagrelor when reversal | Total dose (18 g, 180 mL at 100 mg/mL) |
| | of the antiplatelet effects of ticagrelor and its | 6 g (60 mL at 100 mg/mL) over 10 minutes |
| | active metabolite is needed: | Followed by 6 g (60 mL at 100 mg/mL) over 4 |
| | In patients requiring nondeferrable | hours |
| | surgery/invasive procedure | Followed by 6 g (60 mL at 100 mg/mL) over 12 |
| | In patients experiencing major bleeding (1) | hours |

In some embodiments, the recommended dose of bentracimab is adjusted in patients receiving a moderate to strong CYP3A inhibitor (see Table 52)

TABLE 52
Adjusted Dosing Regimens

| Dose | Patient Population | Dose Options |
|---|---|---|
| 24 g | Patients who do not achieve | After the end of infusion of the 18 g (180 mL at 100 mg/mL) |
| | effective hemostasis at the | bentracimab dose (Table 51), follow with an additional 6 g |
| | end of bentracimab 18 g (180 | (60 mL at 100 mg/mL) of RETRIG infused over 12 hours, for |
| | mL at 100 mg/mL) infusion | a total dose of 24 g (240 mL at 100 mg/mL) |
| 36 g | Patients on moderate or | Total dose 36 g (360 mL at 100 mg/mL) |
| | strong CYP3A inhibitors | 12 g (120 mL at 100 mg/mL) over 10 minutes |
| | | Followed by 12 g (120 mL at 100 mg/mL) over 6 hours |
| | | Followed by 12 g (120 mL at 100 mg/mL) over 18 hours |

### Articles of Manufacture

Provided herein are articles of manufacture comprising a vial comprising a monoclonal antibody fragment that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof, wherein the monoclonal antibody comprises a variable heavy chain region (VH) comprising the amino acid sequence set forth in SEQ ID NO:01 and a variable light chain region (VL) comprising the amino acid sequence set forth in SEQ ID NO:02, wherein the vial comprises about 6 g of the monoclonal antibody fragment. In some embodiments, the VH has an amino acid sequence according to SEQ ID NO:01 and the VL has an amino acid sequence according to SEQ ID NO:02. In some embodiments, the monoclonal antibody fragment is a Fab. In some embodiments, the Fab is bentracimab.

In some embodiments, the article of manufacture comprises three vials, each vial comprising about 6 g of the monoclonal antibody fragment. In some embodiments, the monoclonal antibody fragment is a Fab. In some embodiments, the Fab is bentracimab.

In some embodiments, the article of manufacture comprises the monoclonal antibody fragment in a pharmaceutical composition. In some embodiments, the monoclonal antibody fragments is in a pharmaceutical composition at a concentration of 100 mg/mL. In some embodiments, the article of manufacture comprises 60 mL of the monoclonal antibody fragment at a concentration of 100 mg/mL. In some embodiments, the monoclonal antibody fragment is a Fab. In some embodiments, the Fab is bentracimab.

In some embodiments, the article of manufacture comprises three vials, each vial comprising about 6 g of the monoclonal antibody fragment. In some embodiments, the article of manufacture comprises the monoclonal antibody fragment in a pharmaceutical composition. In some embodiments, the monoclonal antibody fragments is in a pharmaceutical composition at a concentration of 100 mg/mL. In some embodiments, the article of manufacture comprises three vials, each vial comprising 60 mL of the monoclonal antibody fragment at a concentration of 100 mg/mL. In some embodiments, the three vials.

In some embodiments, the article of manufacture is carton comprising a treatment course of the monoclonal antibody fragment. In some embodiments, the carton comprises a 18 g treatment course comprising three vials of the monoclonal antibody fragment. In some embodiments, each of the three vials comprise 6 g of the monoclonal antibody fragment. In some embodiments, each of the three vials comprise a pharmaceutical composition at a concentration of 100 mg/mL of the monoclonal antibody fragment. In some embodiments, each vial comprises 60 mL of the monoclonal antibody fragment at a concentration of 100 mg/mL. In some embodiments, the monoclonal antibody fragment is a Fab. In some embodiments, the Fab is bentracimab.

In some embodiments, the article of manufacture comprises instructions for treating patients treated with ticagrelor when reversal of the antiplatelet effects of ticagrelor and its active metabolite is needed. In some embodiments, the patient is in need of nondeferrable surgery or an invasive procedure or in experiencing major bleeding. In some embodiments, the article of manufacture comprises instructions for treating patients requiring nondeferrable surgery or invasive procedure or in patients experiencing major bleeding.

In some embodiments, the article of manufacture comprises instructions for treating patients who do not achieve effective platelet homeostasis at the end of a single treatment course. In some embodiments, the article of manufacture comprises instructions for treating patients on moderate or strong CYP3A inhibitors.

### Patient Populations

In some embodiments, the methods provided herein comprise administering an antibody or antigen binding fragment that binds to ticagrelor or a metabolite or derivative thereof to a patient that has, or is at risk of, uncontrolled major or life-threatening bleeding. In some embodiments, the patient is undergoing urgent surgery or invasive procedure. In some embodiments, the method described herein comprises administering an antibody or antigen-binding fragment thereof that binds to ticagrelor to a patient, wherein the patient is elderly, has underlying cardiac or pulmonary or metabolic disease, and/or has limited cardiac reserve. In some embodiments, the methods comprise performing a surgery or an invasive procedure on the patients, wherein the antibody or antigen binding fragment thereof is administered during surgery.

In some embodiments, the methods provided herein comprise performing a surgery or an invasive procedure on a patient; and administering an antibody or antigen binding fragment that binds to ticagrelor or a metabolite or derivative thereof to a patient that has, or is at risk of, uncontrolled major or life-threatening bleeding. In some embodiments, the surgery is an urgent surgery. In some embodiments, the method described herein comprises administering an antibody or antigen-binding fragment thereof that binds to ticagrelor to a patient, wherein the patient is elderly, has underlying cardiac or pulmonary or metabolic disease, and/or has limited cardiac reserve. In some embodiments, the patient has cardiovascular disease. In some embodiments, the patient has concomitant hypertension and/or diabetes.

In some embodiments, the patient receiving the antibody or antigen binding fragment that binds to ticagrelor or a metabolite or derivative thereof has or is at risk of, one or more of shock, acute coronary syndrome, myocardial infarction, suspected myocardial infarction, unstable angina, or stroke. In some embodiments, the patient receiving the antibody or antigen binding fragment that binds to ticagrelor or a metabolite or derivative thereof is undergoing percutaneous coronary interventions (PCI) and/or is at risk of stent thrombosis. In some embodiments, the stroke is hemorrhagic stroke. In some embodiments, the stroke is ischemic stroke. In some embodiments, the patient is undergoing or requires surgery. In some embodiments, the surgery is associated with a risk of significant bleeding. In some embodiments, the surgery is cardiac surgery, gastrointestinal surgery, vascular surgery, thoracic surgery, neurosurgery, gastrointestinal surgery, urogenital surgery, or major orthopedic surgery. In some embodiments the surgery is coronary artery bypass graft (CABG), cardiac surgery, gastrointestinal surgery, coronary artery procedure, repair of aortic dissection, and/or cardiac valve replacement. In some embodiments, the surgery is CABG. In some embodiments, the surgery is mediastinal surgery. In some embodiments, the surgery or invasive procedure is invasive cardiothoracic procedures, vascular surgery, abdominal surgery, and/or surgery for traumatic injuries. In some embodiments, the patient is undergoing or requires PCI or CABG. In some embodiments, the patient who is undergoing or requires PCI is at risk of stent thrombosis. In some embodiments, the antibody or antigen binding fragment that binds to ticagrelor or a metabolite or derivative thereof is administered to reduce risk of stent thrombosis in patients undergoing PCI. In some embodiments, the antibody or antigen binding fragment that binds to ticagrelor or a metabolite or derivative thereof is administered in combination with acetylsalicylic acid (aspirin). In some embodiments, the antibody or antigen binding fragment that binds to ticagrelor or a metabolite or derivative thereof is administered not in combination with acetylsalicylic acid (aspirin). In some embodiments, the patient is at risk of major bleeding associated with any of surgery or invasive procedure described herein. In some embodiments, the antibody or antigen-binding fragment thereof is administered to the patient during surgery, in a perioperative setting, or during an invasive procedure. In some embodiments, administration of the antibody or antigen-binding fragment thereof results in improved percent inhibition of PRU in the patient. In some embodiments, the antibody or antigen-binding fragment thereof is administered in the following schedule: 6 g infused over 10 minutes, followed by 6 g over 4 hours, followed by 6 g over 12 hours. In some embodiments, the antibody or antigen-binding fragment thereof is administered to the patient, wherein the patient is elderly, has underlying cardiac or pulmonary or metabolic disease, and/or has limited cardiac reserve. In some embodiments, the patient has a minimum percent inhibition of PRU over 4 hours after starting administration of the antibody or antigen-binding fragment thereof that is less than the percent inhibition of PRU before administration, thereby reversing ticagrelor-enhanced bleeding. In some embodiments, administration of the antibody or antigen-binding fragment thereof results in improved percent inhibition of PRU in the patient.

Ticagrelor is indicated to reduce the rate of cardiovascular death, myocardial infarction, and stroke in patients with acute coronary syndrome (ACS) or a history of myocardial infarction (MI). In some embodiments, the antibody or antigen-binding fragment thereof is administered to the patient during surgery, in a perioperative setting, or during an invasive procedure. In some embodiments, administration of the antibody or antigen-binding fragment thereof results in improved percent inhibition of PRU in the patient. In some embodiments, the patient has concomitant hypertension and/or diabetes. In some embodiments, the antibody or antigen-binding fragment thereof is administered in the following schedule: 6 g infused over 10 minutes, followed by 6 g over 4 hours, followed by 6 g over 12 hours. In some embodiments, the antibody or antigen-binding fragment thereof is administered to the patient, wherein the patient is elderly, has underlying cardiac or pulmonary or metabolic disease, and/or has limited cardiac reserve. In some embodiments, the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, administration of the antibody or antigen-binding fragment thereof results in improved percent inhibition of PRU in the patient.

In some embodiments, the patient receiving the antibody or antigen binding fragment that binds to ticagrelor or a metabolite or derivative thereof has or is at risk of acute coronary syndrome. The term acute coronary syndrome (ACS) is applied to patients in whom there is a suspicion or confirmation of acute myocardial ischemia or infarction. In some embodiments, ACS is non-ST-elevation myocardial infarction (NSTEMI), ST-elevation MI (STEMI), or unstable angina. The terms STEMI and NSTEMI are used in patients who present with clinical characteristics compatible with myocardial ischemia and who demonstrate elevated troponin levels in the blood. In some embodiments, unstable angina is new onset angina, rest angina, early post-MI angina, postrevascularization angina, periprocedural angina, and/or late angina. In some embodiments, the patient has a history of myocardial infarction. In some embodiments, the myocardial infarction is selected from the group consisting of: Type 1: Spontaneous MI caused by ischemia due to a primary coronary event (e.g., plaque rupture, erosion, or fissuring; coronary dissection), Type 2: Ischemia due to increased oxygen demand (e.g., hypertension), or decreased supply (e.g., coronary artery spasm or embolism, arrhythmia, hypotension), Type 3: Related to sudden unexpected cardiac death, Type 4a: Associated with percutaneous coronary intervention (signs and symptoms of myocardial infarction with cTn values >5×99^{th} percentile upper reference limit [URL]), Type 4b: Associated with documented stent thrombosis, Type 5: Associated with coronary artery bypass grafting (signs and symptoms of myocardial infarction with cTn values >10×99^{th} percentile URL).

In some embodiments, the patient receiving the antibody or antigen binding fragment that binds to ticagrelor or a metabolite or derivative thereof is in need of CABG. In some embodiments, the patient receiving the antibody or antigen binding fragment that binds to ticagrelor or a metabolite or derivative thereof is undergoing CABG. In the 18,000-patient clinical trial, Platelet Inhibition and Patient Outcomes, or PLATO, conducted by AstraZeneca, approximately 9% of patients on ticagrelor developed major bleeding that was related to procedures like CABG. Wallentin, L., et al. (2009). New England Journal of Medicine, 361(11), 1045-1057. Although the trial protocol recommended that patients who needed CABG stop taking ticagrelor for one to three days prior to start of surgery, nearly half of all ticagrelor patients needed surgery urgently and could not wait the up to three days for ticagrelor's effect to dissipate so normal blood clotting could be restored. Overall, up to 80% of CABG patients in the trial suffered a major or life-threatening bleeding event related to the surgery, and for those who needed urgent surgery and could not wait three days for ticagrelor to wash out, approximately 50% experienced a fatal or life-threatening bleeding event (Held et al., 2011). While some of this risk was likely associated with patients' underlying conditions, the overall bleeding risk is significantly increased by antiplatelet drugs, and the current US prescribing information for ticagrelor suggests suspension of ticagrelor treatment for five days prior to start of surgery. In some embodiments, the CABG is off-pump CABG, minimally invasive CABG, robot-assisted CABG, and/or hybrid procedure.

In some embodiments, the patient receiving the antibody or antigen binding fragment that binds to ticagrelor or a metabolite or derivative thereof has or is at risk of acute coronary syndrome. In some embodiments, the antibody or antigen-binding fragment thereof is administered to the patient during surgery, in a perioperative setting, or during an invasive procedure. In some embodiments, the antibody or antigen-binding fragment thereof is administered in the following schedule: 6 g infused over 10 minutes, followed by 6 g over 4 hours, followed by 6 g over 12 hours. In some embodiments, the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, administration of the antibody or antigen-binding fragment thereof results in improved percent inhibition of PRU in the patient.

In some embodiments, the patient receiving the antibody or antigen binding fragment that binds to ticagrelor or a metabolite or derivative thereof is being treated for intracranial hemorrhage, gastrointestinal bleeding, intra-articular bleeding, pericardial bleeding, or intramuscular bleeding with compartment syndrome. In some embodiments, intracranial hemorrhage is any bleeding inside the cranium. In some embodiments, intracranial hemorrhage is epidural hematoma, subdural hematoma, subarachnoid hemorrhage, intraventricular hemorrhage, intracerebral bleeding.

In some embodiments, the antibody or antigen-binding fragment thereof is administered to the patient during surgery, in a perioperative setting, or during an invasive procedure. In some embodiments, the patient is elderly, has underlying cardiac or pulmonary or metabolic disease, and/or has limited cardiac reserve. In some embodiments, the antibody or antigen-binding fragment thereof is administered in the following schedule: 6 g infused over 10 minutes, followed by 6 g over 4 hours, followed by 6 g over 12 hours. In some embodiments, the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, administration of the antibody or antigen-binding fragment thereof results in improved percent inhibition of PRU in the patient.

In some embodiments, the patient receiving the antibody or antigen binding fragment that binds to ticagrelor or a metabolite or derivative thereof has pulmonary or metabolic disease, and/or has limited cardiac reserve. In some embodiments, the patient receiving the antibody or antigen binding fragment that binds to ticagrelor or a metabolite or derivative thereof is elderly and/or fragile and/or has underlying cardiac disease. In some embodiments, the patient receiving the antibody or antigen binding fragment that binds to ticagrelor or a metabolite or derivative thereof is elderly. In some embodiments, an elderly patient is a patient who is 65 to 80 years old. In some embodiments, the age of the patient receiving the antibody or antigen binding fragment that binds to ticagrelor or a metabolite or derivative thereof is about 50 or older. In some embodiments, the age of the patient receiving the antibody or antigen binding fragment that binds to ticagrelor or a metabolite or derivative thereof is about 55 or older. In some embodiments, the age of the patient receiving the antibody or antigen binding fragment that binds to ticagrelor or a metabolite or derivative thereof is about 60 years or older. In some embodiments, the age of the patient is about 65 years or older. In some embodiments, the age of the patient is about 67 years or older. In some embodiments, the age of the patient is about 70 years or older. In some embodiments, the age of the patient is between about 60 to about 65 years old. In some embodiments, the age of the patient is between about 60 to about 70 years old. In some embodiments, the age of the patient is between about 60 to about 80 years old. In some embodiments, the age of the patient is between about 50 to about 80 years old. In some embodiments, the age of the patient is between about 50 to about 100 years old. In some embodiments, the age of the patient is between about 55 to about 100 years old. In some embodiments, the age of the patient is between about 60 to about 100 years old. In some embodiments, the age of the patient is between about 65 to about 100 years old. In some embodiments, the age of the patient is between about 50 to about 95 years old. In some embodiments, the age of the patient is between about 55 to about 95 years old. In some embodiments, the age of the patient is between about 60 to about 95 years old. In some embodiments, the age of the patient is between about 65 to about 95 years old. In some embodiments, the age of the patient is between about 55 to about 80 years old. In some embodiments, the age of the patient is between about 55 to about 75 years old. In some embodiments, the patient has underlying cardiac or pulmonary or metabolic disease, and/or has limited cardiac reserve. In some embodiments, the patient receiving the antibody or antigen binding fragment that binds to ticagrelor or a metabolite or derivative thereof is fragile. In some embodiments, a fragile patient is one who is elderly and/or has underlying cardiac or pulmonary or metabolic disease, limited cardiac reserve, cancer, peripheral and/or central nervous system disorders, gastrointestinal diseases, musculoskeletal diseases, and/or nutritional disorders, and/or who has had recent surgery or invasive procedure. In some embodiments, the recent surgery or invasive procedure is within about 0 days to about 1 day, about 1 day to about 3 day, about 3 days to about 7 days, about 7 days to 14 days, or about 14 days to 30 days. In some embodiments, the antibody or antigen-binding fragment thereof is administered to the patient during surgery, in a perioperative setting, or during an invasive procedure. In some embodiments, the antibody or antigen-binding fragment thereof is administered in the following schedule: 6 g infused over 10 minutes, followed by 6 g over 4 hours, followed by 6 g over 12 hours. In some embodiments, the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, administration of the antibody or antigen-binding fragment thereof results in improved percent inhibition of PRU in the patient.

In some embodiments, prior to administration of the antibody or antigen-binding fragment thereof that binds to ticagrelor or a metabolite or derivative thereof, the patient has PRU of between about 180 to about 230. In some embodiments, prior to administration of the antibody or antigen-binding fragment thereof, the patient has PRU of lower than about 180. In some embodiments, prior to administration of the antibody or antigen-binding fragment thereof, the patient has PRU of about 20 to 180 PRU. In some embodiments, prior to administration of the antibody or antigen-binding fragment thereof, the patient has PRU of about 20 to 30 PRU. In some embodiments, prior to administration of the antibody or antigen-binding fragment thereof, the patient has PRU of about 30 to 40 PRU. In some embodiments, prior to administration of the antibody or antigen-binding fragment thereof, the patient has PRU of about 40 to 50 PRU. In some embodiments, prior to administration of the antibody or antigen-binding fragment thereof, the patient has PRU of about 50 to 60 PRU. In some embodiments, prior to administration of the antibody or antigen-binding fragment thereof, the patient has PRU of about 60 to 70 PRU. In some embodiments, prior to administration of the antibody or antigen-binding fragment thereof, the patient has PRU of about 70 to 80 PRU. In some embodiments, prior to administration of the antibody or antigen-binding fragment thereof, the patient has PRU of about 80 to 90 PRU. In some embodiments, prior to administration of the antibody or antigen-binding fragment thereof, the patient has PRU of about 90 to 100 PRU. In some embodiments, prior to administration of the antibody or antigen-binding fragment thereof, the patient has PRU of about 100 to 110 PRU. In some embodiments, prior to administration of the antibody or antigen-binding fragment thereof, the patient has PRU of about 110 to 120 PRU. In some embodiments, prior to administration of the antibody or antigen-binding fragment thereof, the patient has PRU of about 120 to 130 PRU. In some embodiments, prior to administration of the antibody or antigen-binding fragment thereof, the patient has PRU of about 130 to 140 PRU. In some embodiments, prior to administration of the antibody or antigen-binding fragment thereof, the patient has PRU of about 140 to 150 PRU. In some embodiments, prior to administration of the antibody or antigen-binding fragment thereof, the patient has PRU of about 150 to 160 PRU. In some embodiments, prior to administration of the antibody or antigen-binding fragment thereof, the patient has PRU of about 160 to 170 PRU. In some embodiments, prior to administration of the antibody or antigen-binding fragment thereof, the patient has PRU of about 170 to 180 PRU. In some embodiments, PRU is not measured before administering the antibody or antigen-binding fragment thereof. In some embodiments, the antibody or antigen-binding fragment thereof is administered to the patient during surgery, in a perioperative setting, or during an invasive procedure. In some embodiments, administration of the antibody or antigen-binding fragment thereof results in improved percent inhibition of PRU in the patient. In some embodiments, the Fab is bentracimab. In some embodiments, the antibody or antigen-binding fragment thereof is administered to the patient during surgery, in a perioperative setting, or during an invasive procedure. In some embodiments, the antibody or antigen-binding fragment thereof is administered to the patient, wherein the patient is elderly, has underlying cardiac or pulmonary or metabolic disease, and/or has limited cardiac reserve. In some embodiments, the antibody or antigen-binding fragment thereof is administered in the following schedule: 6 g infused over 10 minutes, followed by 6 g over 4 hours, followed by 6 g over 12 hours.

In some embodiments, the patient receiving the antibody or antigen binding fragment that binds to ticagrelor or a metabolite or derivative thereof is in need of surgery or an invasive procedure known to be associated with a risk of significant bleeding. In some embodiments, the surgery or invasive procedure is cardiac surgery, gastrointestinal surgery, neurosurgery, or major orthopedic surgery. In some embodiments, the surgery or invasive procedure may have an adverse procedural outcome if hemostasis is impaired. In some embodiments, the antibody or antigen binding fragment that binds to ticagrelor or a metabolite or derivative thereof is administered to the patient during surgery. In some embodiments, the antibody or antigen-binding fragment thereof is administered to the patient during surgery, in a perioperative setting, or during an invasive procedure. In some embodiments, the antibody or antigen-binding fragment thereof is administered in the following schedule: 6 g infused over 10 minutes, followed by 6 g over 4 hours, followed by 6 g over 12 hours. In some embodiments, the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, administration of the antibody or antigen-binding fragment thereof results in improved percent inhibition of PRU in the patient.

In some embodiments, prior to administration of the antibody or antigen-binding fragment thereof that binds to ticagrelor or a metabolite or derivative thereof, the patient experiences major bleeding. In some embodiments, the patient is at risk of major bleeding. In some embodiments, the major bleeding is related to surgery or invasive procedure. In some embodiments, the major bleeding is intracranial hemorrhage including intracerebral bleeding, subdural hematoma, subarachnoid hematoma, and intraventricular bleeding. In some embodiments, the bleeding is intracranial hemorrhage. In some embodiments, intracranial hemorrhage is any bleeding inside the cranium. In some embodiments, intracranial hemorrhage is epidural hematoma, subdural hematoma, subarachnoid hemorrhage, intraventricular hemorrhage, or intracerebral bleeding. In some embodiments, the bleeding is secondary to trauma. In some embodiments, the bleeding is secondary to trauma. In some embodiments, the bleeding is caused or induced by trauma.

In some embodiments, the bleeding is spontaneous. In some embodiments, the patient has systolic blood pressure <90 mm Hg. In some embodiments, the patient has systolic blood pressure 40 mm Hg to 90 mm Hg. In some embodiments, the patient has systolic blood pressure 40 mm Hg to 50 mm Hg. In some embodiments, the patient has systolic blood pressure 50 mm Hg to 60 mm Hg. In some embodiments, the patient has systolic blood pressure 60 mm Hg to 70 mm Hg. In some embodiments, the patient has systolic blood pressure 70 mm Hg to 80 mm Hg. In some embodiments, the patient has systolic blood pressure 80 mm Hg to 90 mm Hg. In some embodiments, the bleeding is intracranial, intraspinal, intraocular, retroperitoneal, intra-articular, pericardial, or an intramuscular bleed with compartment syndrome. In some embodiments, the patient has a corrected hemoglobin level <8.0 g/dL, the patient has a fall in hemoglobin level of ≥2.0 g/dL (1.24 mmol/L) from a known baseline, and/or the patient has a transfusion of 2 or more units of packed red blood cells (PRBC). In some embodiments, the patient has a corrected hemoglobin level 0 g/dL to 8.0 g/dL. The patient has a fall in hemoglobin level of 2.0 g/dL to 18 g/dL from a known baseline. The patient has a fall in hemoglobin level of 2.0 g/dL to 9 g/dL from a known baseline. The patient has a fall in hemoglobin level of 2.0 g/dL to 5 g/dL from a known baseline. The patient has a fall in hemoglobin level of 2.0 g/dL to 4 g/dL from a known baseline. The patient has a fall in hemoglobin level of 2.0 g/dL to 3 g/dL from a known baseline. In some embodiments, the patient has a transfusion of 2 units to 10 units of packed red blood cells (PRBC). In some embodiments, the patient has a transfusion of 2 units to 3 units of PRBC. In some embodiments, the patient has a transfusion of 2 units to 4 units of PRBC. In some embodiments, the patient has a transfusion of 2 units to 5 units of PRBC. In some embodiments, the patient has a transfusion of 2 units to 6 units of PRBC. In some embodiments, the patient has a transfusion of 2 units to 7 units of PRBC. In some embodiments, the patient has a transfusion of 2 units to 8 units of PRBC. In some embodiments, the patient has a transfusion of 2 units to 9 units of PRBC. In some embodiments, the antibody or antigen-binding fragment thereof is administered to the patient during surgery, in a perioperative setting, or during an invasive procedure. In some embodiments, the antibody or antigen-binding fragment thereof is administered in the following schedule: 6 g infused over 10 minutes, followed by 6 g over 4 hours, followed by 6 g over 12 hours. In some embodiments, the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, administration of the antibody or antigen-binding fragment thereof results in improved percent inhibition of PRU in the patient.

In some embodiments, prior to administration of the antibody or antigen-binding fragment thereof that binds to ticagrelor or a metabolite or derivative thereof, the patient receives chronic treatment with acetylsalicylic acid. In some embodiments, prior to administration of the antibody or antigen-binding fragment thereof that binds to ticagrelor or a metabolite or derivative thereof, the patient does not treatment with acetylsalicylic acid. In some embodiments, the dose of acetylsalicylic acid is ≤100 mg. In some embodiments, the dose of acetylsalicylic acid is ≤325 mg per day. In some embodiments, the dose of acetylsalicylic acid is between 0 mg and 325 mg. In some embodiments, the patient receives chronic treatment with acetylsalicylic acid.

In some embodiments, the patient receiving the antibody or antigen binding fragment that binds to ticagrelor or a metabolite or derivative thereof was previously treated with ticagrelor for about 15 to 70 days. In some embodiments, the patient was previously treated with ticagrelor for about 0 to 483 days. In some embodiments, the patient was previously treated with ticagrelor for about 15 to 240 days. In some embodiments, the patient was previously treated with ticagrelor for about 1 to 12 months. In some embodiments, the patient was previously treated with ticagrelor for about 1 to 3 months. In some embodiments, the patient was previously treated with ticagrelor for about 1 to 6 months. In some embodiments, the patient was previously treated with ticagrelor for about 1 to 9 months. In some embodiments, the patient was previously treated with ticagrelor for about 0 to 1 month. In some embodiments, the patient was previously treated with ticagrelor for about 1 to 2 years. In some embodiments, the patient was previously treated with ticagrelor for about 2 to 3 years. In some embodiments, the patient was previously treated with ticagrelor for about 3 to 4 years. In some embodiments, the patient was previously treated with ticagrelor for about 4 to 5 years. In some embodiments, the patient was previously treated with ticagrelor for about 5 to 6 years. In some embodiments, the patient was previously treated with ticagrelor for about 6 to 7 years. In some embodiments, the patient was previously treated with ticagrelor for about 7 to 8 years. In some embodiments, the patient was previously treated with ticagrelor for about 8 to 9 years. In some embodiments, the patient was previously treated with ticagrelor for about 9 to 10 years. In some embodiments, the patient was previously treated with ticagrelor for about 1 to 5 years. In some embodiments, the patient was previously treated with ticagrelor for about 5 to 10 years. In some embodiments, the patient was previously treated with ticagrelor for about 1 to 10 years. In some embodiments, the patient was previously treated with ticagrelor within about three days of the antibody or antigen-binding fragment thereof. In some embodiments, the patient was previously treated with ticagrelor within about one day of the antibody or antigen-binding fragment thereof. In some embodiments, the patient was previously treated with a daily dose of between about 60 mg and about 360 mg of ticagrelor. In some embodiments, the antibody or antigen-binding fragment thereof is administered to the patient during surgery, in a perioperative setting, or during an invasive procedure. In some embodiments, the antibody or antigen-binding fragment thereof is administered in the following schedule: 6 g infused over 10 minutes, followed by 6 g over 4 hours, followed by 6 g over 12 hours. In some embodiments, the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, administration of the antibody or antigen-binding fragment thereof results in improved percent inhibition of PRU in the patient.

In some embodiments, prior to administration of the antibody or antigen-binding fragment thereof that binds to ticagrelor or a metabolite or derivative thereof, the patient has uncontrolled major or life-threatening bleeding with signs or symptoms of hemodynamic compromise, e.g., systolic blood pressure <90 mm Hg and signs or symptoms of low cardiac output not otherwise explained. In some embodiments, the patient has systolic blood pressure <90 mm Hg. In some embodiments, the patient has systolic blood pressure 40 mm Hg to 90 mm Hg. In some embodiments, the patient has systolic blood pressure 40 mm Hg to 50 mm Hg. In some embodiments, the patient has systolic blood pressure 50 mm Hg to 60 mm Hg. In some embodiments, the patient has systolic blood pressure 60 mm Hg to 70 mm Hg. In some embodiments, the patient has systolic blood pressure 70 mm Hg to 80 mm Hg. In some embodiments, the patient has systolic blood pressure 80 mm Hg to 90 mm Hg. In some embodiments, prior to administration of the antibody or antigen-binding fragment thereof, the patient has uncontrolled major or life-threatening bleeding in a critical organ or closed space, such as intracranial, intraspinal, intraocular, retroperitoneal, intra-articular, pericardial, or intramuscular bleed with compartment syndrome. In some embodiments, prior to administration of the antibody or antigen-binding fragment thereof, the patient has uncontrolled bleeding associated with a corrected hemoglobin level <8.0 g/dL, a fall in hemoglobin level of ≥2.0 g/dL (1.24 mmol/L) from a known baseline, or transfusion of 2 or more units of packed red blood cells (PRBC). In some embodiments, the patient has a corrected hemoglobin level 0 g/dL to 8.0 g/dL. The patient has a fall in hemoglobin level of 2.0 g/dL to 18 g/dL from a known baseline. The patient has a fall in hemoglobin level of 2.0 g/dL to 9 g/dL from a known baseline. The patient has a fall in hemoglobin level of 2.0 g/dL to 5 g/dL from a known baseline. The patient has a fall in hemoglobin level of 2.0 g/dL to 4 g/dL from a known baseline. The patient has a fall in hemoglobin level of 2.0 g/dL to 3 g/dL from a known baseline. In some embodiments, the patient has a transfusion of 2 units to 10 units of packed red blood cells (PRBC). In some embodiments, the patient has a transfusion of 2 units to 3 units of PRBC. In some embodiments, the patient has a transfusion of 2 units to 4 units of PRBC. In some embodiments, the patient has a transfusion of 2 units to 5 units of PRBC. In some embodiments, the patient has a transfusion of 2 units to 6 units of PRBC. In some embodiments, the patient has a transfusion of 2 units to 7 units of PRBC. In some embodiments, the patient has a transfusion of 2 units to 8 units of PRBC. In some embodiments, the patient has a transfusion of 2 units to 9 units of PRBC. In some embodiments, the uncontrolled bleeding is visible, uncontrolled bleeding. In some embodiments, the antibody or antigen-binding fragment thereof is administered to the patient during surgery, in a perioperative setting, or during an invasive procedure. In some embodiments, the antibody or antigen-binding fragment thereof is administered in the following schedule: 6 g infused over 10 minutes, followed by 6 g over 4 hours, followed by 6 g over 12 hours. In some embodiments, the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, administration of the antibody or antigen-binding fragment thereof results in improved percent inhibition of PRU in the patient.

In some embodiments, prior to administration of the antibody or antigen-binding fragment thereof that binds to ticagrelor or a metabolite or derivative thereof, the patient requires urgent surgery or invasive procedure known to be associated with a risk of significant bleeding (such as cardiac surgery, gastrointestinal surgery, neurosurgery, or major orthopedic surgery). In some embodiments, prior to treatment according to the methods disclosed herein, the patient requires urgent surgery or invasive procedure that may have an adverse procedural outcome if hemostasis was impaired (such as neurological, spinal, ophthalmological, urological, or orthopedic surgery). In some embodiments, prior to treatment according to the methods disclosed herein, the patient is at risk of experiencing life-threatening events, such as, shock, myocardial infarction, or stroke, if significant intraoperative or postoperative bleeding occurs (such as in elderly patients or patients with underlying cardiac or pulmonary disease who have limited cardiopulmonary reserve). In some embodiments, the antibody or antigen-binding fragment thereof is administered in the following schedule: 6 g infused over 10 minutes, followed by 6 g over 4 hours, followed by 6 g over 12 hours. In some embodiments, the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, administration of the antibody or antigen-binding fragment thereof results in improved percent inhibition of PRU in the patient.

In some embodiments, prior to administration of the antibody or antigen-binding fragment thereof that binds to ticagrelor or a metabolite or derivative thereof, the patient does not have stable or non-acute conditions with low hemoglobin due to chronic, low-grade gastrointestinal bleeding. In some embodiments, prior to administration of the antibody or antigen-binding fragment thereof that binds to ticagrelor or a metabolite or derivative thereof, the patient does not have stable, remote, or asymptomatic intracranial hemorrhage. In some embodiments, prior to administration of the antibody or antigen-binding fragment thereof that binds to ticagrelor or a metabolite or derivative thereof, the patient is not considered to be clinically unsalvageable. In some embodiments, the patient does not have end-stage cancer or overwhelming sepsis. In some embodiments, prior to administration of the antibody or antigen-binding fragment thereof that binds to ticagrelor or a metabolite or derivative thereof, the patient has not received clopidogrel, prasugrel, or ticlopidine within 5 days of bentracimab administration. In some embodiments, prior to administration of the antibody or antigen-binding fragment thereof that binds to ticagrelor or a metabolite or derivative thereof, the patient has not received antiplatelet GPIIb/IIIa inhibitors, or cangrelor within 5 half-lives of the start of administration of the antibody or antigen-binding fragment thereof. In some embodiments, prior to administration of the antibody or antigen-binding fragment thereof that binds to ticagrelor or a metabolite or derivative thereof, the patient has not received warfarin, dabigatran, rivaroxaban, apixaban, or edoxaban. In some embodiments, prior to administration of the antibody or antigen-binding fragment thereof that binds to ticagrelor or a metabolite or derivative thereof, the patient has not received warfarin, dabigatran, rivaroxaban, apixaban, or edoxaban within 5 half-lives of the start of administration of bentracimab. In some embodiments, prior to administration of the antibody or antigen-binding fragment thereof that binds to ticagrelor or a metabolite or derivative thereof, the patient has not received vitamin K, prothrombin complex concentrate, recombinant factor VIIa, idarucizumab, or andexanet-alfa (coagulation factor Xa (recombinant), inactivated-zhzo) within 5 days of the start of administration of the antibody or antigen-binding fragment thereof. In some embodiments, the antibody or antigen-binding fragment thereof is administered to the patient during surgery, in a perioperative setting, or during an invasive procedure. In some embodiments, the antibody or antigen-binding fragment thereof is administered in the following schedule: 6 g infused over 10 minutes, followed by 6 g over 4 hours, followed by 6 g over 12 hours. In some embodiments, the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, administration of the antibody or antigen-binding fragment thereof results in improved percent inhibition of PRU in the patient.

In some embodiments, the patient has kidney disease. In some embodiments, the kidney disease is mild, severe, mild-moderate, moderate-severe, or severe. In some embodiments, the kidney disease is end-stage renal disease In some embodiments, the patient does not have kidney disease. In some embodiments, the patient has renal impairment. In some embodiments, the renal impairment is mild renal impairment or moderate renal impairment. In some embodiments, the antibody or antigen-binding fragment thereof is administered to the patient during surgery, in a perioperative setting, or during an invasive procedure. In some embodiments, the antibody or antigen-binding fragment thereof is administered in the following schedule: 6 g infused over 10 minutes, followed by 6 g over 4 hours, followed by 6 g over 12 hours. In some embodiments, the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, administration of the antibody or antigen-binding fragment thereof results in improved percent inhibition of PRU in the patient.

In some embodiments, bentracimab is indicated in patients treated with ticagrelor when reversal of the antiplatelet effects of ticagrelor and its active metabolite is needed, In some embodiments, the patients are requiring nondeferrable surgery/invasive procedure. In some embodiments, the patients are experiencing major bleeding.

### Endpoints and Safety

In some embodiments, the methods provided herein comprising administering an antibody or antigen binding fragment that binds to ticagrelor or a metabolite or derivative thereof to a patient result in improving percent inhibition of PRU in the patient. In some embodiments, administering an antibody or antigen binding fragment that binds to ticagrelor or a metabolite or derivative thereof to a population of patients results in improving mean percent inhibition of PRU in the population of patients. In some embodiments, the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, administering the antibody or antigen binding fragment that binds to ticagrelor or a metabolite or derivative thereof to a population of patients results in a mean minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding. In some embodiments, a population of patients is the population of patients enrolled in a clinical trial. Improving percent inhibition of PRU is decreasing percent inhibition of PRU compared to baseline, i.e., prior to administration of the antibody or antigen-binding fragment thereof that binds to ticagrelor or a metabolite or derivative thereof. In some embodiments, administering the antibody or antigen-binding fragment thereof to the patient results in a decrease in percent inhibition of PRU in the patient compared to baseline. In some embodiments, the mean percent inhibition of PRU over 4 hours after starting administration of bentracimab is decreased compared to the percent inhibition of PRU observed prior to start of administration. In an example, a patient has improved percent inhibition of PRU if the patient has PRU of 50 at baseline, i.e., prior to start of bentracimab administration, and the minimum percent inhibition of PRU observed over a period of 4 hours after starting administration of bentracimab is −70. In some embodiments, percent inhibition of PRU is determined by calculating 100×(180−[PRUₜᵣₜ])/180, wherein PRUₜᵣₜ refers to the PRU value measured posttreatment with bentracimab. In an example, a patient who has a PRU posttreatment with bentracimab of 300 would have a percent inhibition of PRU of 100×(180−300)/180=−66.

In some embodiments, the methods provided herein comprising administering an antibody or antigen binding fragment that binds to ticagrelor or a metabolite or derivative thereof to a patient resulting in improved platelet function. In some embodiments, administration of the antibody or antigen binding fragment that binds to ticagrelor or a metabolite or derivative thereof results in platelet function returns to normal within 5-10 minutes of administration of the antibody or antigen binding fragment thereof. In some embodiments, administration of the antibody or antigen binding fragment that binds to ticagrelor or a metabolite or derivative thereof results in platelet function returns to normal within 5-6, 5-7, 7-8, or 5-9 minutes of administration of the antibody or antigen binding fragment thereof. In some embodiments, administration of the antibody or antigen binding fragment that binds to ticagrelor or a metabolite or derivative thereof results in platelet function returns to normal within 6-10, 7-10, 8-10, or 9-10 minutes of administration of the antibody or antigen binding fragment thereof. In some embodiments, normal platelet function is quantified by PRU.

In some embodiments, normal platelet function results in effective homeostasis. In some embodiments, administration of the antibody or antigen binding fragment that binds to ticagrelor or a metabolite or derivative thereof results in effective homeostasis within 5-10 minutes of administration of the antibody or antigen binding fragment thereof.

Methods for measuring PRU are known in the art, e.g., VerifyNow-P2Y₁₂ Assay/VerifyNow™ PRUTest. The VerifyNow™-P2Y₁₂ Assay/VerifyNow™ PRUTest is a rapid test that uses ADP to stimulate platelets in the presence of PGE1 (Prostaglandin E1) and which inhibits activation downstream of a second ADP receptor P2Y₁, making the assay more sensitive and specific for the activity of the P2Y₁₂ receptor and of drugs that bind to the P2Y₁₂ receptor. The assay system reagent is designed to specifically measure P2Y₁₂-mediated platelet aggregation. In some embodiments, ticagrelor reversal is based on a measurement of PRU. In some embodiments, PRU is the reduction of percent (%) inhibition of platelet function as measured by the VerifyNow™ PRUTest™ assay within 4 hours of initiation of bentracimab. For the VerifyNow™ PRUTest, a PRU of 10-180 generally suggests a decreased platelet reactivity to P2Y₁₂ inhibitor, whereas >180-376 PRU suggestive of uninhibited platelet reactivity. In some embodiments, blood sampling for platelet function tests is collected within 1 hour of study drug administration. In some embodiments, PRU assay is performed immediately, e.g., within 2 hours, after the blood sample is drawn. In some embodiments, PRU is assayed by a turbidimetric based optical detection system. In some embodiments, the turbidimetric based optical detection system measures platelet-induced aggregation. In some embodiments, the PRU assay measures platelet aggregation mediated by P2Y₁₂ receptor blockade. In some embodiments, the PRU assay detects the ability of activated platelets to bind fibrinogen. In some embodiments, the PRU assay comprises measuring fibrinogen-coated microparticles aggregating in whole blood in proportion to the number of expressed platelet GP IIb/IIIa receptors. In some embodiments, the results of the PRU assay are reported as P2Y₁₂ Reaction Units (PRU) based on the rate and extent of aggregation. In some embodiments, the PRU result reflects the amount of P2Y₁₂ receptor-mediated aggregation specific to platelets. In some embodiments, the PRU result calculates the rate and extent of platelet aggregation recorded in the channel containing the platelet agonist, ADP. In some embodiments, the PRU result is not influenced by non-specific platelet aggregation mediated through P2Y₁. In some embodiments, the PRU assay uses prostaglandin E₁(PGE₁) in addition to ADP to make the test more sensitive and specific for the effects of ADP mediated by the P2Y₁₂ receptor. In some embodiments, the antibody or antigen-binding fragment thereof is administered to the patient during surgery, in a perioperative setting, or during an invasive procedure. In some embodiments, the antibody or antigen-binding fragment thereof is administered to the patient, wherein the patient is elderly, has underlying cardiac or pulmonary or metabolic disease, and/or has limited cardiac reserve. In some embodiments, the antibody or antigen-binding fragment thereof is administered in the following schedule: 6 g infused over 10 minutes, followed by 6 g over 4 hours, followed by 6 g over 12 hours.

In some embodiments, an ELISA-based assay is used to assess ticagrelor reversal after administering the antibody or antigen-binding fragment thereof that binds to ticagrelor or a metabolite or derivative thereof. In some embodiments, vasodilator stimulated phosphoprotein (VASP) phosphorylation assay is used to assess ticagrelor reversal. The VASP assay a standardized flow cytometric test. In some embodiments, the VASP assay does not rely on co-activation of the P2Y₁ receptor by ADP. In some embodiments, the VASP assay comprises incubating samples of citrate-anticoagulated whole blood with in vitro with prostaglandin E₁ (PGE₁), with or without ADP, before fixation. In some embodiments, the VASP assay further comprises permeabilizing the platelets and labeled them with a primary monoclonal antibody against serine 239-phosphorylated VASP (clone 16C2) or its isotype, followed by a secondary fluorescein isothiocyanate-conjugated polyclonal goat anti-mouse antibody. In some embodiments, the samples are analyzed in a flow cytometer. In some embodiments, the platelet population is identified by its forward and side scatter distribution. In some embodiments, the extent of VASP phosphorylation is measured by geometric mean fluorescence intensity (MFI) values in the presence of PGE₁ without (T1) or with ADP (T2). In some embodiments, the platelet reactivity index (PRI) is calculated according to the following formula: PRI %=T1 PGE₁−T2 PGE+ADP/T₁ PGE₁×100. In some embodiments, PRI represents the mean percentage platelet reactivity. In some embodiments, PRI is inversely correlated with platelet inhibition by P2Y₁₂ receptor blockers.

In some embodiments, the turbidimetric based optical detection assay is used for primary assessment of ticagrelor reversal after administration of the antibody or antigen-binding fragment thereof that binds to ticagrelor or a metabolite or derivative thereof and the VASP assay is used for secondary assessment of ticagrelor reversal.

In some embodiments, the patient has reduced minimum percent inhibition of P2Y₁₂ reaction units (PRU) in 4 hours after starting administration of the antibody or antigen-binding fragment thereof that binds to ticagrelor or a metabolite or derivative thereof compared to the percent inhibition of PRU before administration, thereby reversing ticagrelor-enhanced bleeding. In some embodiments, percent inhibition of PRU is determined by calculating 100×(180−[PRUₜᵣₜ])/180. In some embodiments, the lower the minimum percent inhibition of P2Y₁₂ reaction units (PRU) over 4 hours, the greater the therapeutic efficacy of reversing ticagrelor-enhanced bleeding. In some embodiments, the greater the difference between the minimum percent inhibition of PRU in 4 hours after starting administration of bentracimab compared to the percent inhibition of PRU at baseline, i.e., prior to treatment with bentracimab, the greater the therapeutic efficacy of reversing ticagrelor-enhanced bleeding.

In some embodiments, minimum percent inhibition of PRU in 4 hours after starting administration of the antibody or antigen-binding fragment thereof that binds to ticagrelor or a metabolite or derivative thereof, as described herein, refers to minimum inhibition of PRU over 4 hours after starting administration to a 1) a patient with uncontrolled major or life-threatening bleeding; or 2) a patient in need of surgery or an invasive procedure. In some embodiments, the patient with uncontrolled major or life-threatening bleeding starts treatment with bentracimab during surgery.

In some embodiments, administering the antibody or antigen-binding fragment thereof that binds to ticagrelor or a metabolite or derivative thereof to the patient in need of reversal of ticagrelor-enhanced bleeding results in the patient having a minimum inhibition of PRU over 4 hours after starting administration of between about −76 and about −67. In some embodiments, administering the antibody or antigen-binding fragment thereof to the population of patients results in a mean minimum inhibition of PRU over 4 hours after starting administration of between about −76 and about −67 in the population of patients. In some embodiments, the baseline percent inhibition of PRU in the patient is between about 45 to about 57. In some embodiments, the mean baseline percent inhibition of PRU in the population of patients is between about 45 and about 57. In some embodiments, the difference between minimum percent inhibition of P2Y₁₂ reaction units (PRU) over 4 hours after starting administration of the antibody or antigen-binding fragment thereof and the baseline percent inhibition of PRU before administration in a patient is between about −129 and about −115, where patient has a minimum percent inhibition of P2Y₁₂ reaction units (PRU) over 4 hours after starting administration of the antibody or antigen-binding fragment thereof that is less than the percent inhibition of PRU before administration. In some embodiments, the mean difference between minimum percent inhibition of P2Y₁₂ reaction units (PRU) over 4 hours after starting administration of the antibody or antigen-binding fragment thereof and the baseline percent inhibition of PRU before administration in a population of patients is between about −129 and about −115, where the population of patients has a minimum percent inhibition of P2Y₁₂ reaction units (PRU) over 4 hours after starting administration of the antibody or antigen-binding fragment thereof that is less than the percent inhibition of PRU before administration.

### 1) Surgery

In some embodiments, administering the antibody or antigen-binding fragment thereof that binds to ticagrelor or a metabolite or derivative thereof to the patient in need of reversal of ticagrelor-enhanced bleeding results the patient having a minimum percent inhibition of P2Y₁₂ reaction units (PRU) over 4 hours after starting administration of the antibody or antigen-binding fragment thereof that is less than the percent inhibition of PRU before administration, wherein the patient is in need of surgery or an invasive procedure. In some embodiments, the patient is in need of surgery or an invasive procedure known to be associated with a risk of significant bleeding. In some embodiments, administering the antibody or antigen-binding fragment thereof to the patient results in a minimum percent inhibition of PRU over 4 hours after starting administration of between about −78 and about −68 in the patient. In some embodiments, administering the antibody or antigen-binding fragment thereof to a population of patients results in a mean minimum inhibition of PRU over 4 hours after starting administration of between about −78 and about −68 in the population of patients. In some embodiments, the baseline percent inhibition of PRU in the patient is between about 45 to about 58. In some embodiments, the mean baseline percent inhibition of PRU in the population of patients is between about 45 and about 58. In some embodiments, the difference between minimum percent inhibition of P2Y₁₂ reaction units (PRU) over 4 hours after starting administration of the antibody or antigen-binding fragment thereof and the baseline percent inhibition of PRU before administration in a patient is between −132 and about −117, where patient has a minimum percent inhibition of P2Y₁₂ reaction units (PRU) over 4 hours after starting administration of the antibody or antigen-binding fragment thereof that is less than the percent inhibition of PRU before administration. In some embodiments, the mean difference between minimum percent inhibition of P2Y₁₂ reaction units (PRU) over 4 hours after starting administration of the antibody or antigen-binding fragment thereof and the baseline percent inhibition of PRU before administration in a population of patients is between about −132 and about −117, where the population of patients has a minimum percent inhibition of P2Y₁₂ reaction units (PRU) over 4 hours after starting administration of the antibody or antigen-binding fragment thereof that is less than the percent inhibition of PRU before administration.

In some embodiments, the methods comprise performing a surgery or invasive procedure on the patient and administering the antibody or antigen-binding fragment thereof that binds to ticagrelor or a metabolite or derivative thereof to the patient in need of reversal of ticagrelor-enhanced bleeding results the patient having a minimum percent inhibition of P2Y₁₂ reaction units (PRU) over 4 hours after starting administration of the antibody or antigen-binding fragment thereof that is less than the percent inhibition of PRU before administration. In some embodiments, the patient is in need of surgery or an invasive procedure known to be associated with a risk of significant bleeding. In some embodiments, administering the antibody or antigen-binding fragment thereof to the patient results in a minimum percent inhibition of PRU over 4 hours after starting administration of between about −78 and about −68 in the patient. In some embodiments, administering the antibody or antigen-binding fragment thereof to a population of patients results in a mean minimum inhibition of PRU over 4 hours after starting administration of between about −78 and about −68 in the population of patients. In some embodiments, the baseline percent inhibition of PRU in the patient is between about 45 to about 58. In some embodiments, the mean baseline percent inhibition of PRU in the population of patients is between about 45 and about 58. In some embodiments, the difference between minimum percent inhibition of P2Y₁₂ reaction units (PRU) over 4 hours after starting administration of the antibody or antigen-binding fragment thereof and the baseline percent inhibition of PRU before administration in a patient is between −132 and about −117, where patient has a minimum percent inhibition of P2Y₁₂ reaction units (PRU) over 4 hours after starting administration of the antibody or antigen-binding fragment thereof that is less than the percent inhibition of PRU before administration. In some embodiments, the mean difference between minimum percent inhibition of P2Y₁₂ reaction units (PRU) over 4 hours after starting administration of the antibody or antigen-binding fragment thereof and the baseline percent inhibition of PRU before administration in a population of patients is between about −132 and about −117, where the population of patients has a minimum percent inhibition of P2Y₁₂ reaction units (PRU) over 4 hours after starting administration of the antibody or antigen-binding fragment thereof that is less than the percent inhibition of PRU before administration.

In some embodiments, administering the antibody or antigen-binding fragment thereof that binds to ticagrelor or a metabolite or derivative thereof results in a reduced number of transfusions within 120 hours after start of urgent surgery received by a first patient (or population of patients) receiving the antibody or antigen-binding fragment thereof. In some embodiments, the number of transfusions within 120 hours after start of urgent surgery is reduced compared to number of transfusions within 120 hours after start of major bleeding received by a second patient or second population of patients who is not administered the antibody or antigen-binding fragment thereof, wherein both the first patient (or population of patients) and second patient (or population of patients) require urgent surgery. In some embodiments, the antibody or antigen-binding fragment thereof is bentracimab.

In some aspects, provided herein are methods of reversing ticagrelor-enhanced bleeding in a patient (or population of patients) comprising: i) administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to a population of patients, wherein the population of patients require urgent surgery, then ii) administering no more than a mean of 1 transfusion to the population of patients within 120 hours after start of urgent surgery in the population of patients requiring urgent surgery. In some embodiments, the antibody or antigen-binding fragment thereof is bentracimab.

In some embodiments, administering the antibody or antigen-binding fragment thereof that binds to ticagrelor or a metabolite or derivative thereof results in reduced risk of requiring surgery to treat bleeding within 48 hours after the start of urgent surgery in a first patient (or population of patients) receiving the antibody or antigen-binding fragment thereof. In some embodiments, the risk of requiring surgery to treat bleeding within 48 hours after the start of urgent surgery is reduced compared to risk of requiring surgery to treat bleeding within 48 hours after the start of urgent surgery in a second patient or second population of patients who is not administered the antibody or antigen-binding fragment thereof, wherein the first patient (or population of patients) and second patient (or population of patients) both require urgent surgery. In some embodiments, the antibody or antigen-binding fragment thereof is bentracimab.

In some embodiments, administration of the antibody or antigen-binding fragment thereof that binds to ticagrelor or a metabolite or derivative thereof to a population of patients results in a treatment-emergent adverse event related to the administration of the antibody or antigen-binding fragment thereof in less than about 0.5% of patients within the population of patients, wherein the treatment-emergent adverse event requires discontinuation of administration of the antibody or antigen-binding fragment thereof. In some embodiments, administration of the antibody or antigen-binding fragment thereof to a population of patients results in a treatment-emergent adverse event related to the administration of the antibody or antigen-binding fragment thereof resulting in death in less than about 5% of patients within the population of patients. In some embodiments, the population of patients requires urgent surgery. In some embodiments, the antibody or antigen-binding fragment thereof is bentracimab.

### 2) Major Bleeding

In some embodiments, administering the antibody or antigen-binding fragment thereof that binds to ticagrelor or a metabolite or derivative thereof to the patient in need of reversal of ticagrelor-enhanced bleeding results the patient having a minimum percent inhibition of P2Y₁₂ reaction units (PRU) over 4 hours after starting administration of the antibody or antigen-binding fragment thereof that is less than the percent inhibition of PRU before administration, wherein the patient has uncontrolled major or life-threatening bleeding. In some embodiments, the patient has uncontrolled major or life-threatening bleeding associated with surgery or an invasive procedure. In some embodiments, administering the antibody or antigen-binding fragment thereof to the patient results in a minimum percent inhibition of PRU over 4 hours after starting administration of between about −78 and about −56 in the patient. In some embodiments, administering the antibody or antigen-binding fragment thereof to a population of patients results in a mean minimum inhibition of PRU over 4 hours after starting administration of between about −78 and about −56 in the population of patients. In some embodiments, the baseline percent inhibition of PRU in the patient is between about 34 to about 62. In some embodiments, the mean baseline percent inhibition of PRU in the population of patients is between about 34 and about 62. In some embodiments, the difference between minimum percent inhibition of P2Y₁₂ reaction units (PRU) over 4 hours after starting administration of the antibody or antigen-binding fragment thereof and the baseline percent inhibition of PRU before administration in a patient is between about −130 and about −101, where patient has a minimum percent inhibition of P2Y₁₂ reaction units (PRU) over 4 hours after starting administration of the antibody or antigen-binding fragment thereof that is less than the percent inhibition of PRU before administration. In some embodiments, the mean difference between minimum percent inhibition of P2Y₁₂ reaction units (PRU) over 4 hours after starting administration of the antibody or antigen-binding fragment thereof and the baseline percent inhibition of PRU before administration in a population of patients is between about −130 and about −101, where the population of patients has a minimum percent inhibition of P2Y₁₂ reaction units (PRU) over 4 hours after starting administration of the antibody or antigen-binding fragment thereof that is less than the percent inhibition of PRU before administration.

In some embodiments, administering the antibody or antigen-binding fragment thereof that binds to ticagrelor or a metabolite or derivative thereof to the patient results in the patient having PRU of between about 180 and about 418. In some embodiments, administering the antibody or antigen-binding fragment thereof to the patient results in the patient having PRU of between about 180 and about 200. In some embodiments, administering the antibody or antigen-binding fragment thereof to the patient results in the patient having PRU of between about 180 and about 300. In some embodiments, administering the antibody or antigen-binding fragment thereof to the patient results in the patient having PRU of between about 180 and about 400. In some embodiments, administering the antibody or antigen-binding fragment thereof to the patient results in the patient having PRU of between about 200 and about 300. In some embodiments, administering the antibody or antigen-binding fragment thereof to the patient results in the patient having PRU of between about 300 and about 400. In some embodiments, administering the antibody or antigen-binding fragment thereof to the patient results in the patient having PRU of between about 200 and about 250. In some embodiments, administering the antibody or antigen-binding fragment thereof to the patient results in the patient having PRU of between about 250 and about 300. In some embodiments, administering the antibody or antigen-binding fragment thereof to the patient results in the patient having PRU of between about 300 and about 350. In some embodiments, administering the antibody or antigen-binding fragment thereof to the patient results in the patient having PRU of between about 350 and about 400. In some embodiments, administering the antibody or antigen-binding fragment thereof to a population of patients results in an increase in the mean PRU of the population of patients. In some embodiments, administering the antibody or antigen-binding fragment thereof to the population of patients results a mean PRU of between about 180 and about 200 in the population of patients. In some embodiments, administering the antibody or antigen-binding fragment thereof to the population of patients results a mean PRU of between about 180 and about 300 in the population of patients. In some embodiments, administering the antibody or antigen-binding fragment thereof to the population of patients results a mean PRU of between about 180 and about 400 in the population of patients. In some embodiments, administering the antibody or antigen-binding fragment thereof to the population of patients results a mean PRU of between about 200 and about 300 in the population of patients. In some embodiments, administering the antibody or antigen-binding fragment thereof to the population of patients results a mean PRU of between about 300 and about 400 in the population of patients. In some embodiments, administering the antibody or antigen-binding fragment thereof to the population of patients results a mean PRU of between about 200 and about 250 in the population of patients. In some embodiments, administering the antibody or antigen-binding fragment thereof to the population of patients results a mean PRU of between about 250 and about 300 in the population of patients. In some embodiments, administering the antibody or antigen-binding fragment thereof to the population of patients results a mean PRU of between about 300 and about 350 in the population of patients. In some embodiments, administering the antibody or antigen-binding fragment thereof to the population of patients results a mean PRU of between about 350 and about 400 in the population of patients.

In some embodiments, methods provided herein comprising administering an antibody or antigen binding fragment that binds to ticagrelor or a metabolite or derivative thereof to a patient result in improving PRU in the patient. Improving PRU is increasing PRU compared to baseline, i.e., prior to administration of the antibody or antigen-binding fragment thereof that binds to ticagrelor or a metabolite or derivative thereof. In some embodiments, methods provided herein comprising administering an antibody or antigen binding fragment that binds to ticagrelor or a metabolite or derivative thereof to a patient result in normalizing platelet function as assessed by PRU or PRI. In some embodiments, normalizing platelet function as assessed by PRU comprises increasing PRU to about 180 or higher. In some embodiments, normalizing platelet function as assessed by PRU comprises increasing PRU to about 180 to 240. In some embodiments, normalizing platelet function as assessed by PRU comprises increasing PRU to about 180 to 230. In some embodiments, normalizing platelet function as assessed by PRI comprises increasing PRI to about 80% or higher. In some embodiments, normalizing platelet function as assessed by PRI comprises increasing PRI to about 80% to 90%. In some embodiments, normalizing platelet function as assessed by PRI comprises increasing PRI to about 80% to 100%. In some embodiments, the normalization of platelet function occurs within about 5 to about 10 minutes and is sustained for at least about 24 hours after starting administration of the antibody or antigen binding fragment thereof. In some embodiments, the antibody or antigen binding fragment normalizes platelet function as assessed by PRU or PRI within about 5 minutes to 10 minutes after starting administration of the antibody or antigen binding fragment thereof. In some embodiments, the antibody or antigen binding fragment thereof results in normalization of platelet function in a patient within about 5 minutes to 10 minutes after starting administration of the antibody or antigen binding fragment thereof. In some embodiments, the antibody or antigen binding fragment thereof results in sustained normalization of platelet function in a patient for at least 24 hours after starting administration of the antibody or antigen binding fragment thereof. In some embodiments, the antibody or antigen binding fragment thereof results in sustained normalization of platelet function in a patient for at least 24 hours after starting administration of the antibody or antigen binding fragment thereof. In some embodiments, the antibody or antigen-binding fragment thereof is administered to the patient during surgery, in a perioperative setting, or during an invasive procedure. In some embodiments, the antibody or antigen-binding fragment thereof is administered in the following schedule: 6 g infused over 10 minutes, followed by 6 g over 4 hours, followed by 6 g over 12 hours.

In some embodiments, administering the antibody or antigen-binding fragment thereof that binds to ticagrelor or a metabolite or derivative thereof results in the patient achieving good or excellent homeostasis. In some embodiments, administering the antibody or antigen-binding fragment thereof that binds to ticagrelor or a metabolite or derivative thereof results in a population of patients achieving good or excellent homeostasis. In some embodiments, administering the antibody or antigen-binding fragment thereof that binds to ticagrelor or a metabolite or derivative thereof results in about 95% of a population of patients achieving good or excellent homeostasis. In some embodiments, administering the antibody or antigen-binding fragment thereof that binds to ticagrelor or a metabolite or derivative thereof results in about 90% to 98% of a population of patients achieving good or excellent homeostasis. In some embodiments, administering the antibody or antigen-binding fragment thereof to a population of patients requiring surgery or invasive procedure results in excellent or good hemostasis in about 100% of the population of patients. In some embodiments, administering the antibody or antigen-binding fragment thereof to a population of patients requiring surgery or invasive procedure results in excellent or good hemostasis in about 99% of the population of patients. In some embodiments, administering the antibody or antigen-binding fragment thereof to a population of patients requiring surgery or invasive procedure results in excellent or good hemostasis in about 98% of the population of patients. In some embodiments, administering the antibody or antigen-binding fragment thereof to a population of patients with uncontrolled major bleeding results in excellent or good hemostasis in at least about 95% of the population of patients. In some embodiments, administering the antibody or antigen-binding fragment thereof to a population of patients with uncontrolled major bleeding results in excellent or good hemostasis in about 80% of the population of patients. In some embodiments, administering the antibody or antigen-binding fragment thereof to a population of patients with uncontrolled major bleeding results in excellent or good hemostasis in between about 65% and 90% of the population of patients. In some embodiments, a population of patients is the population of patients enrolled in a clinical trial. In some embodiments, achievement of effective hemostasis is determined using standardized “effective hemostasis” bleeding scales. Table 2 shows the rating system for effective hemostasis in bleeding patients. Table 3 shows the rating system for effective hemostasis in surgical patents.

TABLE 2
Rating System for Effective Hemostasis in Bleeding Patients

| Hemostasis | Visible Bleeding | Non-Visible Bleeding |
|---|---|---|
| Excellent | Cessation of | Muscular/skeletal: pain relief or no increase in |
| (effective) | bleeding ≤1 hour | swelling or unequivocal improvement in objective |
| | after end of infusion | signs of bleeding ≤1 hour after the end of infusion; |
| | and no plasma, | and the condition has not deteriorated during the |
| | coagulation factor | 12-hour period after the end of infusion |
| | or blood products | Intracerebral hematoma (ICH): ≤20% increase in |
| | (excludes pRBCs).¹ | hematoma volume compared to baseline on a repeat |
| | | CT or MRI scan performed at both the 1 and 12 |
| | | hour post infusion time points |
| | | Subarachnoid bleed: ≤20% increase in maximum |
| | | thickness using the most dense area on the follow- |
| | | up vs baseline at both the 1 and 12 hour post |
| | | infusion time points |
| | | Subdural hematoma: ≤20% increase in maximum |
| | | thickness at both the 1 and 12 hour post infusion |
| | | assessments compared to baseline |
| | | Pericardial: No increase in the size of pericardial |
| | | effusion on repeat echocardiogram done within 12 |
| | | hours of the end of infusion |
| | | Intraspinal: No increase in the size of pericardial |
| | | effusion on repeat CT or MRI done within 12 hours |
| | | of the end of infusion |
| | | Non-visible bleeding not described above (e.g., |
| | | gastrointestinal, urinary): ≤10% decrease in both |
| | | corrected hemoglobin/hematocrit at 24 hours^{2, 3} |
| | | compared to baseline |
| Good | Cessation of bleeding | Muscular/skeletal: pain relief or no increase in |
| (effective) | between >1 and ≤4 | swelling or unequivocal improvement in objective |
| | hours after end of | signs of bleeding >1 and ≤4 hours after end of |
| | infusion and ≤2 | infusion; and the condition has not deteriorated |
| | units plasma, | during the 12-hour period after the end of infusion |
| | coagulation factor | ICH: >20% but ≤35% increase in hematoma volume |
| | or blood products | compared to baseline on a repeat CT or MRI scan |
| | (excludes pRBCs).⁴ | at +12-hour time point after the end of infusion |
| | | Subarachnoid bleed: >20% but <35% increase in |
| | | maximum thickness using the most dense area on |
| | | the follow-up at +12 h after end of infusion vs |
| | | baseline |
| | | Subdural hematoma: >20% but <35% increase in |
| | | maximum thickness at +12 h after end of infusion |
| | | compared to baseline |
| | | Pericardial: <10% increase in the size of pericardial |
| | | effusion on repeat echocardiogram done within 12 |
| | | hours of the end of infusion |
| | | Intraspinal: <10% increase in the size of pericardial |
| | | effusion on repeat CT or MRI done within 12 hours |
| | | of the end of infusion |
| | | Non-visible bleeding not described above (e.g., |
| | | gastrointestinal, urinary): >10% to ≤20% decrease |
| | | in both corrected hemoglobin/hematocrit at 24 hours |
| | | compared to baseline^{2, 3} |
| Poor/None | Cessation of | Muscular/skeletal: No improvement by 4 hours after |
| (not | bleeding >4 hours | end of infusion and/or condition has deteriorated |
| effective) | after end of the | during the 12-hour period |
| | infusion and/or >2 | ICH: >35% increase in hematoma volume on a CT |
| | units plasma, | or MRI compared to baseline on a repeat CT or MRI |
| | coagulation factor | scan at +12-hour time point after the end of infusion |
| | or blood products | Subarachnoid bleed: >35% increase in maximum |
| | (excludes pRBCs)⁵ | thickness using the most dense area on the follow- |
| | | up at +12 h after end of infusion vs baseline |
| | | Subdural hematoma: >35% increase in maximum |
| | | thickness at +12 h after end of infusion compared |
| | | to baseline |
| | | Pericardial: 10% or more increase in the size of |
| | | pericardial effusion on repeat echocardiogram done |
| | | within 12 hours of the end of infusion |
| | | Intraspinal: 10% or more increase in the size of |
| | | pericardial effusion on repeat CT or MRI done |
| | | within 12 hours of the end of infusion |
| | | Non-visible bleeding not described above (e.g., |
| | | gastrointestinal, urinary): >20% decrease in both |
| | | corrected hemoglobin/hematocrit^{2, 3} |

Additional factors

Any additional diagnostic data for a particular bleeding site (e.g., nasogastric tube,
ultrasound, GI endoscope, or CT scans) will be taken into account for the overall
assessment
Any uncontrolled bleeding that did not respond to bentracimab and was related to an
underlying disease will be taken into account for the overall assessment
Pain, swelling, and signs of bleeding are considered typical symptoms of
musculoskeletal bleeding and are expected to be present at baseline
Footnotes:
¹For all types of bleeding: no additional plasma, blood products (whole blood products not including packed red blood cells [PRBCs]) and/or coagulation factor products required after initial treatment with bentracimab
²The smallest percentage decrease in hemoglobin or hematocrit should be used to determine the efficacy rating of excellent, good, or poor/none. The net change is defined as the difference between the corrected hemoglobin or hematocrit value at baseline and 12 hours after infusion
³For the adjusted hemoglobin and hematocrit calculation, it will be assumed that for each unit of PRBC transfusion there is an increase of 1 g/dL in hemoglobin and a 3% increase in hematocrit
⁴For all types of bleeding, no more than two additional units of plasma or blood products and/or coagulation factor products required after initial treatment with bentracimab. “Blood products” include whole blood but not PRBCs.
⁵For all types of bleeding, more than two additional units of plasma or blood products and/or coagulation factor products required after initial treatment with bentracimab. “Blood products” include whole blood but not PRBCs.

In some embodiments, achievement of effective hemostasis following administration of an antibody or antigen-binding fragment thereof that binds to ticagrelor or a metabolite or derivative thereof to a patient experiencing major bleeding was determined using the criteria set out in Table 2. In some embodiments, the patient has excellent hemostasis following administration of the antibody or antigen-binding fragment thereof, as determined by the following criteria for visible bleeding:
Cessation of visible bleeding ≤1 hour after end of infusion, and
No additional coagulation intervention required.

In some embodiments, the patient has excellent hemostasis following administration of the antibody or antigen-binding fragment thereof, as determined by the following criteria for non-visible bleeding:
Muscular/skeletal: pain relief or no increase in swelling or unequivocal improvement in objective signs of bleeding ≤1 hour after the end of infusion; and the condition has not deteriorated during the 12-hour period after the end of infusion
Intracerebral hematoma (ICH): ≤20% increase in hematoma volume compared to baseline on a repeat CT or MRI scan performed at both the 1 and 12 hour post infusion time points
Subarachnoid bleed: ≤20% increase in maximum thickness using the most dense area on the follow-up vs baseline at both the 1 and 12 hour post infusion time points
Subdural hematoma: ≤20% increase in maximum thickness at both the 1 and 12 hour post infusion assessments compared to baseline
Pericardial: No increase in the size of pericardial effusion on repeat echocardiogram done within 12 hours of the end of infusion
Intraspinal: No increase in the size of pericardial effusion on repeat CT or MRI done within 12 hours of the end of infusion
Non-visible bleeding not described above (e.g., gastrointestinal, urinary): ≤10% decrease in both corrected hemoglobin/hematocrit at 24 hours compared to baseline

In some embodiments, the patient has good (effective) hemostasis following administration of the antibody or antigen-binding fragment thereof, as determined by the following criteria for visible bleeding:
Cessation of bleeding between >1 and ≤4 hours after end of infusion, and
No additional coagulation intervention required.

In some embodiments, the patient has good (effective) hemostasis following administration of the antibody or antigen-binding fragment thereof, as determined by the following criteria for non-visible bleeding:
Muscular/skeletal: pain relief or no increase in swelling or unequivocal improvement in objective signs of bleeding >1 and ≤4 hours after end of infusion; and the condition has not deteriorated during the 12-hour period after the end of infusion
ICH: >20% but ≤35% increase in hematoma volume compared to baseline on a repeat CT or MRI scan at +12-hour time point after the end of infusion
Subarachnoid bleed: >20% but <35% increase in maximum thickness using the most dense area on the follow-up at +12h after end of infusion vs baseline
Subdural hematoma: >20% but <35% increase in maximum thickness at +12h after end of infusion compared to baseline
Pericardial: <10% increase in the size of pericardial effusion on repeat echocardiogram done within 12 hours of the end of infusion
Intraspinal: <10% increase in the size of pericardial effusion on repeat CT or MRI done within 12 hours of the end of infusion
Non-visible bleeding not described above (e.g., gastrointestinal, urinary): >10% to ≤20% decrease in both corrected hemoglobin/hematocrit at 24 hours compared to baseline

In some embodiments, the patient has poor or no (not effective) hemostasis following administration of the antibody or antigen-binding fragment thereof, as determined by the following criteria for visible bleeding:
Cessation of bleeding >4 hours after end of the infusion; and/or
Additional coagulation intervention required (e.g., plasma, whole blood, or coagulation factor products.

In some embodiments, the patient has good (effective) hemostasis following administration of the antibody or antigen-binding fragment thereof, as determined by the following criteria for non-visible bleeding:
Muscular/skeletal: No improvement by 4 hours after end of infusion and/or condition has deteriorated during the 12-hour period
ICH: >35% increase in hematoma volume on a CT or MRI compared to baseline on a repeat CT or MRI scan at +12-hour time point after the end of infusion
Subarachnoid bleed: >35% increase in maximum thickness using the most dense area on the follow-up at +12h after end of infusion vs baseline
Subdural hematoma: >35% increase in maximum thickness at +12h after end of infusion compared to baseline
Pericardial: 10% or more increase in the size of pericardial effusion on repeat echocardiogram done within 12 hours of the end of infusion
Intraspinal: 10% or more increase in the size of pericardial effusion on repeat CT or MRI done within 12 hours of the end of infusion
Non-visible bleeding not described above (e.g., gastrointestinal, urinary): >20% decrease in both corrected hemoglobin/hematocrit.

In some embodiments, the patient experiencing major bleeding is administered the antibody or antigen-binding fragment thereof during surgery. In some embodiments, the antibody or antigen-binding fragment thereof is bentracimab.

TABLE 3
Rating System for Effective Hemostasis in Surgical Patients

| Hemostasis | Procedure-Related GUSTO Bleeding |
|---|---|
| Effective | No bleeding; or |
| | GUSTO mild bleeding; or |
| | GUSTO moderate bleeding |
| Not effective | GUSTO severe bleeding |

Additional Factors to be Considered During Adjudication

Any additional intraoperative or postoperative hemostasis-
related data will be taken into account for the hemostasis
assessment, i.e., estimated blood loss, intraoperative
transfusions, diagnostic imaging, etc.
Any bleeding events occurring >24 hours after initiation of
bentracimab that are procedure-related will be taken into
account for the hemostasis assessment
GUSTO mild bleeding: does not meet criteria for either
moderate or severe bleeding
Footnotes:
The operative hemostasis scale is based on a modified GUSTO bleeding scale (below) (GUSTO. (1993). N Engl J Med, 673-82):
a. GUSTO mild bleeding: does not meet criteria for either moderate or severe bleeding
b. GUSTO moderate bleeding requires blood transfusion but does not result in hemodynamic compromise
c. GUSTO severe bleeding: fatal bleeding, intracranial hemorrhage, or bleeding that causes hemodynamic compromise and requires pharmacologic or surgical intervention

In some embodiments, achievement of effective hemostasis following administration of an antibody or antigen-binding fragment thereof that binds to ticagrelor or a metabolite or derivative thereof to a patient who is in need of surgery or an invasive procedure was determined using the criteria set out in Table 3. In some embodiments, achievement of effective hemostasis following administration of the antibody or antigen-binding fragment thereof to a patient requiring surgery was determined using prespecified criteria for effective hemostasis derived from the GUSTO (Global Utilization of Streptokinase and Tpa for Occluded Arteries) clinical bleeding scale. See, GUSTO. (1993). N Engl J Med, 673-82. In some embodiments, the patient has excellent hemostasis following administration of bentracimab, as determined by the following criteria:
No bleeding,
GUSTO mild bleeding,
or GUSTO moderate bleeding.

In some embodiments, a criterion for non-effective hemostasis following administration of the antibody or antigen-binding fragment thereof to the patient in need of surgery or an invasive procedure is the patient having GUSTO severe bleeding. In some embodiments, the antibody or antigen-binding fragment thereof is bentracimab.

In some embodiments, administration of the antibody or antigen-binding fragment thereof that binds to ticagrelor or a metabolite or derivative thereof to a population of patients results in a treatment-emergent adverse event related to the administration of the antibody or antigen-binding fragment thereof in less than 2% of patients within the population of patients. In some embodiments, administration of the antibody or antigen-binding fragment thereof to a population of patients results in a treatment-emergent adverse event related to the administration of the antibody or antigen-binding fragment thereof in less than 2% of patients within the population of patients, wherein the patient received the antibody or antigen-binding fragment during surgery, in a perioperative setting, or during an invasive procedure. In some embodiments, administration of the antibody or antigen-binding fragment thereof to a population of patients results in a treatment-emergent adverse event related to the administration of the antibody or antigen-binding fragment thereof in about 2% to about 3% of patients within the population of patients. In some embodiments, administration of the antibody or antigen-binding fragment thereof to a population of patients results in a treatment-emergent adverse event related to the administration of the antibody or antigen-binding fragment thereof in about 0% to about 3% of patients within the population of patients. In some embodiments, administration of the antibody or antigen-binding fragment thereof to a population of patients results in a treatment-emergent adverse event related to the administration of the antibody or antigen-binding fragment thereof in about 0% to about 2.5% of patients within the population of patients. In some embodiments, administration of the antibody or antigen-binding fragment thereof to a population of patients results in a treatment-emergent adverse event related to the administration of the antibody or antigen-binding fragment thereof in about 1% to about 2.5% of patients within the population of patients. In some embodiments, administration of the antibody or antigen-binding fragment thereof to a population of patients results in a treatment-emergent adverse event related to the administration of the antibody or antigen-binding fragment thereof in less than 3% of patients within the population of patients. In some embodiments, administration of the antibody or antigen-binding fragment thereof to a population of patients results in a treatment-emergent adverse event related to the administration of the antibody or antigen-binding fragment thereof in less than 3% of patients within the population of patients, wherein the patient has uncontrolled major or life-threatening bleeding. In some embodiments, the antibody or antigen-binding fragment thereof is administered to the patient during surgery, in a perioperative setting, or during an invasive procedure. In some embodiments, the antibody or antigen-binding fragment thereof is administered to the patient, wherein the patient is elderly, has underlying cardiac or pulmonary or metabolic disease, and/or has limited cardiac reserve. In some embodiments, administration of the antibody or antigen-binding fragment thereof results in improved percent inhibition of PRU in the patient. In some embodiments, the antibody or antigen-binding fragment thereof is administered in the following schedule: 6 g infused over 10 minutes, followed by 6 g over 4 hours, followed by 6 g over 12 hours.

In some embodiments, risk for one or more adverse clinical outcomes are lowered for a first patient who is administered the antibody or antigen-binding fragment thereof that binds to ticagrelor after receiving ticagrelor compared to a second patient who is not administered the antibody or antigen-binding fragment thereof after receiving ticagrelor, as described in Example 2. In some embodiments, the adverse clinical outcome is severe bleeding. In some embodiments, the adverse clinical outcome is mortality. In some embodiments, the antibody or antigen-binding fragment thereof that binds to ticagrelor is bentracimab.

In some embodiments, a first patient who is administered the antibody or antigen-binding fragment thereof after receiving ticagrelor has a lower risk of severe bleeding compared to a second patient who is not administered the antibody or antigen-binding fragment thereof after receiving ticagrelor, wherein the first patient and the second patient both require urgent surgery. In some embodiments, the risk of severe bleeding of the first patient who is administered the antibody or antigen-binding fragment thereof after receiving ticagrelor is about 17.7 to about 29.0 lower than that of the second patient who is not administered the antibody or antigen-binding fragment thereof after receiving ticagrelor. In some embodiments, the risk of severe bleeding of the first patient who is administered the antibody or antigen-binding fragment thereof after receiving ticagrelor is about 23.4 lower than that of the second patient who is not administered the antibody or antigen-binding fragment thereof. In some embodiments, the urgent surgery is CABG, and wherein the risk of severe bleeding of the first patient who is administered the antibody or antigen-binding fragment thereof after receiving ticagrelor is about 14.8 to about 31.6 lower than that of the second patient who is not administered the antibody or antigen-binding fragment thereof after receiving ticagrelor. In some embodiments, the risk of severe bleeding of the first patient who is administered the antibody or antigen-binding fragment thereof after receiving ticagrelor is about 22.5 lower than that of the second patient who is not administered the antibody or antigen-binding fragment thereof after receiving ticagrelor. In some embodiments, a first population of patients requiring urgent surgery and who are administered the antibody or antigen-binding fragment thereof after receiving ticagrelor has a lower risk of severe bleeding compared to a second population of patients requiring urgent surgery and who are not administered the antibody or antigen-binding fragment thereof after receiving ticagrelor. In some embodiments, the risk of severe bleeding of the first population of patients who are administered the antibody or antigen-binding fragment thereof after receiving ticagrelor is about 17.7 to about 29.0 lower than that of the second population of patients who is not administered the antibody or antigen-binding fragment thereof after receiving ticagrelor. In some embodiments, the urgent surgery is CABG, and wherein the risk of severe bleeding of the first population of patients who are administered the antibody or antigen-binding fragment thereof after receiving ticagrelor is about 14.8 to about 31.6 lower than that of the second population of patients who are not administered the antibody or antigen-binding fragment thereof after receiving ticagrelor. In some embodiments, the risk of severe bleeding of the first population of patients who are administered the antibody or antigen-binding fragment thereof after receiving ticagrelor is about 22.5 lower than that of the second population of patients who are not administered the antibody or antigen-binding fragment thereof after receiving ticagrelor. In some embodiments, the administered the antibody or antigen-binding fragment thereof that binds to ticagrelor is bentracimab. In some embodiments, a patient has severe bleeding if effective hemostasis is not achieved within one day after start of surgery. In some embodiments, the severe bleeding is life-threatening. In some embodiments, the severe bleeding is measured on the GUSTO scale, i.e., GUSTO severe bleeding. In some embodiments, the risk is a relative risk.

In some embodiments, a first patient who is administered the antibody or antigen-binding fragment thereof that binds to ticagrelor after receiving ticagrelor has a lower risk of mortality compared to a second patient who is not administered the antibody or antigen-binding fragment thereof after receiving ticagrelor, wherein the first patient or second patient both have major bleeding. In some embodiments, the major bleeding is procedure-related bleeding, non-procedure-related or spontaneous major bleeding, non-procedure-related or spontaneous intracerebral hematoma (ICH), or non-procedure-related or spontaneous non-ICH bleeding. In some embodiments, the major bleeding is procedure-related bleeding. In some embodiments, the major bleeding is spontaneous ICH. In some embodiments, the major bleeding is non-procedure-related or spontaneous major bleeding. In some embodiments, the major bleeding is non-procedure-related or spontaneous non-ICH bleeding. In some embodiments, the procedure-related major bleeding is related to surgery or invasive procedure. In some embodiments, the surgery is cardiac surgery, gastrointestinal surgery, vascular surgery, thoracic surgery, neurosurgery, gastrointestinal surgery, urogenital surgery, or major orthopedic surgery. In some embodiments, the surgery is coronary artery bypass graft (CABG), cardiac surgery, gastrointestinal surgery, coronary artery procedure, repair of aortic dissection, and/or cardiac valve replacement. In some embodiments, the surgery is CABG. In some embodiments, the surgery is mediastinal surgery. In some embodiments, the surgery or invasive procedure is invasive cardiothoracic procedures, vascular surgery, abdominal surgery, and/or surgery for traumatic injuries. In some embodiments, the major bleeding is spontaneous, or unrelated to surgery or invasive procedure. In some embodiments, the spontaneous non-ICH bleeding is intracranial hemorrhage, gastrointestinal bleeding, intra-articular bleeding, pericardial bleeding, or intramuscular bleeding with compartment syndrome. In some embodiments, intracranial hemorrhage is any bleeding inside the cranium. In some embodiments, intracranial hemorrhage is epidural hematoma, subdural hematoma, subarachnoid hemorrhage, or intraventricular hemorrhage. In some embodiments, the risk of mortality of the first patient who is administered the antibody or antigen-binding fragment after receiving ticagrelor is about 1.0 to about 16.4 lower than that of the second patient who is not administered the antibody or antigen-binding fragment thereof after receiving ticagrelor. In some embodiments, the risk of mortality of the first patient who is administered the antibody or antigen-binding fragment after receiving ticagrelor is about 7.7 lower than that of the second patient who is not administered the antibody or antigen binding fragment thereof after receiving ticagrelor. In some embodiments, a first population of patients with major bleeding and who are administered the antibody or antigen-binding fragment thereof after receiving ticagrelor has a lower risk of mortality compared to a second population of patients with major bleeding and who are not administered the antibody or antigen-binding fragment thereof after receiving ticagrelor. In some embodiments, the risk of mortality of the first population of patients who is administered the antibody or antigen-binding fragment thereof after receiving ticagrelor is about 1.0 to about 16.4 lower than that of the second population of patients who is not administered the antibody or antigen-binding fragment thereof after receiving ticagrelor. In some embodiments, the antibody or antigen-binding fragment thereof is administered to the first patient during surgery or during an invasive procedure. In some embodiments, the antibody or antigen-binding fragment thereof is administered to the first population of patients during surgery or during an invasive procedure. In some embodiments, the administered the antibody or antigen-binding fragment thereof that binds to ticagrelor is bentracimab. In some embodiments, risk of mortality is calculated using weighted logistic regression with sIPTW propensity score weights to balance patient characteristics between the treatment groups and adjusting for type of bleed (procedure-related, spontaneous ICH, and spontaneous non-ICH) and days off ticagrelor (0, 1 or 2). In some embodiments, the mortality is due to any cause within 38 days after start of major bleeding. In some embodiments, the risk is a relative risk.

In some embodiments, the first patient who is administered the antibody or antigen-binding fragment thereof that binds to ticagrelor after receiving ticagrelor has a lower risk of mortality compared to a second patient who is not administered the antibody or antigen-binding fragment thereof after receiving ticagrelor, wherein the first patient and second patient both require urgent surgery. In some embodiments, the risk of mortality of the first patient who is administered the antibody or antigen-binding fragment thereof after receiving ticagrelor is at least about 1.3 to about 11.4 lower than that of the second patient who is not administered the antibody or antigen-binding fragment thereof after receiving ticagrelor. In some embodiments, the risk of mortality of the first patient who is administered the antibody or antigen-binding fragment thereof after receiving ticagrelor is about 6.6 lower than that of the second patient who is not administered the antibody or antigen-binding fragment thereof after receiving ticagrelor. In some embodiments, a first population of patients requiring urgent surgery and who are administered the antibody or antigen-binding fragment thereof after receiving ticagrelor has a lower risk of mortality compared to a second population of patients requiring urgent surgery and who are not administered the antibody or antigen-binding fragment thereof after receiving ticagrelor. In some embodiments, the risk of mortality of the first population of patients who are administered the antibody or antigen-binding fragment thereof after receiving ticagrelor is at least about 1.3 to about 11.4 lower than that of the second population of patients who are not administered the antibody or antigen-binding fragment thereof after receiving ticagrelor. In some embodiments, the risk of mortality of the first population of patients who are administered the antibody or antigen-binding fragment thereof after receiving ticagrelor is about 6.6 lower than that of the second population of patients who are not administered the antibody or antigen-binding fragment thereof after receiving ticagrelor. In some embodiments, the administered the antibody or antigen-binding fragment thereof that binds to ticagrelor is bentracimab. In some embodiments, the surgery is associated with a risk of significant bleeding. In some embodiments, the surgery is cardiac surgery, gastrointestinal surgery, vascular surgery, thoracic surgery, neurosurgery, gastrointestinal surgery, urogenital surgery, or major orthopedic surgery. In some embodiments, the surgery is coronary artery bypass graft (CABG), cardiac surgery, gastrointestinal surgery, coronary artery procedure, repair of aortic dissection, and/or cardiac valve replacement. In some embodiments, the surgery is CABG. In some embodiments, the surgery is mediastinal surgery. In some embodiments, the surgery or invasive procedure is invasive cardiothoracic procedures, vascular surgery, abdominal surgery, and/or surgery for traumatic injuries. In some embodiments, risk of mortality is calculated using weighted logistic regression with sIPTW propensity score weights to balance patient characteristics between the treatment groups and adjusting for type of bleed (procedure-related, spontaneous ICH, and spontaneous non-ICH) and days off ticagrelor (0, 1 or 2). In some embodiments, the mortality is due to any cause within 38 days after start of urgent surgery. In some embodiments, the risk is a relative risk.

In some embodiments, a first patient who is administered the antibody or antigen-binding fragment thereof that binds to ticagrelor after receiving ticagrelor has a lower risk of mortality compared to a second patient who is not administered the antibody or antigen-binding fragment thereof after receiving ticagrelor, wherein the first patient and second patient require urgent surgery or has major bleeding. In some embodiments, the risk of mortality of the first patient who is administered the antibody or antigen-binding fragment thereof after receiving ticagrelor is at least about 0.5 to about 9.5 lower than that of the second patient who is not administered the antibody or antigen-binding fragment thereof after receiving ticagrelor. In some embodiments, the risk of mortality of the first patient who is administered the antibody or antigen-binding fragment thereof after receiving ticagrelor is about 5.4 lower than that of the second patient who is not administered the antibody or antigen-binding fragment thereof after receiving ticagrelor. In some embodiments, a first population of patients who are administered the antibody or antigen-binding fragment thereof after receiving ticagrelor has a lower risk of mortality compared to a second population of patients who are not administered the antibody or antigen-binding fragment thereof after receiving ticagrelor, wherein the first population of patients and the second population of patients require urgent surgery or have major bleeding. In some embodiments, the risk of mortality of the first population of patients who are administered the antibody or antigen-binding fragment thereof after receiving ticagrelor is at least about 0.5% to about 9.5 lower than that of the second population of patients who are not administered the antibody or antigen-binding fragment thereof after receiving ticagrelor. In some embodiments, the risk of mortality of the first population of patients who are administered the antibody or antigen-binding fragment thereof after receiving ticagrelor is about 5.4 lower than that of the second population of patients who are not administered the antibody or antigen-binding fragment thereof after receiving ticagrelor. In some embodiments, the administered the antibody or antigen-binding fragment thereof that binds to ticagrelor is bentracimab. In some embodiments, the surgery is associated with a risk of significant bleeding. In some embodiments, the surgery is cardiac surgery, gastrointestinal surgery, vascular surgery, thoracic surgery, neurosurgery, gastrointestinal surgery, urogenital surgery, or major orthopedic surgery. In some embodiments, the surgery is coronary artery bypass graft (CABG), cardiac surgery, gastrointestinal surgery, coronary artery procedure, repair of aortic dissection, and/or cardiac valve replacement. In some embodiments, the surgery is CABG. In some embodiments, the surgery is mediastinal surgery. In some embodiments, the surgery or invasive procedure is invasive cardiothoracic procedures, vascular surgery, abdominal surgery, and/or surgery for traumatic injuries. In some embodiments, risk of mortality is calculated using weighted logistic regression with sIPTW propensity score weights to balance patient characteristics between the treatment groups and adjusting for type of bleed (procedure-related, spontaneous ICH, and spontaneous non-ICH) and days off ticagrelor (0, 1 or 2). In some embodiments, the mortality is due to any cause within 38 days after start of major bleeding or within 38 days after start of urgent surgery. In some embodiments, the risk is a relative risk.

In some embodiments, administering the antibody or antigen-binding fragment thereof that binds to ticagrelor or a metabolite or derivative thereof results in reduced risk of a first patient (or population of patients) receiving a first transfusion within 120 hours after receiving the antibody or antigen-binding fragment thereof. In some embodiments, the risk of a first transfusion is reduced compared to the risk of receiving a transfusion within 120 hours of an equivalent start a second patient (or population of patients) who did not receive the antibody or antigen-binding fragment thereof, wherein the first patient (or population of patients) and second patient (or population of patients) both have major bleeding. For the second population of patient who did not receive the antibody or antigen-binding fragment thereof, the equivalent start is defined as starting from the average amount of time after the major bleeding event that the first population of patients received the antibody or antigen-binding fragment thereof. For example, if the first population of patients received the antibody or antigen-binding fragment thereof 9 hours after start of major bleeding, the equivalent start for the 120 hour period for the second population of patients is 9 hours after start of major bleeding. In some embodiments, the risk ratio comparing the risk of receiving a first transfusion in the first patient or first population of patients who is administered the antibody or antigen-binding fragment thereof after receiving ticagrelor to the second patient or second population of patients who is not administered the antibody or antigen-binding fragment thereof after receiving ticagrelor is about 0.105 to about 0.413. In some embodiments, the risk ratio comparing the risk of receiving a first transfusion in the first patient or first population of patients who is administered the antibody or antigen-binding fragment thereof after receiving ticagrelor to the second patient or second population of patients who is not administered the antibody or antigen-binding fragment thereof after receiving ticagrelor is about 0.259. In some embodiments, the major bleeding is procedure-related major bleeding. In some embodiments, the major bleeding is procedure-related major bleeding, and the risk ratio comparing the risk of receiving a first transfusion in the first patient or first population of patients who is administered the antibody or antigen-binding fragment thereof after receiving ticagrelor to the second patient or second population of patients who is not administered the antibody or antigen-binding fragment thereof after receiving ticagrelor is about 0.286 to about 0.800. In some embodiments, the major bleeding is procedure-related major bleeding, and the risk ratio comparing the risk of receiving a first transfusion in the first patient or first population of patients who is administered the antibody or antigen-binding fragment thereof after receiving ticagrelor to the second patient or second population of patients who is not administered the antibody or antigen-binding fragment thereof after receiving ticagrelor is about 0.543. In some embodiments, the major bleeding is spontaneous major bleeding. In some embodiments, the major bleeding is spontaneous major bleeding, and the risk ratio comparing the risk of receiving a first transfusion in the first patient or first population of patients who is administered the antibody or antigen-binding fragment thereof after receiving ticagrelor to the second patient or second population of patients who is not administered the antibody or antigen-binding fragment thereof after receiving ticagrelor is about −0.045 to about 0.190. In some embodiments, the major bleeding is spontaneous major bleeding, and the risk ratio comparing the risk of receiving a first transfusion in the first patient or first population of patients who is administered the antibody or antigen-binding fragment thereof after receiving ticagrelor to the second patient or second population of patients who is not administered the antibody or antigen-binding fragment thereof after receiving ticagrelor is about 0.072. In some embodiments, the spontaneous major bleeding is non-intracranial hemorrhage (non-ICH) spontaneous major bleeding or ICH spontaneous major bleeding. In some embodiments, the non-ICH spontaneous major bleeding is gastrointestinal bleeding. In some embodiments, the antibody or antigen-binding fragment thereof is bentracimab. In some embodiments, the antibody or antigen-binding fragment thereof is administered to the first patient or first population of patients during surgery or during an invasive procedure.

In some embodiments, administering the antibody or antigen-binding fragment thereof that binds to ticagrelor or a metabolite or derivative thereof results in increased time between receiving the antibody or antigen-binding fragment thereof and receiving a first transfusion for a first patient (or population of patients) receiving a first transfusion within 120 hours after receiving the antibody or antigen-binding fragment thereof. In some embodiments, the time between receiving bentracimab and receiving a first transfusion is increased compared to the time between an equivalent start and receiving a first transfusion in a second patient or second population of patients who is not administered the antibody or antigen-binding fragment thereof after receiving ticagrelor, wherein both the first patient (or population of patients) and second patient (or population of patients) have major bleeding. For the second population of patient who did not receive the antibody or antigen-binding fragment thereof, the equivalent start is defined as starting from the average amount of time after the major bleeding event that the first population of patients received the antibody or antigen-binding fragment thereof. For example, if the first population of patients received the antibody or antigen-binding fragment thereof 9 hours after start of major bleeding, the equivalent start for the 120 hour period for the second population of patients is 9 hours after start of major bleeding. In some aspects, provided herein are methods of reversing ticagrelor-enhanced bleeding in a patient comprising administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient.

In some aspects, provided herein are methods of reversing ticagrelor-enhanced bleeding in a patient (or population of patients) comprising: i) administering to the patient (or population of patients) an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof, then ii) administering a transfusion to the patient (or population of patients) at least 10 hours after (i) administering the antibody or antigen-binding fragment thereof. In some embodiments, the patient has major bleeding. In some embodiments, the major bleeding is procedure-related major bleeding. In some embodiments, the major bleeding is spontaneous major bleeding. In some embodiments, the spontaneous major bleeding is non-intracranial hemorrhage (non-ICH) spontaneous major bleeding or ICH spontaneous major bleeding. In some embodiments, the non-ICH spontaneous major bleeding is gastrointestinal bleeding. In some embodiments, the antibody or antigen-binding fragment thereof is bentracimab. In some embodiments, the antibody or antigen-binding fragment thereof is administered to the first patient or first population of patients during surgery or during an invasive procedure.

In some embodiments, administering the antibody or antigen-binding fragment thereof that binds to ticagrelor or a metabolite or derivative thereof results in a reduced number of transfusions within 120 hours after start of major bleeding received by a first patient (or population of patients) receiving the antibody or antigen-binding fragment thereof. In some embodiments, the number of transfusions within 120 hours after start of major bleeding is reduced compared to number of transfusions within 120 hours after start of major bleeding received by a second patient or second population of patients who is not administered the antibody or antigen-binding fragment thereof, wherein both the first patient (or population of patients) and second patient (or population of patients) have major bleeding. In some embodiments, the major bleeding is spontaneous major bleeding. In some embodiments, the spontaneous major bleeding is non-intracranial hemorrhage (non-ICH) spontaneous major bleeding or ICH spontaneous major bleeding. In some embodiments, the non-ICH spontaneous major bleeding is gastrointestinal bleeding. In some embodiments, the antibody or antigen-binding fragment thereof is bentracimab. In some embodiments, the antibody or antigen-binding fragment thereof is administered to the first patient or first population of patients during surgery or during an invasive procedure.

In some aspects, provided herein are methods of reversing ticagrelor-enhanced bleeding in a patient (or population of patients) comprising: i) administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to a population of patients, wherein the population of patients have major bleeding, then ii) administering no more than a mean of 1 transfusion to the population of patients within 120 hours after start of major bleeding in the population of patients who had major bleeding. In some embodiments, the major bleeding is spontaneous major bleeding. In some embodiments, the spontaneous major bleeding is non-intracranial hemorrhage (non-ICH) spontaneous major bleeding or ICH spontaneous major bleeding. In some embodiments, the non-ICH spontaneous major bleeding is gastrointestinal bleeding. In some embodiments, the antibody or antigen-binding fragment thereof is bentracimab. In some embodiments, the antibody or antigen-binding fragment thereof is administered to the first patient or first population of patients during surgery or during an invasive procedure.

In some embodiments, administering the antibody or antigen-binding fragment thereof that binds to ticagrelor or a metabolite or derivative thereof results in reduced risk of requiring surgery to treat bleeding within 48 hours after the start of major bleeding in a first patient (or population of patients) receiving the antibody or antigen-binding fragment thereof. In some embodiments, the risk of requiring surgery to treat bleeding within 48 hours after the start of major bleeding is reduced compared to risk of requiring surgery to treat bleeding within 48 hours after the start of major bleeding in a second patient or second population of patients who is not administered the antibody or antigen-binding fragment thereof, wherein both the first patient (or population of patients) and second patient (or population of patients) have major bleeding. In some embodiments, the risk ratio comparing the risk of requiring surgery to treat bleeding within 48 hours after the start of major bleeding in a first patient or first population of patients who is administered the antibody or antigen-binding fragment thereof after receiving ticagrelor compared that of the second patient or second population of patients who is not administered the antibody or antigen-binding fragment thereof after receiving ticagrelor is about −0.051 to about 0.783. In some embodiments, the risk ratio comparing the risk of requiring surgery to treat bleeding within 48 hours after the start of major bleeding in a first patient or first population of patients who is administered the antibody or antigen-binding fragment thereof after receiving ticagrelor compared that of the second patient or second population of patients who is not administered the antibody or antigen-binding fragment thereof after receiving ticagrelor is about 0.366. In some embodiments, the major bleeding is spontaneous major bleeding. In some embodiments, the spontaneous major bleeding is non-intracranial hemorrhage (non-ICH) spontaneous major bleeding or ICH spontaneous major bleeding. In some embodiments, the non-ICH spontaneous major bleeding is gastrointestinal bleeding. In some embodiments, the antibody or antigen-binding fragment thereof is bentracimab. In some embodiments, the antibody or antigen-binding fragment thereof is administered to the first patient or first population of patients during surgery or during an invasive procedure.

In some embodiments, administration of the antibody or antigen-binding fragment thereof that binds to ticagrelor or a metabolite or derivative thereof to a population of patients results in a treatment-emergent adverse event related to the administration of the antibody or antigen-binding fragment thereof in less than about 0.5% of patients within the population of patients, wherein the treatment-emergent adverse event requires discontinuation of administration of the antibody or antigen-binding fragment thereof. In some embodiments, administration of the antibody or antigen-binding fragment thereof to a population of patients results in a treatment-emergent adverse event related to the administration of the antibody or antigen-binding fragment thereof resulting in death in less than about 5% of patients within the population of patients. In some embodiments, the population of patients has major bleeding. In some embodiments, the major bleeding is spontaneous major bleeding. In some embodiments, the spontaneous major bleeding is non-intracranial hemorrhage (non-ICH) spontaneous major bleeding or ICH spontaneous major bleeding. In some embodiments, the non-ICH spontaneous major bleeding is gastrointestinal bleeding. In some embodiments, the antibody or antigen-binding fragment thereof is bentracimab. In some embodiments, the antibody or antigen-binding fragment thereof is administered to the first patient or first population of patients during surgery or during an invasive procedure.

### EXEMPLARY EMBODIMENTS

Various embodiments provided herein are included in the following non-limiting list of embodiments.

Embodiment 1. A method of reversing ticagrelor-enhanced bleeding in a patient comprising administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient during surgery or during an invasive procedure, wherein the antibody or antigen-binding fragment thereof comprises a heavy chain complementarity determining region (CDRH)1, a CDRH2, and a CDRH3 of a variable heavy chain region (VH) comprising the amino acid sequence set forth in SEQ ID NO:01 and a light chain complementarity determining region (CDRL)1, CDRL2, and a CDRL3 of a variable light chain region (VL) comprising the amino acid sequence set forth in SEQ ID NO:02.

Embodiment 2. The method of embodiment 1, wherein the antibody or antigen-binding fragment thereof is administered during surgery.

Embodiment 3. The method of embodiment 2, wherein the surgery is associated with a risk of significant bleeding.

Embodiment 4. The method of embodiment 2 or 3, wherein the surgery is cardiac surgery, gastrointestinal surgery, vascular surgery, thoracic surgery, neurosurgery, urogenital surgery, or major orthopedic surgery.

Embodiment 5. The method of any one of embodiments 2-4, wherein the surgery has an adverse procedural outcome if hemostasis is impaired.

Embodiment 6. The method of embodiment 5, wherein the surgery is coronary artery bypass graft (CABG), cardiac surgery, gastrointestinal surgery, coronary artery procedure, repair of aortic dissection, and/or cardiac valve replacement.

Embodiment 7. The method of embodiment 6, wherein the surgery is CABG.

Embodiment 8. The method of any one of embodiments 1-7, wherein administering the antibody or antigen-binding fragment thereof to the patient results in a minimum percent inhibition of P2Y₁₂ reaction units (PRU) over 4 hours after starting administration of between about −78 and about −56 in the patient.

Embodiment 9. The method of any one of embodiments 1-8, wherein administering the antibody or antigen-binding fragment thereof to a population of patients results in a mean minimum percent inhibition of PRU over 4 hours after starting administration of between about −78 and about −56 in the population of patients.

Embodiment 10. The method of any one of embodiments 2-9, wherein the patient does not receive treatment with the antibody or antigen-binding fragment thereof prior to start of surgery.

Embodiment 11. The method of embodiment 1, wherein the antibody or antigen-binding fragment thereof is administered in a perioperative setting.

Embodiment 12. The method of embodiment 1, wherein the antibody or antigen-binding fragment thereof is administered during an invasive procedure.

Embodiment 13. The method of embodiment 12, wherein the invasive procedure is percutaneous cardiac intervention or another cardiac or vascular procedure.

Embodiment 14. The method of embodiment 12, wherein the invasive procedure is a cardiac valve replacement.

Embodiment 15. A The method of any of embodiments 1-14, wherein about 18 g, about 24 g, or about 36 g of the antibody or antigen-binding fragment thereof is administered.

Embodiment 16. The method of any of embodiments 1-15, wherein the antibody or antigen-binding fragment thereof is administered over about 10 min to about 36 hours.

Embodiment 17. The method of any of embodiments 1-16, wherein the antibody or antigen-binding fragment thereof is administered according to a schedule comprising about 6 g over about 10 minutes, followed by about 6 g over about 4 hours, and followed by about 6 g over about 12 hours.

Embodiment 18. The method of any of embodiments 1-16, wherein the antibody or antigen-binding fragment thereof is administered according to a schedule comprising about 6 g over about 10 minutes, followed by about 6 g over about 4 hours, followed by about 6 g over about 12 hours, and followed by about 6 g over about 12 hours.

Embodiment 19. The method of any of embodiments 1-16, wherein the antibody or antigen-binding fragment thereof is administered according to a schedule comprising about 12 g over about 10 minutes, followed by about 12 g over about 6 hours, and followed by about 12 g over about 12 hours.

Embodiment 20. The method of embodiment 18, wherein the has been treated with a moderate or strong CYP3A inhibitor.

Embodiment 21. A method of reversing ticagrelor-enhanced bleeding in a patient comprising administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient, wherein the antibody or antigen-binding fragment thereof comprises a CDRH1, a CDRH2, and a CDRH3 of a VH comprising the amino acid sequence set forth in SEQ ID NO:01 and a CDRL1, CDRL2, and a CDRL3 of a VL comprising the amino acid sequence set forth in SEQ ID NO:02, wherein the patient has metabolic disease; optionally, the patient is elderly and/or has underlying cardiac or pulmonary disease and/or has limited cardiac reserve. Embodiment 22. A method of reversing ticagrelor-enhanced bleeding in a patient comprising administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient, wherein the antibody or antigen-binding fragment thereof comprises a CDRH1, a CDRH2, and a CDRH3 of a VH comprising the amino acid sequence set forth in SEQ ID NO:01 and a CDRL1, CDRL2, and a CDRL3 of a VL comprising the amino acid sequence set forth in SEQ ID NO:02,
wherein the patient has a minimum percent inhibition of P2Y₁₂ reaction units (PRU) over 4 hours after starting administration of the antibody or antigen-binding fragment thereof that is less than the percent inhibition of PRU before administration, thereby reversing ticagrelor-enhanced bleeding.

Embodiment 23. The method of embodiment 22, wherein percent inhibition of PRU is determined by calculating 100×[(180−PRU)/180].

Embodiment 24. A method of reversing ticagrelor-enhanced bleeding in a patient comprising:
i) administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient, wherein the antibody or antigen-binding fragment thereof comprises a CDRH1, a CDRH2, and a CDRH3 of a VH comprising the amino acid sequence set forth in SEQ ID NO:01 and a CDRL1, CDRL2, and a CDRL3 of a VL comprising the amino acid sequence set forth in SEQ ID NO:02, and
ii) improving percent inhibition of PRU in the patient.

Embodiment 25. A method of reversing ticagrelor-enhanced bleeding in a patient comprising administering an antibody or antigen-binding fragment thereof that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof to the patient, wherein the antibody or antigen-binding fragment thereof comprises a CDRH1, a CDRH2, and a CDRH3 of a VH comprising the amino acid sequence set forth in SEQ ID NO:01 and a CDRL1, CDRL2, and a CDRL3 of a VL comprising the amino acid sequence set forth in SEQ ID NO:02,
wherein administration of the antibody or antigen-binding fragment thereof to a population of patients results in a treatment-emergent adverse event related to the administration of the antibody or antigen-binding fragment thereof in less than 3% of patients within the population of patients.

Embodiment 26. The method of any one of embodiments 1-25, wherein the antibody or antigen-binding fragment thereof comprises CDR combinations comprising SEQ ID NO:03 (CDRH1), SEQ ID NO:04 (CDRH2), SEQ ID NO:05 (CDRH3), SEQ ID NO:06 (CDRL1), SEQ ID NO:07 (CDRL2), and SEQ ID NO:08 (CDRL3).

Embodiment 27. The method of any one of embodiments 1-26, wherein the VH has an amino acid sequence according to SEQ ID NO:01 and the VL has an amino acid sequence according to SEQ ID NO:02.

Embodiment 28. The method of any one of embodiments 1-27, wherein the antibody or antigen-binding fragment is an antibody.

Embodiment 29. The method of any one of embodiments 1-27, wherein the antibody or antigen-binding fragment is an antigen-binding fragment.

Embodiment 30. The method of embodiment 29, wherein the antigen-binding fragment is a Fab.

Embodiment 31. The method of any one of embodiments 1-30, wherein the antibody or antigen-binding fragment thereof is administered within about one day of the last dose of ticagrelor

Embodiment 32. The method of embodiment 31, wherein the antibody or antigen-binding fragment thereof is administered on the same day as the most recent dose of ticagrelor.

Embodiment 33. The method of any one of embodiments 1-32, wherein the patient was previously treated with a daily dose of between about 60 mg and about 360 mg of ticagrelor.

Embodiment 34. The method of any one of embodiments 1-33, wherein the patient was previously treated with ticagrelor for about 1 to 12 months.

Embodiment 35. The method of any one of embodiments 1-33, wherein the patient was previously treated with ticagrelor for about 0 to 1 month.

Embodiment 36. The method of any one of embodiments 1-33, wherein the patient was previously treated with ticagrelor for about 1 to 10 years.

Embodiment 37. The method of any one of embodiments 1-36, wherein, prior to administration of the antibody or antigen-binding fragment thereof, the patient has PRU of between about 180 to about 230.

Embodiment 38. The method of any one of embodiments 1-37, wherein PRU is not measured before administering the antibody or antigen-binding fragment thereof.

Embodiment 39. The method of any one of embodiments 1-38, wherein the patient is in need of gastrointestinal surgery, vascular surgery, thoracic surgery, or urogenital surgery.

Embodiment 40. The method of any one of embodiments 1-39, wherein the dose of acetylsalicylic acid is 0 mg to 325 mg.

Embodiment 41. The method of any one of embodiments 1-40, wherein the patient receives chronic treatment with acetylsalicylic acid.

Embodiment 42. The method of any one of embodiments 1-41, wherein administering the antibody or antigen-binding fragment thereof to the patient results in an increase in PRU in the patient.

Embodiment 43. The method of any one of embodiments 21-42, wherein administering the antibody or antigen-binding fragment thereof to the patient results in the patient having a minimum percent inhibition of PRU over 4 hours after starting administration of between about −76 and about −67, wherein the patient requires urgent surgery or has major bleeding.

Embodiment 44. The method of any one of embodiments 21-42, wherein administering the antibody or antigen-binding fragment thereof to a population of patients results in a mean minimum percent inhibition of PRU over 4 hours after starting administration of between about −76 and about −67 in the population of patients, wherein the population of patients requires urgent surgery or has major bleeding.

Embodiment 45. The method of any one of embodiments 21-42 or 44, wherein administering the antibody or antigen-binding fragment thereof to the population of patients with major bleeding or requiring urgent surgery results in a difference of about −129 to −115 between the minimum percent inhibition of PRU over 4 hours after starting administration and the percent inhibition of PRU before administration in the patient, wherein the minimum percent inhibition of P2Y₁₂ reaction units (PRU) over 4 hours after starting administration of the antibody or antigen-binding fragment thereof is less than the percent inhibition of PRU before administration.

Embodiment 46. The method of any one of embodiments 21-42 or 43, wherein administering the antibody or antigen-binding fragment thereof results in the patient with major bleeding or requiring urgent surgery achieving good or excellent homeostasis.

Embodiment 47. The method of embodiment 46, wherein administering the antibody or antigen-binding fragment thereof to the population of patients results in excellent or good hemostasis in about 95% of the population of patients.

Embodiment 48. The method of any one of embodiments 21-42, wherein administering the antibody or antigen-binding fragment thereof to the patient results in a minimum percent inhibition of P2Y₁₂ reaction units (PRU) over 4 hours after starting administration of between about −78 and about −56 in the patient, wherein the patient has uncontrolled major or life-threatening bleeding.

Embodiment 49. The method of any one of embodiments 21-42, wherein administering the antibody or antigen-binding fragment thereof to a population of patients with uncontrolled major or life-threatening bleeding results in a mean minimum percent inhibition of PRU over 4 hours after starting administration of between about −78 and about −56 in the population of patients.

Embodiment 50. The method of any one of embodiments 21-42 or 49, wherein administering the antibody or antigen-binding fragment thereof to the population of patients with uncontrolled major or life-threatening bleeding results in a difference of about −130 to −101 between the minimum percent inhibition of PRU over 4 hours after starting administration and the percent inhibition of PRU before administration in the patient, wherein the minimum percent inhibition of P2Y₁₂ reaction units (PRU) over 4 hours after starting administration of the antibody or antigen-binding fragment thereof is less than the percent inhibition of PRU before administration.

Embodiment 51. The method of any one of embodiments 21-42 or 48, wherein administering the antibody or antigen-binding fragment thereof to the patient with uncontrolled major or life-threatening bleeding results in the patient achieving excellent or good hemostasis.

Embodiment 52. The method of embodiment 51, wherein administering the antibody or antigen-binding fragment thereof to the population of patients with uncontrolled major or life-threatening bleeding results in excellent or good hemostasis in about 80% of the population of patients.

Embodiment 53. The method of any one of embodiments 21-42, wherein administering the antibody or antigen-binding fragment thereof to the patient results in a minimum percent inhibition of P2Y₁₂ reaction units (PRU) over 4 hours after starting administration of between about −78 and about −68 in the patient, wherein the patient requires surgery or invasive procedure.

Embodiment 54. The method of any one of embodiments 21-42, wherein administering the antibody or antigen-binding fragment thereof to a population of patients requiring surgery or invasive procedure results in a mean minimum percent inhibition of PRU over 4 hours after starting administration of between about −78 and about −68 in the population of patients.

Embodiment 55. The method of embodiment 21-42 or 54, wherein administering the antibody or antigen-binding fragment thereof to the population of patients requiring surgery or invasive procedure results in a difference of about −132 to −117 between the minimum percent inhibition of PRU over 4 hours after starting administration and the percent inhibition of PRU before administration in the patient, wherein the minimum percent inhibition of P2Y₁₂ reaction units (PRU) over 4 hours after starting administration of the antibody or antigen-binding fragment thereof is less than the percent inhibition of PRU before administration.

Embodiment 56. The method of any one of embodiments 21-42 or 53, wherein administering the antibody or antigen-binding fragment thereof to the patient requiring surgery or invasive procedure results in the patient achieving excellent or good hemostasis.

Embodiment 57. The method of embodiment 56, wherein administering the antibody or antigen-binding fragment thereof to a population of patients requiring surgery or invasive procedure results in excellent or good hemostasis in about 100% of the population of patients.

Embodiment 58. The method of any one of embodiments 1-57, further comprising resuming ticagrelor treatment after administering the antibody or antigen-binding fragment thereof, wherein treatment with ticagrelor is restarted 8 hours to 3 days after administering the antibody or antigen-binding fragment thereof.

Embodiment 59. The method of any one of embodiments 1-58, wherein the antibody or antigen-binding fragment thereof is administered to the patient intravenously.

Embodiment 60. The method of any one of embodiments 21-59, wherein a first patient who is administered the antibody or antigen-binding fragment thereof after receiving ticagrelor has a lower risk of severe bleeding compared to a second patient who is not administered the antibody or antigen-binding fragment thereof after receiving ticagrelor, wherein the first patient and the second patient both require urgent surgery.

Embodiment 61. The method of embodiment 60, wherein the risk of severe bleeding of the first patient who is administered the antibody or antigen-binding fragment thereof after receiving ticagrelor is about 17.7 to about 29.0 lower than that of the second patient who is not administered the antibody or antigen-binding fragment thereof after receiving ticagrelor; optionally wherein the risk of severe bleeding of the first patient who is administered the antibody or antigen-binding fragment thereof after receiving ticagrelor is about 23.4 lower than that of the second patient who is not administered the antibody or antigen-binding fragment thereof.

Embodiment 62. The method of embodiment 60, wherein the urgent surgery is CABG, and wherein the risk of severe bleeding of the first patient who is administered the antibody or antigen-binding fragment thereof after receiving ticagrelor is about 14.8 to about 31.6 lower than that of the second patient who is not administered the antibody or antigen-binding fragment thereof after receiving ticagrelor; optionally wherein the risk of severe bleeding of the first patient who is administered the antibody or antigen-binding fragment thereof after receiving ticagrelor is about 22.5 lower than that of the second patient who is not administered the antibody or antigen-binding fragment thereof after receiving ticagrelor.

Embodiment 63. The method of any one of embodiments 21-59, wherein a first population of patients requiring urgent surgery and who are administered the antibody or antigen-binding fragment thereof after receiving ticagrelor has a lower risk of severe bleeding compared to a second population of patients requiring urgent surgery and who are not administered the antibody or antigen-binding fragment thereof after receiving ticagrelor.

Embodiment 64. The method of embodiment 63, wherein the risk of severe bleeding of the first population of patients who are administered the antibody or antigen-binding fragment thereof after receiving ticagrelor is about 17.7 to about 29.0 lower than that of the second population of patients who is not administered the antibody or antigen-binding fragment thereof after receiving ticagrelor.

Embodiment 65. The method of embodiment 63, wherein the urgent surgery is CABG, and wherein the risk of severe bleeding of the first population of patients who are administered the antibody or antigen-binding fragment thereof after receiving ticagrelor is about 14.8 to about 31.6 lower than that of the second population of patients who are not administered the antibody or antigen-binding fragment thereof after receiving ticagrelor; optionally wherein the risk of severe bleeding of the first population of patients who are administered the antibody or antigen-binding fragment thereof after receiving ticagrelor is about 22.5 lower than that of the second population of patients who are not administered the antibody or antigen-binding fragment thereof after receiving ticagrelor.

Embodiment 66. The method of any one of embodiments 21-59, wherein a first patient who is administered the antibody or antigen-binding fragment thereof after receiving ticagrelor has a lower risk of mortality compared to a second patient who is not administered the antibody or antigen-binding fragment thereof after receiving ticagrelor, wherein the first patient and the second patient both have major bleeding; optionally, the major bleeding is procedure-related bleeding, non-procedure-related or spontaneous major bleeding, non-procedure-related or spontaneous intracerebral hematoma (ICH), or non-procedure-related or spontaneous non-ICH.

Embodiment 67. The method of embodiment 66, wherein the risk of mortality of the first patient who is administered the antibody or antigen-binding fragment after receiving ticagrelor is about 1.0 to about 16.4 lower than that of the second patient who is not administered the antibody or antigen-binding fragment thereof after receiving ticagrelor; optionally wherein the risk of mortality of the first patient who is administered the antibody or antigen-binding fragment after receiving ticagrelor is about 7.7 lower than that of the second patient who is not administered the antibody or antigen binding fragment thereof after receiving ticagrelor.

Embodiment 68. The method of any one of embodiments 21-59, wherein a first population of patients with major bleeding and who are administered the antibody or antigen-binding fragment thereof after receiving ticagrelor has a lower risk of mortality compared to a second population of patients with major bleeding and who are not administered the antibody or antigen-binding fragment thereof after receiving ticagrelor.

Embodiment 69. The method of embodiment 68, wherein the risk of mortality of the first population of patients who is administered the antibody or antigen-binding fragment thereof after receiving ticagrelor is about 1.0 to about 16.4 lower than that of the second population of patients who is not administered the antibody or antigen-binding fragment thereof after receiving ticagrelor.

Embodiment 70. The method of embodiment 66 or 67, wherein the antibody or antigen-binding fragment thereof is administered to the first patient during surgery or during an invasive procedure.

Embodiment 71. The method of any one of embodiments 21-65, wherein a first patient who is administered the antibody or antigen-binding fragment thereof after receiving ticagrelor has a lower risk of mortality compared to a second patient who is not administered the antibody or antigen-binding fragment thereof after receiving ticagrelor, wherein the first patient and the second patient both require urgent surgery.

Embodiment 72. The method of embodiment 71, wherein the risk of mortality of the first patient who is administered the antibody or antigen-binding fragment thereof after receiving ticagrelor is at least about 1.3 to about 11.4 lower than that of a second patient who is not administered the antibody or antigen-binding fragment thereof after receiving ticagrelor; optionally wherein the risk of mortality of the first patient who is administered the antibody or antigen-binding fragment thereof after receiving ticagrelor is about 6.6 lower than that of the second patient who is not administered the antibody or antigen-binding fragment thereof after receiving ticagrelor.

Embodiment 73. The method of any one of embodiments 21-65, wherein a first population of patients requiring urgent surgery and who are administered the antibody or antigen-binding fragment thereof after receiving ticagrelor has a lower risk of mortality compared to a second population of patients requiring urgent surgery and who are not administered the antibody or antigen-binding fragment thereof after receiving ticagrelor.

Embodiment 74. The method of embodiment 73, wherein the risk of mortality of the first population of patients who are administered the antibody or antigen-binding fragment thereof after receiving ticagrelor is at least about 1.3 to about 11.4 lower than that of the second population of patients who are not administered the antibody or antigen-binding fragment thereof after receiving ticagrelor; optionally wherein the risk of mortality of the first population of patients who are administered the antibody or antigen-binding fragment thereof after receiving ticagrelor is about 6.6 lower than that of the second population of patients who are not administered the antibody or antigen-binding fragment thereof after receiving ticagrelor.

Embodiment 75. The method of any one of embodiments 21-65, wherein a first patient who is administered the antibody or antigen-binding fragment thereof after receiving ticagrelor has a lower risk of mortality compared to a second patient who is not administered the antibody or antigen-binding fragment thereof after receiving ticagrelor, wherein the first patient and the second patient require urgent surgery or have major bleeding.

Embodiment 76. The method of embodiment 75, wherein the risk of mortality of the first patient who is administered the antibody or antigen-binding fragment thereof after receiving ticagrelor is at least about 0.5 to about 9.5 lower than that of the second patient who is not administered the antibody or antigen-binding fragment thereof after receiving ticagrelor; optionally wherein the risk of mortality of the first patient who is administered the antibody or antigen-binding fragment thereof after receiving ticagrelor is about 5.4 lower than that of the second patient who is not administered the antibody or antigen-binding fragment thereof after receiving ticagrelor.

Embodiment 77. The method of any one of embodiments 1-65, wherein a first population of patients who are administered the antibody or antigen-binding fragment thereof after receiving ticagrelor has a lower risk of mortality compared to a second population of patients who are not administered the antibody or antigen-binding fragment thereof after receiving ticagrelor, wherein the first population of patients and the second population of patients require urgent surgery or have major bleeding.

Embodiment 78. The method of embodiment 77, wherein the risk of mortality of the first population of patients who are administered the antibody or antigen-binding fragment thereof after receiving ticagrelor is at least about 0.5 to about 9.5 lower than that of the second population of patients who are not administered the antibody or antigen-binding fragment thereof after receiving ticagrelor; optionally wherein the risk of mortality of the first population of patients who are administered the antibody or antigen-binding fragment thereof after receiving ticagrelor is about 5.4 lower than that of the second population of patients who are not administered the antibody or antigen-binding fragment thereof after receiving ticagrelor.

Embodiment 79. The method of any one of embodiments 66-78, wherein the mortality is due to any cause within 38 days after start of major bleeding or within 38 days after start of urgent surgery.

Embodiment 80. The method of any one of embodiments 21-59, wherein a first patient or first population of patients who is administered the antibody or antigen-binding fragment thereof after receiving ticagrelor has a lower risk of receiving a first transfusion compared to a second patient or second population of patients who is not administered the antibody or antigen-binding fragment thereof after receiving ticagrelor, wherein the first and the second patient or first and second population of patients both have major bleeding.

Embodiment 81. The method of embodiment 80, wherein the risk ratio comparing the risk of receiving a first transfusion in the first patient or first population of patients who is administered the antibody or antigen-binding fragment thereof after receiving ticagrelor to the second patient or second population of patients who is not administered the antibody or antigen-binding fragment thereof after receiving ticagrelor is about 0.105 to about 0.413; optionally, wherein the risk ratio is about 0.259.

Embodiment 82. The method of embodiment 80, wherein the major bleeding is procedure-related major bleeding, and wherein the risk ratio comparing the risk of receiving a first transfusion in the first patient or first population of patients who is administered the antibody or antigen-binding fragment thereof after receiving ticagrelor to the second patient or second population of patients who is not administered the antibody or antigen-binding fragment thereof after receiving ticagrelor is about 0.286 to about 0.800; optionally, wherein the risk ratio is about 0.543.

Embodiment 83. The method of embodiment 80, wherein the major bleeding is spontaneous major bleeding, and wherein the risk ratio comparing the risk of receiving a first transfusion in the first patient or first population of patients who is administered the antibody or antigen-binding fragment thereof after receiving ticagrelor to the second patient or second population of patients who is not administered the antibody or antigen-binding fragment thereof after receiving ticagrelor is about −0.045 to about 0.190; optionally, wherein the risk ratio is about 0.072.

Embodiment 84. The method of embodiment 83, wherein the spontaneous major bleeding is non-intracranial hemorrhage (non-ICH) spontaneous major bleeding or ICH spontaneous major bleeding.

Embodiment 85. The method of any one of embodiments 21-59, wherein the method comprises:
i) administering to the patient or population of patients the antibody or antigen-binding fragment thereof, then
ii) administering a transfusion to the patient or population of patients at least 10 hours after (i) administering the antibody or antigen-binding fragment thereof.

Embodiment 86. The method of any one of embodiments 21-59, wherein the method increases the time between receiving bentracimab and receiving a first transfusion in a first patient or first population of patients who is administered the antibody or antigen-binding fragment thereof after receiving ticagrelor compared to the time between an equivalent start and receiving a first transfusion in a second patient or second population of patients who is not administered the antibody or antigen-binding fragment thereof after receiving ticagrelor, wherein the first and second patient or first and second population of patients both have major bleeding.

Embodiment 87. The method of any one of embodiments 21-59, wherein the method reduces the number of transfusions within 120 hours after start of major bleeding received by a first patient or first population of patients who is administered the antibody or antigen-binding fragment thereof after receiving ticagrelor compared to the number of transfusions within 120 hours after start of major bleeding received by a second patient or second population of patients who is not administered the antibody or antigen-binding fragment thereof after receiving ticagrelor.

Embodiment 88. The method of embodiment 80-82 or 85-87, wherein the antibody or antigen-binding fragment thereof is administered to the first patient or first population of patients during surgery or during an invasive procedure.

Embodiment 89. The method of any one of embodiments 21-59, wherein the method reduces the number of transfusions within 120 hours after start of urgent surgery received by a first patient or first population of patients who is administered the antibody or antigen-binding fragment thereof after receiving ticagrelor compared to the number of transfusions within 120 hours after start of urgent surgery received by a second patient or second population of patients who is not administered the antibody or antigen-binding fragment thereof after receiving ticagrelor.

Embodiment 90. The method of any one of embodiments 21-59, wherein the method comprises:
i) administering the antibody or antigen-binding fragment thereof that binds to ticagrelor to a population of patients, wherein the population of patients have major bleeding or require urgent surgery, then
ii) administering no more than a mean of 1 transfusion to the population of patients within 120 hours after start of surgery in the population of patients requiring urgent surgery or start of major bleeding in the population of patients who had major bleeding.

Embodiment 91. The method of any one of embodiments 21-59, wherein the method reduces the risk of requiring surgery to treat bleeding within 48 hours after an urgent surgery in a first patient or first population of patients who is administered the antibody or antigen-binding fragment thereof after receiving ticagrelor compared to the risk of requiring surgery to treat bleeding within 48 hours after an urgent surgery in a second patient or second population of patients who is not administered the antibody or antigen-binding fragment thereof after receiving ticagrelor.

Embodiment 92. The method of any one of embodiments 21-59, wherein the method reduces the risk of requiring surgery to treat bleeding within 48 hours after the start of major bleeding in a first patient or first population of patients who is administered the antibody or antigen-binding fragment thereof after receiving ticagrelor compared to the risk of requiring surgery to treat bleeding within 48 hours after the start of major bleeding event in a second patient or second population of patients who is not administered the antibody or antigen-binding fragment thereof after receiving ticagrelor; optionally wherein the risk ratio of comparing the risk of requiring surgery to treat bleeding within 48 hours after the start of major bleeding in a first patient or first population of patients who is administered the antibody or antigen-binding fragment thereof after receiving ticagrelor compared that of the second patient or second population of patients who is not administered the antibody or antigen-binding fragment thereof after receiving ticagrelor is about −0.051 to about 0.783; optionally, wherein the risk ratio is about 0.366.

Embodiment 93. The method of any one of embodiments 1-92, wherein administration of the antibody or antigen-binding fragment thereof to a population of patients results in a treatment-emergent adverse event related to the administration of the antibody or antigen-binding fragment thereof in less than about 0.5% of patients within the population of patients, wherein the treatment-emergent adverse event requires discontinuation of administration of the antibody or antigen-binding fragment thereof.

Embodiment 94. The method of any one of embodiments 1-93, wherein administration of the antibody or antigen-binding fragment thereof to a population of patients results in a treatment-emergent adverse event related to the administration of the antibody or antigen-binding fragment thereof resulting in death in less than about 5% of patients within the population of patients.

Embodiment 95. The method of any one of embodiments 1-94, comprising performing surgery or an invasive procedure on the patient, wherein the antibody or antigen binding fragment thereof is administered during surgery.

Embodiment 96. The method of any one of claims 1-95, wherein the antibody or antigen binding fragment thereof is administered at a concentration of about 100 mg/mL.

Embodiment 97. The method of any one of claims 1-96, wherein platelet function returns to normal within 5-10 minutes of administration of the antibody or antigen binding fragment thereof.

Embodiment 98. An article of manufacture comprising a vial comprising a monoclonal antibody fragment that binds to ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-hydroxyethoxy)cyclopentane-1,2-diol) or a metabolite or derivative thereof, wherein the monoclonal antibody comprises a variable heavy chain region (VH) comprising the amino acid sequence set forth in SEQ ID NO:01 and a variable light chain region (VL) comprising the amino acid sequence set forth in SEQ ID NO:02, wherein the vial comprises about 6 g of the monoclonal antibody fragment.

Embodiment 99. The article of manufacture of claim 98, wherein the article of manufacture comprises three vials, each vial comprising about 6 g of the monoclonal antibody fragment.

Embodiment 100. The article of manufacture of embodiment 98 or 99, wherein the monoclonal antibody fragment is in a pharmaceutical composition at a concentration of about 100 mg/mL.

Embodiment 101. The article of manufacture of any one of embodiments 98-100, comprising instructions for treating patients requiring nondeferrable surgery or invasive procedure or in patients experiencing major bleeding.

Embodiment 102. The article of manufacture of any one of embodiments 98-101, wherein the article of manufacture is a carton comprising an 18 g treatment course comprising three vials of the monoclonal antibody fragment.

### EXAMPLES

### Example 1: A Phase 3, Multicenter, Open-Label, Single-Arm Study of Bentracimab in Ticagrelor-Treated Patients with Uncontrolled Major or Life-Threatening Bleeding or Requiring Urgent Surgery or Invasive Procedure

This Example describes a Phase 3, multicenter, open-label, prospective single-arm study of reversal of the antiplatelet effects of ticagrelor with bentracimab in patients who present with uncontrolled major or life-threatening bleeding or who require urgent surgery or invasive procedure.

### Protocol

At least 200 patients were enrolled in a phase III clinical trial testing efficacy of bentracimab for reversing ticagrelor-enhanced bleeding.

Bentracimab is a ticagrelor-specific human monoclonal antibody fragment which binds to ticagrelor and the ticagrelor active metabolite (TAM) with high affinity, thereby reversing the antiplatelet effects of ticagrelor. Bentracimab has no known off-target effects. The U.S. Food and Drug Administration granted Breakthrough Therapy designation to bentracimab in 2019, acknowledging that the preliminary clinical evidence indicates the drug may demonstrate substantial improvement over existing therapies. Bentracimab comprises the amino acid sequences set forth in Table 1.

Patients with reported use of ticagrelor within the prior 3 days who require urgent ticagrelor reversal were eligible for enrollment.

These patients fell into two major groups: 1) patients with uncontrolled major or life-threatening bleeding in whom the extent of hemostasis and the time to effective hemostasis after initiation of bentracimab were determined; and 2) patients undergoing urgent surgery or invasive procedure in whom the extent of procedure-related hemostasis after initiation of bentracimab were determined. The group of patients with uncontrolled major or life-threatening bleeding included patients who started receiving bentracimab during surgery.

Intravenous (IV) infusion of bentracimab was administered as follows:
An initial intravenous bolus of 6 g was infused over 10 minutes for rapid reversal followed immediately by a 6 g loading infusion infused over 4 hours.
A 6 g maintenance infusion over 12 hours immediately followed the 4-hour loading infusion for a total infusion time of 16 hours and 10 minutes.

If extended reversal was needed based on signs or symptoms of ongoing hemorrhage or risk of re-bleeding, the maintenance infusion of bentracimab was extended with an additional 6 g of bentracimab infused for 12 more hours. If extended reversal is needed based on signs or symptoms of ongoing hemorrhage or risk of re-bleeding, the duration of the bentracimab maintenance infusion may be extended to provide an additional 12-18 hours of reversal time for each additional 6 g (60 mL) infused. The additional administration of 6 g over 12 hours may be repeated as necessary.

In patients taking ticagrelor who have recently taken a moderate or strong CYP3A inhibitor, such as, diltiazem or verapamil, an alternative regimen of bentracimab was administered to address the potential for a significant drug interaction if the initiation of bentracimab occurs within 5 half-lives of the last CYP3A inhibitor dose. This alternative regimen was administered as follows:
An initial 12 g bolus infusion over 10 minutes followed immediately by a loading infusion of 12 g over 6 hours.
A 12 g maintenance infusion over 18 hours immediately followed the 6-hour loading regimen for a total infusion time of 24 hours and 10 minutes.

If extended reversal was needed based on signs or symptoms of ongoing hemorrhage or risk of re-bleeding, the maintenance infusion of bentracimab was extended with an additional 12 g in subjects who have recently taken a moderate or strong CYP3A inhibitor of bentracimab infused for 18 more hours in subjects who have recently taken moderate or strong CYP3A inhibitor. If extended reversal is needed based on signs or symptoms of ongoing hemorrhage or risk of re-bleeding, the duration of the bentracimab maintenance infusion may be extended to provide an additional 12-18 hours of reversal time for each additional 6 g (60 mL) infused. The additional administration of 12 g over 18 hours in subjects who have recently taken moderate or strong CYP3A inhibitor may be repeated as necessary.

Bentracimab was formulated as a 100 mg/mL solution in 25 mM L-histidine/L-histidine HCl monohydrate, 290 mM sucrose, 0.05% polysorbate 80, pH 6.0. For this study, bentracimab was supplied as a high-concentration liquid. A total 18 g intravenous infusion will consist of an initial IV bolus of 6 g (60 mL) infused over 10 minutes for rapid reversal. A 6 g (60 mL) loading dose will then be infused and administered over 4 hours immediately following the bolus infusion, followed by a maintenance dose of 6 g (60 ml) infused over the next 12 hours immediately following completion of the loading dose for a total dose of 18 g (180 mL) and infusion time of 16 hours and 10 minutes. Bentracimab was delivered via a standard IV bag(s) and IV pump, or by syringe using a syringe pump.

For patients in need of urgent surgery or invasive procedure, bentracimab infusion was initiated no more than 16 hours prior to the urgent surgery or invasive procedure.

Upon enrollment, samples were collected pre-reversal for assessment of baseline platelet function, baseline hemoglobin levels, and baseline pharmacokinetics (PK) of ticagrelor, its active metabolite TAM, and bentracimab. Additional platelet function, hematology, and PK sample collections occurred 5-10 minutes after initiation of the bentracimab bolus infusion and at multiple prespecified timepoints thereafter through 24 hours to support assessments of ticagrelor reversal and hemostasis.

In bleeding patients, hemostasis was initially assessed using all available standard of care data, such as, cessation of visible bleeding, amelioration of pain and swelling at the bleeding site, imaging results, etc. collected through 24 hours from the initiation of bentracimab, and results from serial assessments of hemoglobin (or corrected hemoglobin) at multiple prespecified timepoints following initiation of bentracimab if needed. In patients presenting with intracranial hemorrhage (ICH), brain imaging within 2 hours of initiation of bentracimab and at least one follow-up brain imaging performed 12-24 hours post completion of bentracimab were required to support adjudication of hemostasis.

In patients requiring urgent surgery or invasive procedure, hemostasis was assessed initially using all available perioperative data related to bleeding that occurs during the first 24 hours after initiation of bentracimab. Perioperative hemostasis was assessed as normal or abnormal for the type of surgery or procedure performed, and if abnormal the extent of abnormality should be assessed as mild (slight oozing), moderate (controllable bleeding), or severe (severe refractory hemorrhage).

To determine achievement of primary endpoints, ticagrelor reversal was based on reduction of percent (%) inhibition of platelet function as measured by the VerifyNow™ PRUTest™ assay within 4 hours of initiation of bentracimab. Achievement of effective hemostasis was centrally adjudicated and based on pooled analysis of the extent of hemostasis achieved in the bleeding and surgical patients.

Other clinical endpoints related to clinical support of enrolled subjects, such as, days in hospital, days in intensive care (if any), surgical and other pharmacologic actions taken to control bleeding, quantity and type of blood products transfused, restart of ticagrelor or other antiplatelet agent, and recurrent bleeding events were analyzed. Data related to physician use of ticagrelor after reversal, e.g., permanent discontinuation or immediate or delayed resumption of ticagrelor use post-completion of bentracimab infusion, were also analyzed. Patients enrolled with ICH as either urgent surgery or bleeding patients were asked to participate in a 90-day ICH-only follow-up visit to assess functional status with the modified Rankin Scale (mRS) and health-related quality of life (QoL) with the EQ-5D 5L QoL questionnaire (standard-of-care mRS and EQ-5D 5L assessments may be used).

Safety monitoring of all treatment emergent adverse events (TEAEs), including serious adverse events (SAEs) and mortality, occurred for 35 days following initiation of bentracimab. Post-reversal thrombotic events, including major adverse cardiovascular events (MACE), other systemic thromboembolic events, and venous thromboembolism occurring during the 35-day safety follow-up period were considered AEs of Special Interest (AESI).

### Objectives and Endpoints

This was an open-label, single-cohort study. The primary objectives were to demonstrate reversal of the antiplatelet effects of ticagrelor with IV infusion of bentracimab and to demonstrate the clinical efficacy of bentracimab by assessment of hemostasis in ticagrelor-treated patients with uncontrolled major or life-threatening bleeding or who were undergoing urgent surgery or invasive procedure.

### Primary Objectives and Endpoints

### Primary Objectives Included:

To demonstrate reversal of the antiplatelet effects of ticagrelor after initiation of the intravenous (IV) infusion of bentracimab using the VerifyNow™ PRUTest™ (VerifyNow™. (2016). VerifyNow™ PRU Test Instructions for Use. San Diego, CA: Accriva Diagnostics. Retrieved Jan. 23, 2019, from http://www.accriva.com/uploads/literature/vn1016en-web-01.pdf) platelet function assay in ticagrelor-treated patients presenting with uncontrolled major or life-threatening bleeding or requiring urgent surgery or invasive procedure
To demonstrate the effect of bentracimab on achievement of effective hemostasis after administration of bentracimab in ticagrelor-treated patients presenting with uncontrolled major or life-threatening bleeding or requiring urgent surgery or invasive procedure

Primary Reversal Endpoint: The minimum % inhibition of PRU within 4 hours of the initiation of bentracimab as assessed by the VerifyNow™ PRUTest™ platelet function assay. Percent inhibition of PRU was calculated as 100×[(180−PRUₜᵣₜ)/180]. PRUₜᵣₜ refers to the PRU value measured posttreatment with the bentracimab. PRU of 180 was considered the lower limit of normal (LLN) platelet function as described in the VerifyNow™ PRUTest™ manufacturer's user guidance.

Primary Hemostasis Endpoint: Achievement of effective hemostasis after initiation of bentracimab infusion were assessed in each population separately and then pooled for primary endpoint analysis. In patients with uncontrolled major bleeding, achievement of effective hemostasis was centrally adjudicated using prespecified criteria for effective hemostasis for visible and non-visible major bleeding adapted from (Connolly, S. M., et al. (2016). N Engl J Med, 375, 1131-1141). In patients undergoing urgent surgery or invasive procedure, achievement of effective hemostasis following initiation of bentracimab infusion were determined using prespecified criteria for effective hemostasis derived from the GUSTO (Global Utilization of Streptokinase and Tpa For Occluded Arteries) clinical bleeding scale. See, GUSTO. (1993). N Engl J Med, 673-82.

### Secondary Objectives and Endpoints

A secondary objective of the study was to demonstrate reversal of the antiplatelet effects of ticagrelor with intravenous infusion of bentracimab by measurement of the platelet reactivity index (PRI) using the vasodilator-stimulated phosphoprotein (VASP) assay in addition to PRU in ticagrelor-treated patients presenting with uncontrolled major or life-threatening bleeding or requiring urgent surgery or invasive procedure.

Secondary endpoints of the study included:
Minimum % inhibition of PRI assessed by VASP within 4 hours after the initiation of bentracimab. Percent inhibition of PRI was calculated as 100*[(PRI_{bsl}−PRIₜᵣₜ)/PRI_{bsl}]. PRIₜᵣₜ refers to the PRI value measured posttreatment with the bentracimab. PRI_{bsl} was the lower limit of normal obtained from previous studies in healthy volunteers.
Maximum reversal of PRU assessed by VerifyNow™ PRUTest™ within 4 hours after the initiation of bentracimab. Percent reversal was calculated as 100*[(PRUₜᵣₜ−PRUₚᵣₑ−trt)/(180−PRUₚᵣₑ₋ₜᵣₜ)]. PRUₚᵣₑ₋ₜᵣₜ was defined as the PRU value prior to administration of bentracimab. PRU of 180 was considered as the lower limit of normal (LLN) for platelet function as described in the VerifyNow™ PRUTest™ package insert.
Maximum reversal of PRI assessed by VASP within 4 hours after the initiation of bentracimab. Percent reversal was calculated as 100*[(PRIₜᵣₜ−PRIₚᵣₑ₋ₜᵣₜ)/(LLN−PRIₚᵣₑ₋ₜᵣₜ)]. PRIₚᵣₑ₋ₜᵣₜ was defined as the PRI value prior to administration of bentracimab. LLN of PRI was obtained from previous studies in healthy volunteers.
Proportion of subjects achieving ≥lower limit of normal, ≥60%, ≥80% or ≥100% reversal of platelet inhibition by ticagrelor using PRU and PRI at any time point during the treatment period. Achievement of PRU or PRI≥lower limit of normal indications normalization of platelet function.
Duration of achieving ≥lower limit of normal, ≥60%, ≥80%, and ≥100% reversal of PRU and PRI

### Other Prespecified Objectives and Endpoints

Other prespecified objectives of the study included:
To assess clinical outcomes in all enrolled patients dosed with bentracimab.
To evaluate the pharmacokinetic (PK) profile of intravenous bentracimab, ticagrelor, and the ticagrelor active metabolite (TAM)
To assess functional and patient reported outcomes (PROs) in all enrolled ICH patients who agree to participate in the ICH-only 90-day follow-up visit.
To assess the effect of ticagrelor reversal on P-selectin and optionally other platelet function-related biomarkers

Other prespecified endpoints of the study included:
Time to effective hemostasis from the initiation of bentracimab infusion
Days in hospital and days in intensive care (if any)
Quantity, type, and timing of blood products transfused.
Quantity, type, and timing of non-blood products administered to support hemostasis.
Proportion of subjects restarting ticagrelor administered 60 mg BID or 90 mg BID and time to restart of ticagrelor post-reversal if restarted within 35+3 days.
Proportion of subjects re-hospitalized for bleeding after initial post-reversal discharge and time to rehospitalization post-reversal.
Proportion of subjects who require urgent surgery or other procedure to treat a bleeding-related condition after initiation of bentracimab infusion and time to urgent surgery or intervention for bleeding-related condition post-initiation of bentracimab infusion.
Degree of perioperative bleeding in surgical/procedure patients
Time to 60%, 80%, 100% reversal by PRU and PRI within 4 hours after initiation of bentracimab
Plasma concentrations of total bentracimab, unbound bentracimab, total ticagrelor and its active metabolite TAM, unbound ticagrelor, and unbound TAM at predetermined timepoints
Circulating levels of P-selectin and optionally other platelet function-related biomarkers at baseline and post-initiation of bentracimab
ICH only: Proportion of ICH patients with modified Rankin Scale (mRS) score of 0-3 versus 4-6 at 90 days
ICH only: Absolute and percent change form baseline in mRS score at 90 days in ICH patients.
ICH only: EQ-5D 5L index at 90 days and change from baseline in ICH patients.

A safety objective of the study was to evaluate the safety, tolerability, and immunogenicity of bentracimab in ticagrelor-treated patients.

Safety endpoints of the study included:
Safety and tolerability were assessed by monitoring and recording of vital status, AEs, vital signs, and clinical laboratory assessments through 35+3 days post-initiation of bentracimab infusion. All SAEs were centrally adjudicated.
Post-reversal thrombotic events, including major adverse cardiovascular events (MACE), other systemic thromboembolic events, and venous thromboembolism occurring during the 35-day safety follow-up period were considered AEs of Special Interest (AESI) and were centrally adjudicated
Immunogenicity response to bentracimab

### Inclusion/Exclusion Criteria

Patients were eligible for inclusion into the study if they met all of the following criteria:
1. Male or female >18 years of age with documented or verbal informed consent. Emergency consent may be obtained where permitted by local regulations and institutional approval.
2. History or documentation of ticagrelor intake within the prior 3 days.
3. Patients described below who require urgent reversal of the antiplatelet effects of ticagrelor:

Patients with uncontrolled major or life-threatening bleeding, requiring urgent reversal of the antiplatelet effects of ticagrelor. It was expected that enrolled patients would have characteristics similar to those described below:
Potentially life-threatening bleeding with signs or symptoms of hemodynamic compromise, e.g., systolic blood pressure <90 mm Hg and signs or symptoms of low cardiac output not otherwise explained.
Bleeding in a critical organ or closed space, such as intracranial, intraspinal, intraocular, retroperitoneal, intra-articular, pericardial, or intramuscular bleed with compartment syndrome
Uncontrolled bleeding associated with a corrected hemoglobin level <8.0 g/dL, a fall in hemoglobin level of ≥2.0 g/dL (1.24 mmol/L) from a known baseline, or transfusion of 2 or more units of packed red blood cells (PRBC)
Visible, uncontrolled bleeding associated with a corrected hemoglobin level <8.0 g/dL, a fall in hemoglobin level of ≥2.0 g/dL (1.24 mmol/L) from a known baseline, or requirement for transfusion of 2 or more units of packed red blood cells (PRBC)

Patients requiring urgent surgery or invasive procedure when it was not medically advisable either to proceed urgently with impaired hemostasis or to delay the urgent procedure for 3 or more days due to the high risk of bleeding. These patients may typically be in any of the following clinical situations:
Requires urgent surgery or invasive procedure known to be associated with a risk of significant bleeding (such as cardiac surgery, neurosurgery, or major orthopedic surgery)
Requires urgent surgery or invasive procedure that may have an adverse procedural outcome if hemostasis was impaired (such as neurological, spinal, ophthalmological, urological, or orthopedic surgery)
   At risk of experiencing life-threatening events, such as, shock, myocardial infarction, or stroke, if significant intraoperative or postoperative bleeding occurs (such as in elderly patients or patients with underlying cardiac or pulmonary disease who have limited cardiopulmonary reserve)

Exclusion criteria included:
1. Known sensitivity or contraindication to bentracimab or any of its excipients.
2. Patients in whom ticagrelor reversal was not considered urgent, e.g., patients with stable or non-acute conditions who have low hemoglobin due to chronic, low-grade gastrointestinal bleeding or who have stable, remote, or asymptomatic intracranial hemorrhage.
3. Patients expected to be clinically unsalvageable, such as patients with end-stage cancer or patients with overwhelming sepsis.
4. Any condition which would make it unsafe or unsuitable for the patients to participate in this study. This includes assessment of likelihood to cooperate with study follow-up visits and procedures.
   Known pregnancy may be exclusionary in some regions or countries as directed by national health authorities and/or local IRBs/Ethics Committees
5. Known use of clopidogrel, prasugrel, or ticlopidine within 5 days of bentracimab administration; known use of antiplatelet GPIIb/IIIa inhibitors, or cangrelor within 5 half-lives of expected bentracimab administration; or known use of warfarin, dabigatran, rivaroxaban, apixaban, or edoxaban within 5 half-lives of expected bentracimab administration
6. Known recent use (<5 day) of vitamin K, prothrombin complex concentrate, recombinant factor VIIa, idarucizumab, or andexanet-alfa (coagulation factor Xa (recombinant), inactivated-zhzo)

### Statistical Analysis

### Sample Size:

At least 200 patients were enrolled into the final analysis population which were comprised of ticagrelor-treated patients who require urgent surgery or invasive procedure.

For assessment of the primary reversal endpoint, a sample size of 30 subjects in either population provides 90% power to detect a statistically significant decrease of at least 24.5% in % inhibition of PRU using the VerifyNow™ PRUTest™ assay. Detection of this degree of ticagrelor reversal assumes a standard deviation of 40% when comparing the mean difference between the minimum % inhibition of PRU within 4 hours post-dose compared to the pre-dose value, using a 2-sided test with a type I error rate of 0.05.

For assessment of the primary hemostasis endpoint, a sample size of 150 evaluable subjects provides 90% power to detect achievement of effective hemostasis in at least 63.4% of the total pooled study population compared to 50%, using an exact 2-sided test for a proportion with alpha=0.05.

### Safety:

Safety data were presented using descriptive statistics: mean, SD, median, min, and max for continuous variables; count and percentage for categorical variables.

### Interim Analysis

Table 4 shows the demographics of the enrolled patients. The interim analysis included 191 unique enrolled (dosed) patients, of which 180 were eligible, and of those, 136 were patients requiring surgery or invasive procedure (surgery group) and 44 were patients with major or life-threatening bleeding (major bleeding group). There was a 6.3% early termination rate.

TABLE 4
Disposition of enrolled subjects

| | Enrolled | | Enrolled Eligible | | | |
|---|---|---|---|---|---|---|
| | Total | | Surgery | | Major Bleeding | |
| | N = 192¹ | | (N = 136) | | (N = 44) | |
| Enrolled (dosed) | 192 | (100%) | 136 | (100%) | 44 | (100%) |
| Completed study | 183 | (95.3%) | 132 | (97.1%) | 40 | (90.1%) |
| Terminated early | 9 | (6.3%) | 4 | (2.9%) | 4 | (9.1%) |
| Death | 8 | (4.2%) | 4 | (2.9%) | 4 | (9.1%) |

| | | | | |
|---|---|---|---|---|
| Physician decision | 1 | (0.5%) | 0 | 0 |

¹One patient was counted twice.

Table 5 shows the baseline demographics of the subjects included in the analysis described herein. Elderly white men were common study participants. The mean age of the total population was 65.6 years.

TABLE 5
Baseline Demographics

| | Enrolled | | Eligible Enrolled | | | |
|---|---|---|---|---|---|---|
| | Total | | Surgery | | Major Bleeding | |
| | (N = 191) | | (N = 136) | | (N = 44) | |
| Age, mean (SD) | 65.6 | (10.89) | 65.2 | (10.55) | 67.2 | (12.62) |
| Male, % | 150 | (78.5%) | 108 | (79.4%) | 33 | (75.0%) |
| BMI, kg/m², | 28.97 | (6.015) | 29.19 | (6.404) | 27.74 | (4.280) |
| mean (SD) | | | | | | |
| Hispanic or | 3 | (1.6%) | 1 | (0.7%) | 2 | (4.5%) |
| Latino, n (%) | | | | | | |
| Race, n, (%) | | | | | | |
| White | 162 | (84.8%) | 114 | (83.1%) | 39 | (88.6%) |
| Asian | 17 | (8.9%) | 16 | (11.3%) | 1 | (2.3%) |
| Black or African | 7 | (3.7%) | 4 | (2.9%) | 2 | (4.5%) |
| American | | | | | | |
| American Indian | 1 | (0.5%) | 1 | (0.7%) | 0 | |
| or Alaska Native | | | | | | |
| Other | 4 | (2.1%) | 1 | (0.7%) | 2 | (4.5%) |
| Region, n, (%) | | | | | | |
| North America | 154 | (80.6%) | 117 | (86.0%) | 31 | (70.5%) |
| United States | 37 | (19.4%) | 21 | (15.4%) | 12 | (27.3%) |
| Canada | 117 | (61.3%) | 96 | (70.6%) | 19 | (43.2%) |
| Europe | 37 | (19.4%) | 19 | (14.0%) | 13 | (29.5%) |

Table 6 shows the baseline diseases of the subjects. Study participants frequently had a history of significant underlying medical illnesses, such as myocardial infarctions, diabetes, hypertension, and renal impairment. Estimated glomerular filtration rate (eGFR) is a measure of the amount of creatinine in the blood, which is an indicator of kidney function. A normal eGFR is 60 or more. End-Stage Renal Disease (ESRD) is a medical condition in which a patient's kidneys cease functioning on a permanent basis leading to the need for a regular course of long-term dialysis or a kidney transplant to maintain life.

TABLE 6
Baseline Diseases

| Enrolled | Eligible Enrolled | |
|---|---|---|
| Total | Surgery | Major Bleeding |
| (N = 191) | (N = 136) | (N = 44) |

Renal Function

| Normal, eGFR ≥90 | 68 | (35.6%) | 53 | (39.0%) | 11 | (25.0%) |
|---|---|---|---|---|---|---|
| mL/min | | | | | | |
| Mild, eGFR ≥60 | 80 | (41.9%) | 60 | (44.1%) | 15 | (34.1%) |
| and ≤89 mL/min | | | | | | |
| Moderate, eGFR ≥30 | 32 | (16.8%) | 15 | (11.0%) | 16 | (36.4%) |
| and ≤59 mL/min | | | | | | |
| Mild-moderate, ≥45 | 21 | (11.0%) | 10 | (7.4%) | 10 | (22.7%) |
| and ≤59 mL/min | | | | | | |
| Moderate-severe, | 11 | (5.8%) | 5 | (3.7%) | 6 | (13.6%) |
| eGFR ≥30 and ≤44 | | | | | | |
| mL/min | | | | | | |
| Severe, eGFR ≥15 | 2 | (1.0%) | 2 | (1.5%) | 0 | |
| and ≤29 mL/min | | | | | | |
| ESRD, eGFR <15 | 5 | (2.6%) | 3 | (2.2%) | 2 | (4.5%) |
| mL/min | | | | | | |
| Prior Myocardial | 153 | (80.1%) | 117 | (86.0%) | 28 | (63.8%) |
| Infarction | | | | | | |
| Hypertension | 149 | (78.0%) | 108 | (79.4%) | 33 | (75.0%) |
| Diabetes | 82 | (42.9%) | 58 | (43.6%) | 20 | (45.5%) |
| Prior Stroke | 7 | (3.7%) | 4 | (2.9%) | 2 | (4.5%) |
| Prior Peripheral | 6 | (3.1%) | 2 | (1.5%) | 2 | (4.5%) |
| Vascular Disease | | | | | | |

Table 7 shows baseline ticagrelor and history of acetylsalicylic acid (ASA) use. Acute coronary syndrome (ACS) was a common reason for ticagrelor use. All subjects used ticagrelor within 3 days prior to screening, and 96.9% were also receiving treatment with acetylsalicylic acid. The population included patients who used ticagrelor for reasons including acute coronary syndrome, myocardial infarction (MI), unstable angina, percutaneous coronary intervention (PCI), coronary artery bypass grafting (CABG), and stroke.

TABLE 7
Baseline Ticagrelor and Acetylsalicyclic Acid (ASA) History

| | Enrolled | | Eligible Enrolled | | | |
|---|---|---|---|---|---|---|
| | Total | | Surgery | | Major Bleeding | |
| | (N = 191) | | (N = 136) | | (N = 44) | |
| Ticagrelor use in last 3 | 191 | (100%) | 136 | (100%) | 44 | (100%) |
| days prior to Screening, n | | | | | | |
| Ticagrelor daily dose, | 185.1 | (90, 360) | 186.0 | (90, 360) | 183.4 | (90, 360) |
| mean mg (range) | | | | | | |
| Days on ticagrelor, | 65.8 | (0, 3334) | 19.7 | (0, 483) | 235.4 | (0, 3334) |
| mean (range) | | | | | | |
| Days between last dose | 1.0 | (0.78) | 1.1 | (0.78) | 0.8 | (0.73) |
| of ticagrelor and start | | | | | | |
| of bentracimab infusion, | | | | | | |
| mean (SD) | | | | | | |
| Reasons for ticagrelor | | | | | | |
| use, n (%) | | | | | | |
| Acute Coronary Syndrome | 159 | (83.2%) | 124 | (91.2%) | 28 | (63.6%) |
| MI, suspected MI | 157 | (81.2%) | 123 | (90.4%) | 28 | (63.6%) |
| Unstable Angina | 2 | (1.0%) | 2 | (1.5%) | 0 | |
| PCI | 52 | (27.2%) | 24 | (17.6%) | 21 | (47.7%) |
| CABG | 10 | (5.2%) | 9 | (6.6%) | 1 | (2.3%) |
| Stroke | 2 | (1.0%) | 0 | | 1 | (2.3%) |
| Other | 4 | (2.1%) | 2 | (1.5%) | 2 | (4.5%) |
| ASA use, n (%) | 185 | (96.9%) | 133 | (97.8%) | 42 | (95.5%) |

The primary efficacy endpoint and secondary efficacy endpoints (as described in the above Protocol, Objectives and Endpoints section) were all met, for the total enrolled population and surgery and major bleeding groups, all p<0.0001 (paired t-test).

Table 8 shows the results of the primary endpoint analysis. The primary efficacy endpoint was met. The primary endpoint analysis was minimum % inhibition of P2Y₁₂ reaction units (PRU) over 4 hours after starting bentracimab administration versus Baseline, in total enrolled (dosed) population. PRU measures P2Y₁₂-mediated platelet aggregation. “Pre-dose” values in Table 8 indicate % inhibition PRUₚᵣₑ, which is calculated as 100×(180−PRUₚᵣₑ)/180. “Minimum over 4 hrs” values in Table 8 indicate % inhibition PRUₜᵣₜ, which is calculated as 100×(180−PRUₜᵣₜ)/180. PRUₚᵣₑ and PRUₜᵣₜ refer to the PRU value measured at pre-treatment and post-treatment with the bentracimab, respectively, wherein normal PRU is ≥180. The mean difference in minimum % inhibition of P2Y₁₂ reaction units (PRU) over 4 hours=[% inhibition PRUₜᵣₜ]−[% inhibition PRUₚᵣₑ].

Measurements were taken at baseline, 5-10 minutes, 30 minutes, 1 hour, and 4 hours after starting bentracimab infusion. The more negative the minimum % inhibition of PRU over 4 hours, the greater the effect of bentracimab on reversing ticagrelor's effects on platelet aggregation. Eligible enrolled population and its surgery and major bleeding groups were adjudicated by a Clinical Events Committee (CEC).

TABLE 8
Primary endpoint: Minimum % inhibition of P2Y₁₂ reaction units (PRU) over
4 hours after starting bentracimab administration versus Baseline

| | Enrolled | Eligible Enrolled | |
|---|---|---|---|
| | Total | Surgery | Major Bleeding |
| | (N = 191) | (N = 136) | (N = 44) |
| Mean Pre-dose | 50.79 | 51.54 | 49.03 |
| (95% CI) | (45.03, 56.55) | (44.77, 58.31) | (33.89, 62.17) |
| Mean Minimum | −71.153 | −73.03 | −66.68 |
| over 4 hrs (95% CI) | (−75.86, −67.20) | (−77.96, −68.11) | (−77.51, −55.84) |
| Mean Difference | −121.984 | −124.58 | −115.71 |
| (95% CI) | (−128.54, −115.34) | (−132.32, −116.83) | (−130.32, −101.09) |
| p-value (two-sided) | <.0001 | <.0001 | <.0001 |

Table 9 describes the proportion of subjects achieving effective hemostasis among total eligible subjects, which was a secondary efficacy endpoint. This secondary efficacy endpoint was met. The analysis measured the proportion of subjects achieving effective (excellent or good) hemostasis in total eligible enrolled population versus 50%. Wald method was used to calculate confidence intervals and P-value. Eligibility, surgery versus major bleeding group, and effectiveness of hemostasis adjudicated a Clinical Events Committee (CEC). The CEC used Gusto scoring for surgery or procedure group and the scoring system of Connelly et al. (2016) for major bleeding group. This analysis included “unable to determine” as not effective hemostasis.

TABLE 9
Proportion of Subjects Achieving Effective
Hemostasis among Total Eligible Subjects

| | Eligible Enrolled Subjects | | |
|---|---|---|---|
| | Total | Surgery | Major Bleeding |
| | (N = 180) | (N = 136) | (N = 44) |
| n (Proportion) | 171 | 136 | 35 |
| Proportion | 95.0% (91.8%, | 100% (100%, | 79.5% (67.6%, |
| (95% CI) | 98.2%) | 100%) | 91.5%) |
| p-value | <.0001 | <.0001 | <.0001 |

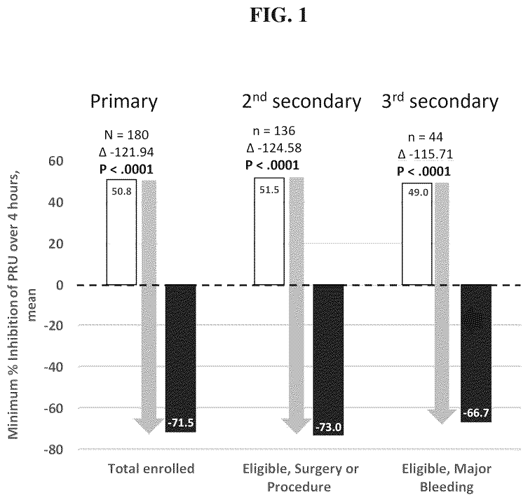

FIG. 1 shows that the minimum % inhibition of PRU significantly over 4 hours in total patient population (primary endpoint), patients requiring surgery or invasive procedure (surgery or procedure group; 2^{nd} secondary endpoint), and patients with major or life-threatening bleeding (major bleeding group; 3^{rd} secondary endpoint). The primary endpoint and these secondary efficacy endpoints were met. PRU measures P2Y₁₂-mediated platelet aggregation and is a measure of platelet aggregation. Inhibition of PRU measures bentracimab's neutralization of ticagrelor's inhibitor effects on platelet aggregation through P2Y₁₂. Percent inhibition of PRU at baseline in the total enrolled population is 50.8, while the minimum percent inhibition of PRU over 4 hours after start of administration of bentracimab is −71.5. Statistically significant differences (P<0.0001) were found in minimum % inhibition of PRU in 4-hours after starting bentracimab administration compared to baseline in 1) total enrolled (dosed) population, 2) patients requiring surgery or invasive procedure (surgery group), and 3) patients with major or life-threatening bleeding (major bleeding group). This shows that bentracimab was effective at reversing platelet aggregation inhibition within the enrolled patients, regardless of if bentracimab was administered prophylactically to a patient in requiring surgery or invasive procedure, or if bentracimab is administered to a patient who is already experiencing major procedure, e.g., during surgery.

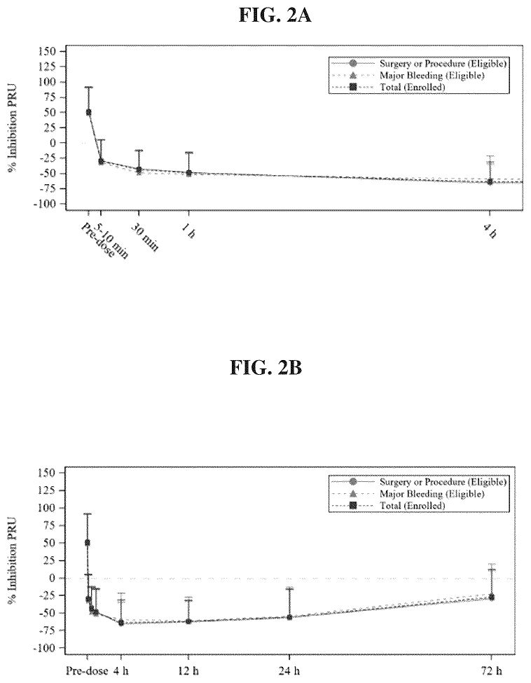

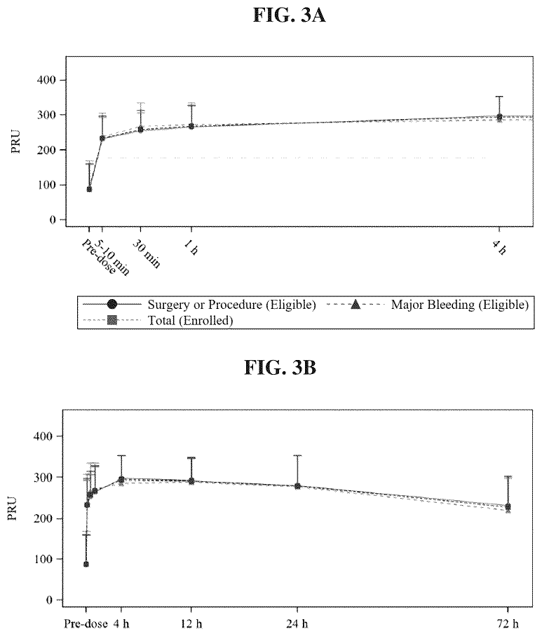

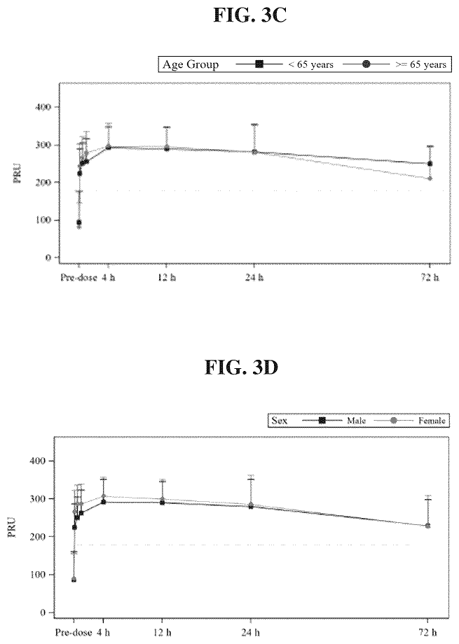

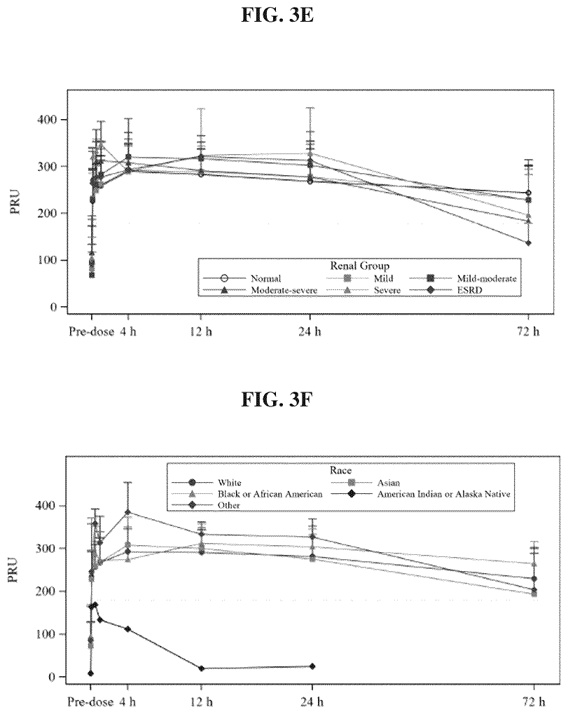

The results further demonstrate the ability of bentracimab to rapidly and durably reverse ticagrelor-enhanced bleeding. Normal P2Yu-mediated platelet aggregation (denoted by the dotted line) is quickly restored after the first 5-10 minutes after start of administration of bentracimab (FIG. 2A), and normal platelet aggregation is maintained up to at least 72 hours after start of administration of bentracimab (FIG. 2B). These results are also shown in terms of PRU levels FIG. 3A-3B, wherein PRU above the dotted line denotes at least normal P2Y₁₂-mediated platelet aggregation. Slight reduction in PRU at 72 hours is explained by minimal residual free ticagrelor in absence of free bentracimab.

Furthermore, the study showed that bentracimab was effective at normalizing platelet aggregation regardless of age group (FIG. 3C), sex (FIG. 3D), kidney disease status (FIG. 3E), and race (FIG. 3F).

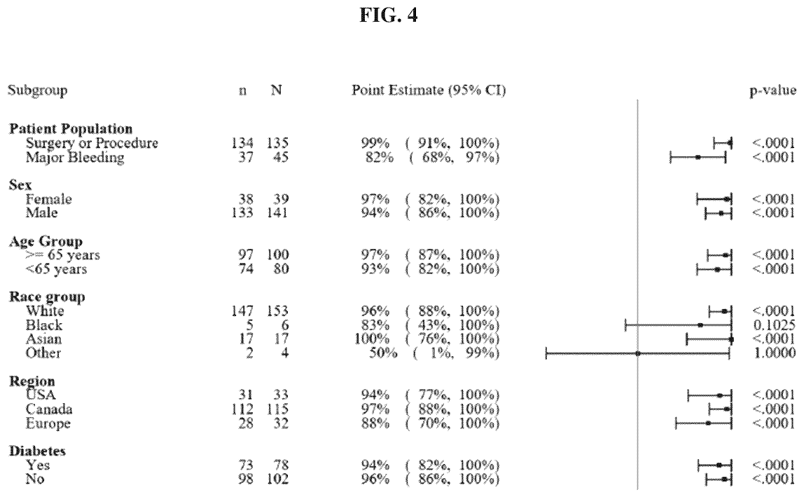

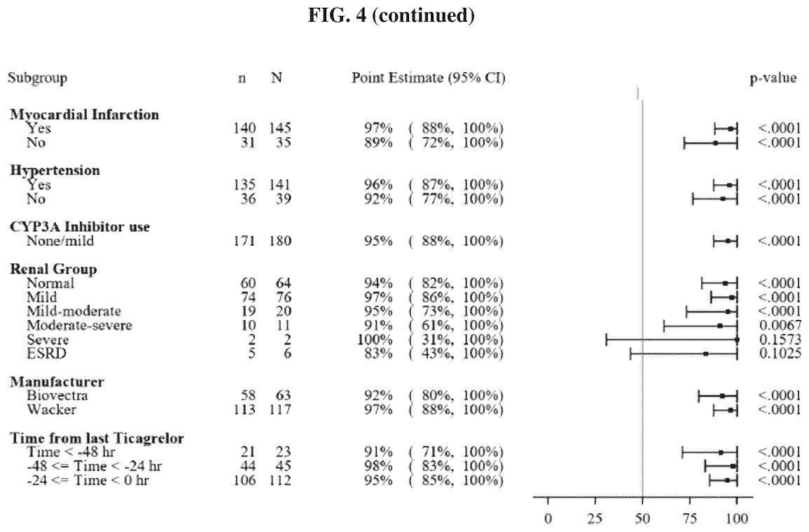

Forest plot analysis for minimum % inhibition of PRU further support the conclusion that bentracimab is effective at neutralizing ticagrelor's inhibitory effect on platelet aggregation, regardless of the patient has major bleeding or require surgery or an invasive procedure, sex, age, race, region, diabetes status, myocardial infarction history, hypertension status, CYP3A use, kidney disease status, manufacturer of ticagrelor used, or time from last ticagrelor dose (FIG. 4). Surprisingly, despite that patients who used ticagrelor within less than 24 hours were expected to have more ticagrelor in their body, the dosage of bentracimab was sufficient to reverse the effect of ticagrelor just as effectively in patients who last used ticagrelor between 24 to 48 hours, who were expected to have less ticagrelor in their body.

Table 10 shows the proportion achieving effective hemostasis (excellent or good) in surgery and major bleeding groups. These endpoints were met. The average proportion of patients achieving effective hemostasis was 95% in total enrolled (dosed) population, and 100% for patients requiring surgery or invasive procedure (surgery group). Surprisingly, 79.5% of patients with major bleeding were able to achieve effective hemostasis, including patients who received bentracimab during surgery, benefitting from the rapid action of bentracimab to reverse ticagrelor-enhanced bleeding.

TABLE 10
Proportion achieving effective hemostasis
in surgery and major bleeding groups

| | Eligible | | |
|---|---|---|---|
| | Total | Surgery | Major Bleeding |
| | (N = 180) | (N = 136) | (N = 44) |
| n (Proportion) | 171 | 136 | 35 |
| Proportion | 95.0% (91.8%, | 100% (100%, | 79.5% (67.6%, |
| (95% CI) | 98.2%) | 100%) | 91.5%) |
| P-value | <.0001 | <.0001 | <.0001 |

Table 11 shows a safety results summary of treatment emergent adverse events in the study. Treatment-emergent adverse events (TEAEs) are defined as adverse events with onset date/time after starting bentracimab infusion through 5 days after the end of infusion. Despite a high incidence of adverse events in the total study population (88.5% with at least 1 TEAE), only 1.6% of TEAEs were related to bentracimab. There were no deaths, serious adverse events, or severe adverse events related to bentracimab.

TABLE 11
Safety results summary: Treatment emergent adverse events

| | Safety (Dosed) | | Eligible Enrolled | | | |
|---|---|---|---|---|---|---|
| | Total | | Surgery | | Major Bleeding | |
| | (N = 191) | | (N = 136) | | (N = 44) | |
| At least 1 TEAE | 169 | (88.5%) | 122 | (89.7%) | 38 | (86.4%) |
| TEAEs related | 3 | (1.6%) | 2 | (1.5%) | 1 | (2.3%) |
| to bentracimab | | | | | | |
| Deaths | 3 | (1.6%) | 1 | (0.7%) | 2 | (4.6%) |
| Deaths related | 0 | | 0 | | 0 | |
| to bentracimab | | | | | | |
| SAEs | 28 | (14.7%) | 17 | (12.5%) | 9 | (20.5%) |
| SAEs related | 0 | | 0 | | 0 | |
| to bentracimab | | | | | | |
| Severe AEs | 37 | (19.4%) | 24 | (17.8%) | 11 | (25.0%) |

| Severe AEs related | 0 | | 0 | | 0 |
|---|---|---|---|---|---|
| to bentracimab | | | | | |
| Bentracimab | 1 | (0.5%) | 1 | (0.7%) | 0 |
| discontinuation | | | | | |
| due to TEAEs | | | | | |

Table 12 shows a safety results summary of post-enrollment adverse events. Post-enrollment adverse events (AEs) are defined as adverse events with onset date/time after starting bentracimab infusion through the end of study. Despite a high incidence of adverse events in the total study population (94.8% with at least 1 AE), only 2.1% of AEs were related to bentracimab. There were no deaths, serious adverse events (SAE), or severe adverse events related to bentracimab.

TABLE 12
Safety results summary: Post-enrollment adverse events

| | Safety (Dosed) | | Eligible Enrolled | | | |
|---|---|---|---|---|---|---|
| | Total | | Surgery | | Major Bleeding | |
| | (N = 191) | | (N = 136) | | (N = 44) | |
| At least 1 AE | 181 | (94.8%) | 127 | (93.4%) | 43 | (97.7%) |
| AEs related | 4 | (2.1%) | 3 | (2.1%) | 1 | (2.3%) |
| to bentracimab | | | | | | |
| Deaths | 8 | (4.2%) | 4 | (2.9%) | 4 | (9.1%) |
| Deaths related | 0 | | 0 | | 0 | |
| to bentracimab | | | | | | |
| SAEs | 61 | (31.9%) | 38 | (27.9%) | 21 | (47.7%) |
| SAEs related | 0 | | 0 | | 0 | |
| to bentracimab | | | | | | |
| Severe AEs | 54 | (28.3%) | 32 | (23.5%) | 20 | (45.5%) |

| Severe AEs related | 0 | | 0 | | 0 |
|---|---|---|---|---|---|
| to bentracimab | | | | | |
| Bentracimab | 1 | (0.5%) | 1 | (0.7%) | 0 |
| discontinuation | | | | | |
| due to TEAEs | | | | | |

Table 13 shows a safety results summary of treatment-emergent AEs (TEAEs) by preferred term in ≥10% of total enrolled subjects or surgery or major bleeding groups. Treatment-emergent AEs (TEAEs) are defined as AEs with onset date/time after starting bentracimab infusion through 5 days after the end of infusion.

TABLE 13
TEAEs by Preferred Term in ≥10% of Total Enrolled
Subjects or Surgery or Major Bleeding Groups

| | Enrolled | | Eligible Enrolled | | | |
|---|---|---|---|---|---|---|
| | Total | | Surgery | | Major Bleeding | |
| | (N = 191) | | (N = 136) | | (N = 44) | |
| Procedural pain | 41 | (21.5%) | 38 | (27.9%) | 3 | (6.8%) |
| Atrial fibrillation | 40 | (20.9%) | 30 | (22.1%) | 7 | (15.9%) |
| Hypotension | 34 | (17.8%) | 33 | (24.3%) | 0 | |
| Incision site pain | 35 | (18.3%) | 35 | (25.7%) | 0 | |
| Hypokalaemia | 24 | (12.6%) | 19 | (14.0%) | 5 | (11.4%) |
| Nausea | 25 | (13.1%) | 23 | (16.9%) | 2 | (4.5%) |
| Delirium | 25 | (13.1%) | 20 | (14.7%) | 5 | (11.4%) |
| Hypervolaemia | 26 | (13.6%) | 18 | (13.2%) | 7 | (15.9%) |
| Oedema peripheral | 23 | (12.0%) | 23 | (16.9%) | 0 | |
| Hypophosphataemia | 18 | (9.4%) | 12 | (8.8%) | 6 | (13.6%) |
| Hyperglycemia | 16 | (8.4%) | 16 | (11.8%) | 0 | |
| Pneumonia | 6 | (3.1%) | 1 | (0.7%) | 5 | (11.4%) |

Table 14 shows serious TEAEs by preferred term in >2.5% of total subjects or surgery or major bleeding groups. TEAEs are defined as AEs with onset date/time after starting bentracimab infusion through 5 days after the end of infusion.

TABLE 14
Serious TEAEs by Preferred Term in >2.5% of
Total Subjects or Surgery or Major Bleeding Groups

| | |
|---|---|
| Enrolled | Eligible |

| | Total | Surgery | Major Bleeding |
|---|---|---|---|
| | (N = 191) | (N = 136) | (N = 44) |
| Cardiac tamponade | 2 (1.0%) | 0 | 2 (4.5%) |

Tables 15-18 show pharmacokinetic data for total and free bentracimab (Tables 15-16) and total and free ticagrelor (Tables 17-18). Tables 15 and 16 shows total (Table 15) and free (Table 16) bentracimab levels in patients requiring surgery or invasive procedure (surgery group) and patients with major or life-threatening (major bleeding group). A high level of both total and free bentracimab is present in the blood 5-10 minutes after starting administration, i.e., when the patient is given the first segment (bolus) of infusion of bentracimab; followed by an intermediate level of bentracimab when the rate of infusion is reduced during 10 minutes to 4 hours after starting administration; followed by a lower level of bentracimab when the rate of infusion is further reduced during 4 hours to 24 hours after starting bentracimab. BLOQ refers to “below limit of quantification.” SEM refers to standard error of the sample. CV refers to coefficient of variation.

TABLE 15
Total Bentracimab Levels

| | | | |
|---|---|---|---|
| Subject | | | |
| Group | 5-10 | 30 | 1 |
| Statistic | mins | mins | hour |

Surgery or Procedure (N = 136)

| n | 136 | | 136 | | 136 | |
|---|---|---|---|---|---|---|
| n (BLOQ) | 0 | | 0 | | 0 | |
| Mean (SD) | 2497.50 | (4570.14) | 1844.79 | (752.02) | 1611.27 | (633.80) |
| SEM | 391.89 | | 64.49 | | 54.35 | |
| CV % | 182.99 | | 40.76 | | 39.34 | |
| Geometric | 1727.62 | (69.27) | 1738.79 | (34.24) | 1522.17 | (33.87) |
| mean of | | | | | | |
| CV % | | | | | | |
| Median | 1593.34 | | 1718.07 | | 1525.91 | |
| Q1, Q3 | 1278.09, | 2067.01 | 1415.92, | 2139.84 | 1236.73, | 1907.33 |
| Min, Max | 434.97, | 34311.73 | 518.27, | 7523.28 | 423.66, | 6431.87 |

Major Bleeding (N = 44)

| n | 43 | | 43 | | 43 | |
|---|---|---|---|---|---|---|
| n (BLOQ) | 0 | | 0 | | 0 | |
| Mean (SD) | 2874.12 | (8875.65) | 1815.06 | (469.25) | 1673.49 | (485.61) |
| SEM | 1353.52 | | 71.56 | | 74.05 | |
| CV % | 308.81 | | 25.85 | | 29.02 | |
| Geometric | 1521.34 | (83.25) | 1757.44 | (26.22) | 1603.80 | (30.74) |
| mean of | | | | | | |
| CV % | | | | | | |
| Median | 1558.00 | | 1772.33 | | 1654.59 | |
| Q1, Q3 | 1219.72, | 1852.20 | 1448.62, | 2086.49 | 1331.88, | 1961.62 |
| Min, Max | 446.6, | 59588.4 | 891.48, | 2911.34 | 722.62, | 2802.09 |

| Subject | | | | | | |
|---|---|---|---|---|---|---|
| Group | 4 | | 12 | | 24 | |
| Statistic | hours | | hours | | hours | |
| | Surgery or Procedure (N = 136) | | | | | |
| n | 136 | | 136 | | 111 | |
| n (BLOQ) | 0 | | 0 | | 1 | |
| Mean (SD) | 1519.45 | (2374.57) | 642.05 | (471.84) | 200.46 | (383.00) |
| SEM | 203.62 | | 40.46 | | 36.35 | |
| CV % | 156.28 | | 73.49 | | 191.06 | |
| Geometric | 1243.37 | (51.14) | 534.52 | (63.24) | 106.04 | (122.06) |
| mean of | | | | | | |
| CV % | | | | | | |
| Median | 1242.22 | | 515.88 | | 83.29 | |
| Q1, Q3 | 967.64, | 1487.29 | 373.33, | 710.87 | 53.79, | 162.13 |
| Min, Max | 339.99, | 27838.25 | 115, | 2877.51 | 0, | 3052.77 |
| | Major Bleeding (N = 44) | | | | | |
| n | 41 | | 42 | | 39 | |
| n (BLOQ) | 0 | | 0 | | 0 | |
| Mean (SD) | 1548.76 | (931.92) | 684.61 | (374.89) | 253.34 | (255.40) |
| SEM | 145.54 | | 57.85 | | 40.90 | |
| CV % | 60.17 | | 54.76 | | 100.81 | |
| Geometric | 1406.92 | (41.78) | 606.92 | (51.43) | 166.80 | (113.15) |
| mean of | | | | | | |
| CV % | | | | | | |
| Median | 1359.00 | | 586.63 | | 186.77 | |
| Q1, Q3 | 1111.90, | 1720.25 | 432.63, | 884.52 | 69.07, | 289.37 |
| Min, Max | 624.53, | 6625.53 | 231.25, | 1968.48 | 46.57, | 1039.15 |

TABLE 16
Free Bentracimab Levels

| | | | |
|---|---|---|---|
| Subject | | | |
| Group | 5-10 | 30 | 1 |
| Statistic | mins | mins | hour |

Surgery or Procedure (N = 136)

| n | 136 | | 136 | | 136 | |
|---|---|---|---|---|---|---|
| n (BLOQ) | 0 | | 0 | | 2 | |
| Mean (SD) | 1943.87 | (3792.17) | 1318.24 | (684.67) | 1064.33 | (569.51) |
| SEM | 325.18 | | 58.71 | | 48.84 | |
| CV % | 195.08 | | 51.94 | | 53.51 | |
| Geometric | 1250.05 | (85.55) | 1115.89 | (79.23) | 898.74 | (85.95) |
| mean of | | | | | | |
| CV % | | | | | | |
| Median | 1220.00 | | 1295.00 | | 1060.00 | |
| Q1, Q3 | 954.50, | 1680.00 | 910.50, | 1605.00 | 734.50, | 1375.00 |
| Min, Max | 132, | 27700 | 43, | 5140 | 0, | 3980 |

Major Bleeding (N = 44)

| n | 38 | | 38 | | 39 | |
|---|---|---|---|---|---|---|
| n (BLOQ) | 0 | | 1 | | 0 | |
| Mean (SD) | 2613.95 | (9764.32) | 1176.08 | (504.10) | 954.05 | (497.61) |
| SEM | 1583.98 | | 81.78 | | 79.68 | |
| CV % | 373.55 | | 42.86 | | 52.16 | |
| Geometric | 948.56 | (122.35) | 1096.01 | (52.92) | 722.58 | (132.07) |
| mean of | | | | | | |
| CV % | | | | | | |
| Median | 956.50 | | 1135.00 | | 1040.00 | |
| Q1, Q3 | 510.00, | 1430.00 | 947.00, | 1500.00 | 565.00, | 1290.00 |
| Min, Max | 227, | 61100 | 0, | 2300 | 25.9, | 2110 |

| Subject | | | | | | |
|---|---|---|---|---|---|---|
| Group | 4 | | 12 | | 24 | |
| Statistic | hours | | hours | | hours | |
| | Surgery or Procedure (N = 136) | | | | | |
| n | 136 | | 136 | | 111 | |
| n (BLOQ) | 0 | | 1 | | 69 | |
| Mean (SD) | 1031.38 | (1871.78) | 385.15 | (293.02) | 58.64 | (165.19) |
| SEM | 160.50 | | 25.13 | | 15.68 | |
| CV % | 181.48 | | 76.08 | | 281.69 | |
| Geometric | 775.07 | (71.93) | 315.42 | (70.36) | 78.98 | (146.36) |
| mean of | | | | | | |
| CV % | | | | | | |
| Median | 851.50 | | 288.50 | | 0 | |
| Q1, Q3 | 584.50, | 1050.00 | 218.00, | 452.50 | 0.00, | 37.90 |
| Min, Max | 82.6, | 21800 | 0, | 1900 | 0, | 1330 |
| | Major Bleeding (N = 44) | | | | | |
| n | 38 | | 36 | | 34 | |
| n (BLOQ) | 0 | | 1 | | 15 | |
| Mean (SD) | 929.01 | (554.12) | 394.81 | (243.72) | 80.07 | (120.65) |
| SEM | 89.89 | | 40.62 | | 20.69 | |
| CV % | 59.65 | | 61.73 | | 150.67 | |
| Geometric | 779.76 | (81.11) | 352.50 | (56.91) | 94.33 | (122.88) |
| mean of | | | | | | |
| CV % | | | | | | |
| Median | 937.00 | | 331.50 | | 27.30 | |
| Q1, Q3 | 685.00, | 1050.00 | 236.50, | 457.00 | 0.00, | 95.00 |
| Min, Max | 33.3, | 3610 | 0, | 1010 | 0, | 408 |

Tables 17 and 18 shows total (Table 17) and free (Table 18) ticagrelor levels in patients requiring surgery or invasive procedure (surgery group) and patients with major or life-threatening (major bleeding group). While there is detectable total ticagrelor 24 hours after starting administration of bentracimab, free ticagrelor is surprisingly depleted rapidly to below the limit of quantification (BLOQ) for most patients requiring surgery or invasive procedure (surgery group) and patients with major or life-threatening (major bleeding group) within 5-10 minutes after starting bentracimab and the effect is sustained over 24 hours.

TABLE 17
Total Ticagrelor Levels

| Subject | | | | |
|---|---|---|---|---|
| Group | | 5-10 | 30 | 1 |
| Statistic | Pre-dose | mins | mins | hour |

Surgery or Procedure (N = 136)

| n | 135 | | 136 | | 136 | | 136 | |
|---|---|---|---|---|---|---|---|---|
| n (BLOQ) | 0 | | 0 | | 0 | | 0 | |
| Mean (SD) | 343.55 | (397.46) | 1503.73 | (1696.70) | 2307.66 | (2549.85) | 2484.85 | (2688.15) |
| SEM | 34.21 | | 145.49 | | 218.65 | | 230.51 | |
| CV % | 115.69 | | 112.83 | | 110.50 | | 108.18 | |
| Geometric | 157.93 | (289.34) | 693.48 | (282.80) | 1003.89 | (329.86) | 1060.93 | (347.98) |
| mean of | | | | | | | | |
| CV % | | | | | | | | |
| Median | 213.00 | | 878.50 | | 1315.00 | | 1475.00 | |
| Q1, Q3 | 60.30, | 497.00 | 286.50, | 2245.00 | 362.00, | 3545.00 | 381.00, | 3730.00 |
| Min, Max | 2.88, | 2490 | 11.5, | 9640 | 13.9, | 11100 | 11.9, | 10200 |

Major Bleeding (N = 44)

| n | 44 | | 42 | | 42 | | 44 | |
|---|---|---|---|---|---|---|---|---|
| n (BLOQ) | 2 | | 1 | | 1 | | 1 | |
| Mean (SD) | 323.99 | (511.38) | 1423.06 | (1591.25) | 2484.93 | (2897.34) | 2606.08 | (3037.32) |
| SEM | 77.09 | | 245.54 | | 447.07 | | 457.89 | |
| CV % | 157.84 | | 111.82 | | 116.60 | | 116.55 | |
| Geometric | 122.83 | (322.21) | 604.01 | (374.11) | 1069.90 | (384.16) | 1115.93 | (372.80) |
| mean of | | | | | | | | |
| CV % | | | | | | | | |
| Median | 116.00 | | 704.50 | | 1230.00 | | 1275.00 | |
| Q1, Q3 | 32.60 | | 204.00, | 1940.00 | 367.00, | 3410.00 | 394.50, | 3765.00 |
| Min, Max | 0, | 2630 | 0, | 5160 | 0, | 12900 | 0, | 13600 |

| Subject | | | | | | |
|---|---|---|---|---|---|---|
| Group | 4 | | 12 | | 24 | |
| Statistic | hours | | hours | | hours | |
| | Surgery or Procedure (N = 136) | | | | | |
| n | 136 | | 136 | | 111 | |
| n (BLOQ) | 0 | | 0 | | 0 | |
| Mean (SD) | 1552.01 | (1768.82) | 520.17 | (782.19) | 341.67 | (510.48) |
| SEM | 151.68 | | 67.07 | | 48.45 | |
| CV % | 113.97 | | 150.37 | | 149.41 | |
| Geometric | 613.90 | (393.65) | 222.49 | (264.24) | 180.13 | (176.63) |
| mean of | | | | | | |
| CV % | | | | | | |
| Median | 829.50 | | 268.50 | | 197.00 | |
| Q1, Q3 | 206.00, | 2410.00 | 87.45, | 640.00 | 92.50, | 380.00 |
| Min, Max | 6.42, | 8470 | 4.41, | 5740 | 4.53, | 3620 |
| | Major Bleeding (N = 44) | | | | | |
| n | 42 | | 42 | | 39 | |
| n (BLOQ) | 1 | | 1 | | 1 | |
| Mean (SD) | 1970.18 | (2958.95) | 813.28 | (1291.09) | 540.22 | (746.35) |
| SEM | 456.58 | | 199.22 | | 119.51 | |
| CV % | 150.19 | | 158.75 | | 138.16 | |
| Geometric | 703.74 | (377.04) | 312.66 | (270.38) | 282.43 | (173.98) |
| mean of | | | | | | |
| CV % | | | | | | |
| Median | 726.50 | | 282.00 | | 222.00 | |
| Q1, Q3 | 214.00, | 2560.00 | 88.60, | 796.00 | 102.00, | 584.00 |
| Min, Max | 0, | 12800 | 0, | 5760 | 0, | 3440 |

TABLE 18
Free Ticagrelor Levels

| Subject | | | | |
|---|---|---|---|---|
| Group | | 5-10 | 30 | 1 |
| Statistic | Pre-dose | mins | mins | hour |

Surgery or Procedure (N = 136)

| n | 136 | | 136 | | 136 | | 136 | |
|---|---|---|---|---|---|---|---|---|
| n (BLOQ) | 27 | | 133 | | 132 | | 132 | |
| Mean (SD) | 0.294 | (0.3711) | 0.004 | (0.0284) | 0.002 | (0.0117) | 0.002 | (0.0147) |
| SEM | 0.03 | | 0 | | 0 | | 0 | |
| CV % | 126.11 | | 676.45 | | 664.73 | | 742.87 | |
| Geometric | 0.21 | (160.13) | 0.19 | (18.67) | 0.05 | (84.76) | 0.05 | (93.01) |
| mean of | | | | | | | | |
| CV % | | | | | | | | |
| Median | 0.15 | | 0 | | 0 | | 0 | |
| Q1, Q3 | 0.03, | 0.46 | 0, | 0 | 0, | 0 | 0, | 0 |
| Min, Max | 0, | 1.67 | 0, | 0.23 | 0, | 0.11 | 0, | 0.16 |

Major Bleeding (N = 44)

| n | 43 | | 42 | | 41 | | 43 | |
|---|---|---|---|---|---|---|---|---|
| n (BLOQ) | 4 | | 38 | | 38 | | 39 | |
| Mean (SD) | 0.435 | (0.6955) | 0.014 | (0.0645) | 0.027 | (0.1547) | 0.029 | (0.1235) |
| SEM | 0.11 | | 0.01 | | 0.02 | | 0.02 | |
| CV % | 159.93 | | 458.94 | | 581.79 | | 431.59 | |
| Geometric | 0.21 | (214.50) | 0.08 | (196.57) | 0.13 | (518.21) | 0.16 | (332.77) |
| mean of | | | | | | | | |
| CV % | | | | | | | | |
| Median | 0.17 | | 0 | | 0 | | 0 | |
| Q1, Q3 | 0.05, | 0.52 | 0, | 0 | 0, | 0 | 0, | 0 |
| Min, Max | 0, | 3.56 | 0, | 0.4 | 0, | 0.99 | 0, | 0.72 |

| Subject | | | | | | |
|---|---|---|---|---|---|---|
| Group | 4 | | 12 | | 24 | |
| Statistic | hours | | hours | | hours | |
| | Surgery or Procedure (N = 136) | | | | | |
| n | 136 | | 136 | | 111 | |
| n (BLOQ) | 131 | | 130 | | 87 | |
| Mean (SD) | 0.002 | (0.0111) | 0.003 | (0.0166) | 0.018 | (0.0560) |
| SEM | 0 | | 0 | | 0.01 | |
| CV % | 578.33 | | 580.28 | | 306.21 | |
| Geometric | 0.05 | (55.03) | 0.05 | (91.20) | 0.06 | (93.66) |
| mean of | | | | | | |
| CV % | | | | | | |
| Median | 0 | | 0 | | 0 | |
| Q1, Q3 | 0, | 0 | 0, | 0 | 0, | 0 |
| Min, Max | 0, | 0.1 | 0, | 0.14 | 0, | 0.46 |
| | Major Bleeding (N = 44) | | | | | |
| n | 42 | | 42 | | 39 | |
| n (BLOQ) | 39 | | 39 | | 36 | |
| Mean (SD) | 0.019 | (0.1126) | 0.005 | (0.0171) | 0.009 | (0.0358) |
| SEM | 0.02 | | 0 | | 0.01 | |
| CV % | 598.64 | | 378.81 | | 410.37 | |
| Geometric | 0.09 | (537.06) | 0.06 | (38.05) | 0.09 | (95.86) |
| mean of | | | | | | |
| CV % | | | | | | |
| Median | 0 | | 0 | | 0 | |
| Q1, Q3 | 0, | 0 | 0, | 0 | 0, | 0 |
| Min, Max | 0, | 0.73 | 0, | 0.08 | 0, | 0.2 |

### Example 2: Bentracimab Phase 3 Compared to PLATO Control Efficacy Results

This Example describes a comparison between the Bentracimab Phase 3 study and the PLATelet Inhibition and Patient Outcome Study (hereafter “PLATO study”). A subset of patients in the PLATO study was used as an external control for assessment of the efficacy and safety of bentracimab in its Phase 3 study (described in Example 1; hereafter “Phase 3 study”). The PLATO study was a multicenter, double-blind, randomized study investigating the use of ticagrelor for the prevention of cardiovascular events in 18,624 patients admitted to the hospital with an acute coronary syndrome.

PLATO reported measures of hemostasis in groups of patients having urgent surgery or major bleeding, fatal or life-threatening. Patients in whom urgent surgery or major bleeding occurred within 3 days of the last ticagrelor dose were identified. The first patient was enrolled in October 2006 and the last patient completed the study in February 2009.

Patients were randomly assigned to treatment with ticagrelor (n=9333) within 24 hours of onset of acute coronary syndrome. These patients received a 180-mg loading dose of ticagrelor followed by a maintenance dose of 90 mg of ticagrelor twice daily (BID). In patients undergoing CABG, ticagrelor was to be withheld for 24 to 72 hours before surgery. All patients received ASA at a dose of 75 to 100 mg daily unless they could not tolerate it. For those who had not previously been receiving aspirin, 325 mg was the preferred loading dose; 325 mg was also permitted as the daily dose for 6 months after stent placement.

Statistical analyses were performed to compare the efficacy and safety of bentracimab following ticagrelor in the Phase 3 study to that of ticagrelor without a reversal agent (external control) from the PLATO study to support the benefit: risk analyses of bentracimab.

### Protocol

### Objectives and Endpoints

PLATO study objectives relevant for comparisons to the Phase 3 study were:
To evaluate the occurrence of major and minor bleeding events
To evaluate all-cause mortality rates

A summary of the endpoints that were compared between Phase 3 bentracimab-treated patients and the external control patients from PLATO is presented in Table 19.

TABLE 19
Study Endpoints for Phase 3 and PLATO Control Comparisons

| Analysis Set | Endpoint | Description |
|---|---|---|
| Urgent Surgery | Primary Efficacy: | Proportion of patients with |
| Analysis Set | GUSTO severe or | GUSTO severe or life- |
| | life-threatening | threatening bleeding within 1 |
| | bleeding | day after start of urgent |
| | | surgery (i.e., do not achieve |
| | | effective hemostasis) |
| | Secondary Efficacy: | Proportion of patients who |
| | All-cause mortality | died within 38 days after |
| | | start of urgent surgery |
| | Safety: SAEs | Incidence of SAEs with |
| | | onset within 38 days after |
| | | start of urgent surgery |
| Major Bleeding | Secondary Efficacy: | Proportion of patients who |
| Analysis Set | All-cause mortality | died within 38 days of major |
| | | bleeding |
| | Safety: SAEs | Incidence of SAEs with |
| | | onset within 38 days after |
| | | start of major bleeding |
| Combined | Secondary Efficacy: | Proportion of patients who |
| Analysis Set | All-cause mortality | died within 38 days after |
| | | start of urgent surgery or |
| | | major bleeding |
| | Safety: SAEs | Incidence of SAEs with |
| | | onset within 38 days after |
| | | start of urgent surgery or |
| | | major bleeding |

To ensure comparability of the period over which efficacy and safety were evaluated in the two studies, Day 1 was defined in both studies as the day of urgent surgery for the urgent surgery group and the day of onset of major bleeding for the major bleeding group. Only data from PLATO through 38 days after the start of each patient's first identified urgent surgery or major bleeding event were included in the analyses to correspond to the 35±3 day follow-up in the Phase 3 study.

### Major Bleeding and GUSTO Bleeding Scale

The primary safety endpoint in the PLATO study was total major bleeding. The PLATO definitions of bleeding were chosen as inclusive and clinically relevant measures suitable for assessing bleeding events in the context of surgical (or other procedures) and medical treatment (Becker et al, European Heart Journal 32.23 (2011): 2933-2944). Bleeding not associated with a procedure was categorized as ‘spontaneous.’

PLATO major bleeding events were defined as:
Major life-threatening bleeding, which was fatal bleeding, intracranial bleeding, intrapericardial bleeding with cardiac tamponade, hypovolemic shock or severe hypotension due to bleeding and requiring pressors or surgery, a decline in the hemoglobin level of 5.0 g per deciliter or more, or the need for transfusion of at least 4 units of red cells
Other major bleeding, which was bleeding that led to clinically significant disability (e.g., intraocular bleeding with permanent vision loss) or bleeding either associated with a drop in the hemoglobin level of at least 3.0 g per deciliter but less than 5.0 g per deciliter or requiring transfusion of 2 to 3 units of red cells.
Secondary safety endpoints in the PLATO study included major bleeding assessed using the GUSTO bleeding scale. In PLATO, bleeding events were mapped onto the GUSTO bleeding scale by applying an algorithm (Becker et al, European heart journal 32.23 (2011): 2933-2944).

The GUSTO clinical bleeding scale was used to assess bleeding events for surgery patients in both Phase 3 and PLATO. See, GUSTO. (1993). N Engl J Med, 673-82.
GUSTO mild bleeding: does not meet criteria for either moderate or severe bleeding
GUSTO moderate bleeding: requires blood transfusion but does not result in hemodynamic compromise
GUSTO severe bleeding: fatal bleeding, intracranial hemorrhage, or bleeding that causes hemodynamic compromise and requires pharmacologic or surgical intervention

The proportion of urgent surgery patients who achieve effective hemostasis within 24 hours of surgery is the adjudicated first secondary efficacy endpoint in the Phase 3 study. Effectiveness of hemostasis is assessed using the GUSTO criteria. GUSTO severe or life-threatening bleeding (hereafter called GUSTO severe bleeding) is classified as ineffective hemostasis.

Categories of GUSTO severe bleeding in the PLATO study were: major bleeding, fatal or life-threatening; major bleeding, other; and minor bleeding. The definitions of bleeding events in the PLATO study were:
Major bleed—fatal/life-threatening. Any one of the following: 1) fatal; 2) intracranial; 3) intrapericardial bleed with cardiac tamponade; 4) hypovolemic shock or severe hypotension due to bleeding and requiring pressors or surgery; 5) clinically overt or apparent bleeding associated with a decrease in Hb of more than 50 g/L (3.1 mmol/L; 0.775 mmol/L); or 6) transfusion of 4 or more units (whole blood or packed red blood cells (PRBCs)) for bleeding.
Major bleed—other. Any one of the following: 1) Significantly disabling (e.g., intraocular with permanent vision loss); 2) Clinically overt or apparent bleeding associated with a decrease in Hb of 30 g/L (1.9 mmol/L; 0.465 mmol/L) to 50 g/L (3.1 mmol/L; 0.775 mmol/L); or 3) transfusion of 2-3 units (whole blood or PRBCs) for bleeding.
Minor bleed—requires medical intervention to stop or treat bleeding (e.g., epistaxis requiring visit to medical facility for packing).
Minimal bleed—all others (e.g., bruising, bleeding gums, oozing from injection sites, etc.) not requiring intervention or treatment.

A comparison of the proportions of patients in the Phase 3 and PLATO studies with GUSTO severe or life-threatening bleeding within 24 hours after start of surgery (or 1 calendar day, if bleed and/or surgery times not available) provided a clinically meaningful assessment of the clinical benefit of bentracimab.

### All-Cause Mortality

Deaths of all causes were collected in both the Phase 3 and PLATO studies. This is a clinically meaningful endpoint that is applicable to both the urgent surgery and major bleeding group and was evaluated in each group separately and combined. The proportion of patients who died due to any cause through Day 38 was compared between Phase 3 and PLATO patients.

### Serious Adverse Events

SAEs reflect safety of bentracimab in patients who recently received ticagrelor and contribute to the risk profile of bentracimab treatment. SAEs were defined consistently in the Phase 3 and PLATO protocols. SAE incidences through Day 38 were compared between Phase 3 and PLATO patients.

### Handling of Missing/Incomplete Values

For assessment of proportion of patients with severe bleeding events within 24 hours after the start of urgent surgery, only patients with adjudicated bleeding assessments were included. Sensitivity analyses assessing the impact of excluding patients that had urgent surgery, but did not have bleeding assessments.

For assessment of all-cause mortality, only patients who were not lost to follow-up between Day 1 and Day 38 were included (i.e., complete case analysis), provided there were less than 5% of patients with missing mortality data in each of the treatment groups.

For the Major Bleeding Analysis Set, for which all-cause mortality were inferentially compared between treatment groups, if greater than 5% of patients in either treatment group had missing mortality data, then multiple imputation (MI) was used to impute the missing data. First, missing mortality data in each treatment group were imputed using the FCS LOGISTIC option in SAS® PROC MI, which imputes binary endpoints. Baseline covariates in the logistic model included those listed in Table 4 PLATO SAP for the Major Bleeding Analysis Set. This step was repeated to generate 30 imputed datasets. Next, the analysis model described below in Section 5.9.2 for all-cause mortality was performed on each of the 30 “complete” datasets (observed values plus imputed values). Finally, the estimated treatment effects were combined across the 30 analyses using SAS® PROC MIANALYZE.

### Patient Population

Patients were male and female, aged 18 years and over, with a non-ST or ST segment elevation ACS (index event) and with high risk of secondary thrombotic events.

All patients in the Phase 3 study who granted informed consent, met enrollment criteria for the urgent surgery group or major bleeding group (see, Example 1, Inclusion/Exclusion Criteria), and were dosed with bentracimab were included in the external control comparisons.

Only data from PLATO study patients who received ticagrelor were included. Further, all data from patients who withdrew their informed consent at any time during PLATO and all data from China, which did not allow data to be re-used based on informed consent wording, were excluded from the transfer.

The external control group from PLATO was chosen such that its characteristics are consistent with the inclusion criteria of the Phase 3 study (see, Example 1, Inclusion Criteria). Specifically, PLATO patients were selected based on the following criteria:

All patients in the Phase 3 study received ticagrelor within 3 days of urgent surgery or onset of major bleeding. Only patients in PLATO who received ticagrelor within 3 days of urgent surgery or onset of major bleeding were included in comparisons with patients in the Phase 3 study.

Only patients who had urgent surgery/invasive procedures are included in the Phase 3 urgent surgery group. Only patients in PLATO who had urgent surgery/procedures were included in comparisons with the urgent surgery patients in the Phase 3 study. For the PLATO urgent surgery group, only patients in PLATO who had urgent surgery/procedures and independent adjudication of surgery-related bleeding events were included for consistency with the independent adjudication of bleeding events in the Phase 3 study.

Aspirin use was allowed in both the Phase 3 and PLATO studies. However, daily doses of ASA >100 mg are associated with higher risk of bleeding (Altman, et al. Circulation 94.11 (1996): 3002-3003 and Patrono et al, New England Journal of Medicine 353.22 (2005): 2373-2383). In the Phase 3 study, in the data cut for the second interim analysis, all except 3 patients received a median daily dose of ASA ≤100 mg within the 7 days prior to urgent surgery or major bleeding. In the PLATO study, many patients had ASA doses of >100 mg per day. To reduce the confounding effects of aspirin use when comparing Phase 3 and PLATO outcomes, only patients in PLATO who received a median daily dose of ASA ≤100 mg within the 7 days prior to urgent surgery or major bleeding were included. Given the small number of Phase 3 patients with higher aspirin doses and to retain all the Phase 3 patients in the external control study, no Phase 3 patients were excluded based on higher ASA use.

Table 20 summarizes the patient disposition and analysis sets in the Phase 3 bentracimab and PLATO study. Among 191 total Phase 3 bentracimab patients, 136/191 (77%) of patients were assigned to urgent surgery analysis set and 41/191 (23%) to major bleeding analysis set, compared to 376/8973 (4.2%) assigned to urgent surgery analysis set and 175/8973 (2.0%) to major bleeding analysis set in the PLATO control. The urgent surgery analysis set represented 136/177 (76.8%) of the combined analysis set in bentracimab Phase 3 study and 240/374 (64.2%) in PLATO control.

TABLE 20
Patient Disposition and Analysis Sets

| | Phase 3 Bentracimab | | PLATO Control | |
|---|---|---|---|---|
| | (N = 191) | | (N = 8782) | |
| | n (%) | | n (%) | |
| Enrolled Analysis Set | 191 | (100.0) | 8782 | (100.0) |
| Urgent Surgery Analysis Set | 136 | (71.2) | 240 | (2.7) |
| Major Bleeding Analysis Set | 41 | (21.5) | 134 | (1.5) |
| Combined Analysis Set | 177 | (92.7) | 374 | (4.3) |

Demographics and baseline characteristics of the urgent surgery, major bleeding, and combined analysis sets of bentracimab and PLATO control patients are presented by analysis set in Table 21. Overall, disease characteristics indicated the bentracimab patients, in the urgent surgery analysis set or major bleeding analysis set or both, had more comorbidities (hypertension, prior MI, prior CABG, moderate renal dysfunction, anemia) than patients in the PLATO control. A higher proportion of bentracimab patients had 0 days off ticagrelor prior to surgery or major bleeding. Bentracimab patients were more likely to be female, have CABG surgery, have ICH, and be from North America and less likely to be White.

TABLE 21
Demographics and Baseline Characteristics by Analysis Set (Urgent
Surgery, Major Bleeding, and Combined Analysis Sets)

| | Urgent Surgery | | | | Major Bleeding | |
|---|---|---|---|---|---|---|
| Characteristic | Phase 3 | | PLATO Control | | Phase 3 | |
| Statistic | (N = 136) | | (N = 240) | | (N = 41) | |
| Age group, n (%) | | | | | | |
| ≤65 years | 62 | (45.6) | 120 | (50.0) | 20 | (48.8) |
| >65 years | 74 | (54.4) | 120 | (50.0) | 21 | (51.2) |
| ≤75 years | 107 | (78.7) | 195 | (81.3) | 28 | (68.3) |
| >75 years | 29 | (21.3) | 45 | (18.8) | 13 | (31.7) |
| Sex, n (%) | | | | | | |
| Female | 28 | (20.6) | 76 | (31.7) | 11 | (26.8) |
| Male | 108 | (79.4) | 164 | (68.3) | 30 | (73.2) |
| Race, n (%) | | | | | | |
| White | 114 | (83.8) | 237 | (98.8) | 37 | (90.2) |
| Black | 4 | (2.9) | 2 | (0.8) | 2 | (4.9) |
| Asian | 16 | (11.8) | 1 | (0.4) | 0 | |
| Other | 2 | (1.5) | 0 | | 2 | (4.9) |
| Body Mass Index (BMI) | | | | | | |
| ≤25 kg/m\hat{ }2 | 30 | (22.2) | 75 | (31.5) | 10 | (28.6) |
| >25 kg/m\hat{ }2 | 105 | (77.8) | 163 | (68.5) | 25 | (71.4) |
| Missing | 1 | | 2 | | 6 | |
| Imputed Baseline BMI | | | | | | |
| Group, n (%) | | | | | | |
| ≤25 kg/m\hat{ }2 | 30 | (22.1) | 76 | (31.7) | n/a | |
| >25 kg/m\hat{ }2 | 106 | (77.9) | 164 | (68.3) | n/a | |
| Missing | | | | | | |
| Region, n (%) | | | | | | |
| North America | 117 | (86.0) | 28 | (11.7) | 30 | (73.2) |
| Europe, Middle East, | 19 | (14.0) | 212 | (88.3) | 11 | (26.8) |
| and Africa | | | | | | |
| Diabetes, n (%) | | | | | | |
| Yes | 58 | (42.6) | 52 | (21.7) | 18 | (43.9) |
| No | 78 | (57.4) | 188 | (78.3) | 23 | (56.1) |
| Hypertension, n (%) | | | | | | |
| Yes | 108 | (79.4) | 149 | (62.1) | 31 | (75.6) |
| No | 28 | (20.6) | 91 | (37.9) | 10 | (24.4) |
| Prior MI, n (%) | | | | | | |
| Yes | 117 | (86.0) | 38 | (15.8) | 26 | (63.4) |
| No | 19 | (14.0) | 202 | (84.2) | 15 | (36.6) |
| Prior CABG or | | | | | | |
| Percutaneous Coronary | | | | | | |
| Artery Intervention, | | | | | | |
| n (%) | | | | | | |
| Yes | 26 | (19.1) | 20 | (8.3) | 31 | (75.6) |
| No | 110 | (80.9) | 220 | (91.7) | 10 | (24.4) |
| Prior Peripheral Arterial | | | | | | |
| Disease, n (%) | | | | | | |
| Yes | 2 | (1.5) | 16 | (6.7) | 2 | (4.9) |
| No | 134 | (98.5) | 224 | (93.3) | 39 | (95.1) |
| Baseline eGFR (mL/min) | | | | | | |
| n | 135 | | 231 | | 41 | |
| Mean (SD) | 83.675 | (33.1747) | 79.670 | (25.5061) | 77.883 | (40.8581) |
| Median | 82.311 | | 78.927 | | 70.790 | |
| Min, Max | 6.59, | 257.81 | 17.87, | 188.83 | 13.77, | 244.64 |
| Baseline eGFR | | | | | | |
| Category, n (%) | | | | | | |
| ≥90 mL/min (No renal | 48 | (35.6) | 68 | (29.4) | 14 | (34.1) |
| disease) | | | | | | |
| ≥60 and ≤89 mL/min | 63 | (46.7) | 116 | (50.2) | 10 | (24.4) |
| (Mild renal disease) | | | | | | |
| ≥30 and ≤859 mL/min | 18 | (13.3 | 41 | (17.7) | 16 | (39.0) |
| (Moderate renal disease) | | | | | | |
| ≥15 and ≤29 mL/min | 2 | (1.5) | 6 | (2.6) | 0 | |
| (Severe renal disease) | | | | | | |
| <15 mL/min (End | 4 | (3.0) | 0 | | 1 | (2.4) |
| stage renal disease) | | | | | | |
| Missing | 1 | | 9 | | 0 | |
| Imputed Baseline eGFR | | | | | | |
| Group, n (%) [c] | | | | | | |
| <60 mL/min | 24 | (17.6) | 52 | (21.7) | n/a | |
| ≥60 mL/min | 112 | (82.4) | 188 | (78.3) | n/a | |
| Missing | | | | | n/a | |
| Baseline Hemoglobin | | | | | | |
| (g/dL) | | | | | | |
| n | 129 | | 225 | | 38 | |
| Mean (SD) | 13.25 | (2.095) | 13.63 | (1.543) | 10.22 | (2.304) |
| Median | 13.60 | | 13.70 | | 9.75 | |
| Min, Max | 6.5, | 16.9 | 6.7, | 16.9 | 6.4, | 17.3 |
| Baseline Hemoglobin | | | | | | |
| Group, n (%) | | | | | | |
| ≤11 g/dL | 18 | (14.0) | 10 | (4.4) | 28 | (73.7) |
| >11 g/dL | 111 | (86.0) | 215 | (95.6) | 10 | (26.3) |
| Missing | 7 | | 15 | | 3 | |
| Imputed Baseline | | | | | | |
| Hemoglobin Group, | | | | | | |
| n (%) [c] | | | | | | |
| <=11 g/dL | 18 | (13.2) | 10 | (4.2) | n/a | |
| >11 g/dL | 118 | (86.8) | 230 | (95.8) | n/a | |
| Missing | | | | | n/a | |
| Type of Surgery, n (%) | | | | | | |
| CABG | 111 | (81.6) | 102 | (42.5) | n/a | |
| Non-CABG | 25 | (18.4) | 138 | (57.5) | n/a | |
| Days off Ticagrelor | | | | | | |
| before Surgery, n (%) | | | | | | |
| 0 | 105 | (77.2) | 193 | (80.4) | 33 | (80.5) |
| 1 | 23 | (16.9) | 29 | (12.1) | 8 | (19.5) |
| 2 | 8 | (5.9) | 18 | (7.5) | 0 | |
| Aspirin Use 7 Days Prior | | | | | | |
| to Surgery or Major | | | | | | |
| Bleeding, n (%) | | | | | | |
| 0 mg | 55 | (40.4) | 143 | (59.6) | 16 | (39.0) |
| >0 mg | 81 | (59.6) | 97 | (40.4) | 25 | (61.0) |
| Type of Major Bleed, | | | | | | |
| Fatal or Life- | | | | | | |
| Threatening, n (%) | | | | | | |
| Procedure-related | n/a | | n/a | | 16 | (39.0) |
| Spontaneous ICH | n/a | | n/a | | 15 | (36.6) |
| Spontaneous non-ICH | n/a | | n/a | | 10 | (24.4) |
| | Major Bleeding | | Combined | | | |
| Characteristic | PLATO Control | | Phase 3 | | PLATO Control | |
| Statistic | (N = 134) | | (N = 177) | | (N = 374) | |
| Age group, n (%) | | | | | | |
| ≤65 years | 56 | (41.8) | 82 | (46.3) | 176 | (47.1) |
| >65 years | 78 | (58.2) | 95 | (53.7) | 198 | (52.9) |
| ≤75 years | 104 | (77.6) | 135 | (76.3) | 299 | (79.9) |
| >75 years | 30 | (22.4) | 42 | (23.7) | 75 | (20.1) |
| Sex, n (%) | | | | | | |
| Female | 32 | (23.9) | 39 | (22.0) | 108 | (28.9) |
| Male | 102 | (76.1) | 138 | (78.0) | 266 | (71.1) |
| Race, n (%) | | | | | | |
| White | 133 | (99.3) | 151 | (85.3) | 370 | (98.9) |
| Black | 1 | (0.7) | 6 | (3.4) | 3 | (0.8) |
| Asian | 0 | | 16 | (9.0) | 1 | (0.3) |
| Other | 0 | | 4 | (2.3) | 0 | |
| Body Mass Index (BMI) | | | | | | |
| ≤25 kg/m\hat{ }2 | 32 | (23.9) | 40 | (23.5) | 107 | (28.8) |
| >25 kg/m\hat{ }2 | 102 | (76.1) | 130 | (76.5) | 265 | (71.2) |
| Missing | 0 | | 7 | | 2 | |
| Imputed Baseline BMI | | | | | | |
| Group, n (%) | | | | | | |
| ≤25 kg/m\hat{ }2 | n/a | | 40 | (23.4) | 108 | (28.9) |
| >25 kg/m\hat{ }2 | n/a | | 131 | (76.6) | 266 | (71.1) |
| Missing | | | 6 | | 0 | |
| Region, n (%) | | | | | | |
| North America | 11 | (8.2) | 147 | (83.1) | 39 | (10.4) |
| Europe, Middle East, | 123 | (91.8) | 30 | (16.9) | 335 | (89.6) |
| and Africa | | | | | | |
| Diabetes, n (%) | | | | | | |
| Yes | 38 | (28.4) | 76 | (42.9) | 90 | (24.1) |
| No | 96 | (71.6) | 101 | (57.1) | 284 | (75.9) |
| Hypertension, n (%) | | | | | | |
| Yes | 92 | (68.7) | 139 | (78.5) | 241 | (64.4) |
| No | 42 | (31.3) | 38 | (21.5) | 133 | (35.6) |
| Prior MI, n (%) | | | | | | |
| Yes | 32 | (23.9) | 143 | (80.8) | 70 | (18.7) |
| No | 102 | (76.1) | 34 | (19.2) | 304 | (81.3) |
| Prior CABG or | | | | | | |
| Percutaneous Coronary | | | | | | |
| Artery Intervention, | | | | | | |
| n (%) | | | | | | |
| Yes | 25 | (18.7) | 57 | (32.2) | 45 | (12.0) |
| No | 109 | (81.3) | 120 | (67.8) | 329 | (88.0) |
| Prior Peripheral Arterial | | | | | | |
| Disease, n (%) | | | | | | |
| Yes | 15 | (11.2) | 4 | (2.3) | 31 | (8.3) |
| No | 119 | (88.8) | 173 | (97.7) | 343 | (91.7) |
| Baseline eGFR (mL/min) | | | | | | |
| n | 132 | | 176 | | 363 | |
| Mean (SD) | 75.685 | (25.5948) | 82.325 | (35.0759) | 78.221 | (25.5752) |
| Median | 76.283 | | 79.820 | | 77.621 | |
| Min, Max | 14.14, | 176.15 | 6.59, | 257.81 | 14.14, | 188.83 |
| Baseline eGFR | | | | | | |
| Category, n (%) | | | | | | |
| ≥90 mL/min (No renal | 27 | (20.5) | 62 | (35.2) | 95 | (26.2) |
| disease) | | | | | | |
| ≥60 and ≤89 mL/min | 73 | (55.3) | 73 | (41.5) | 189 | (52.1) |
| (Mild renal disease) | | | | | | |
| ≥30 and ≤859 mL/min | 26 | (19.7) | 34 | (19.3) | 67 | (18.5) |
| (Moderate renal disease) | | | | | | |
| ≥15 and ≤29 mL/min | 5 | (3.8) | 2 | (1.1) | 11 | (3.0) |
| (Severe renal disease) | | | | | | |
| <15 mL/min (End | 1 | (0.8) | 5 | (2.8) | 1 | (0.3) |
| stage renal disease) | | | | | | |
| Missing | 2 | | 1 | | 11 | |
| Imputed Baseline eGFR | | | | | | |
| Group, n (%) [c] | | | | | | |
| <60 mL/min | n/a | | 41 | (23.2) | 84 | (22.6) |
| ≥60 mL/min | n/a | | 136 | (76.8) | 288 | (77.4) |
| Missing | n/a | | 0 | | 2 | |
| Baseline Hemoglobin | | | | | | |
| (g/dL) | | | | | | |
| n | 132 | | 167 | | 357 | |
| Mean (SD) | 13.51 | (2.011) | 12.56 | (2.489) | 13.58 | (1.729) |
| Median | 13.80 | | 13.20 | | 13.70 | |
| Min, Max | 7.4, | 18.7 | 6.4, | 17.3 | 6.7, | 18.7 |
| Baseline Hemoglobin | | | | | | |
| Group, n (%) | | | | | | |
| ≤11 g/dL | 14 | (10.6) | 46 | (27.5) | 24 | (6.7) |
| >11 g/dL | 118 | (89.4) | 121 | (72.5) | 333 | (93.3) |
| Missing | 2 | | 10 | | 17 | |
| Imputed Baseline | | | | | | |
| Hemoglobin Group, | | | | | | |
| n (%) [c] | | | | | | |
| <=11 g/dL | n/a | | 46 | (26.4) | 24 | (6.5) |
| >11 g/dL | n/a | | 128 | (73.6) | 348 | (93.5) |
| Missing | n/a | | 3 | | 2 | |
| Type of Surgery, n (%) | | | | | | |
| CABG | n/a | | 111 | (81.6) | 102 | (42.5) |
| Non-CABG | n/a | | 25 | (18.4) | 138 | (57.5) |
| Days off Ticagrelor | | | | | | |
| before Surgery, n (%) | | | | | | |
| 0 | 90 | (67.2) | 138 | (78.0) | 283 | (75.7) |
| 1 | 17 | (12.7) | 31 | (17.5) | 46 | (12.3) |
| 2 | 27 | (20.1) | 8 | (4.5) | 45 | (12.0) |
| Aspirin Use 7 Days Prior | | | | | | |
| to Surgery or Major | | | | | | |
| Bleeding, n (%) | | | | | | |
| 0 mg | 26 | (19.4) | 71 | (40.1) | 169 | (45.2) |
| >0 mg | 108 | (80.6) | 106 | (59.9) | 205 | (54.8) |
| Type of Major Bleed, | | | | | | |
| Fatal or Life- | | | | | | |
| Threatening, n (%) | | | | | | |
| Procedure-related | 82 | (61.2) | 16 | (39.0) | 82 | (61.2) |
| Spontaneous ICH | 14 | (10.4) | 15 | (36.6) | 14 | (10.4) |
| Spontaneous non-ICH | 38 | (28.4) | 10 | (24.4) | 38 | (28.4) |

### Results

### Primary Efficacy Endpoint: Severe GUSTO Bleeding (Urgent Surgery Analysis Set)

None of 136(0.0%) bentracimab urgent surgery patients had severe GUSTO bleeding, compared to 51/240 (21.3%) in the PLATO control (Table 22). A stabilized Inverse Probability of Treatment Weighting (sIPTW) propensity score weighting adjusted estimated risk of severe bleeding was 0.4 (95% CI: 0.0, 6.3) for bentracimab and 23.8 (95% CI: 18.7, 29.8) for PLATO control. The risk difference (bentracimab −control) of −23.4 (95% CI: (−29.0, −17.7)) indicates a statistically significant reduction in severe GUSTO bleeding in the bentracimab urgent surgery patients, P<0.0001. This result demonstrates that bentracimab treatment is associated with a statistically significantly lower rate of severe GUSTO bleeding than occurs in PLATO controls.

TABLE 22
Primary Analysis of Proportion of Patients with
GUSTO Severe Bleeding using sIPTW Propensity
Score Weighting (Urgent Surgery Analysis Set)

| | Phase 3 | | PLATO | |
|---|---|---|---|---|
| | Bentracimab | | Control | |
| | (N = 136) | | (N = 240) | |
| Had GUSTO Severe Bleeding, n (%) | | | | |
| Yes | 0 | (0.0) | 51 | (21.3) |
| No | 136 | (100.0) | 189 | (78.8) |
| Estimated Risk of Severe Bleeding | 0.4 | | 23.8 | |
| [a] | | | | |
| 95% CI | (0.0, | 6.3) | (18.7, | 29.8) |
| Risk Difference | −23.4 | | | |
| (Bentracimab − Control) | | | | |
| 95% CI | (−29.0, | −17.7) | | |

| | |
|---|---|
| P-value | <0.0001 |

Abbreviations: CI = Confidence Interval; sIPTW = Stabilized Inverse Probability of Treatment Weighting.
Note:
Refer to SAP section 4.4.1 for GUSTO severe bleeding definition and assessment in both studies.
[a] Risk and risk difference are estimated by weighted logistic regression model using Firth's penalized likelihood with sIPTW propensity score weights. The model predicts risk of GUSTO severe bleeding within 24-hours from the start of surgery with treatment group and type of surgery (CABG or non-CABG) as explanatory variables.

### Sensitivity Analyses of Primary Efficacy Endpoint (Urgent Surgery Analysis Set)

Sensitivity analyses of the primary endpoint using AIPW and ATT propensity score weighting adjustments are given in Table 23. In both analyses, risk differences (bentracimab−control) were statistically significantly different between bentracimab and PLATO controls, P<0.0001. For the AIPW analysis, risk difference was −24.0 (95% CI: −33.9, −14.0). For the ATT analysis, risk difference was −40.0 (95% CI: −52.5, −27.5). These sensitivity analyses support the primary efficacy endpoint showing that bentracimab treatment is associated with a statistically significantly lower rate of severe GUSTO bleeding than occurs in PLATO controls.

TABLE 23
Sensitivity Analysis of Proportion of Patients with GUSTO Severe Bleeding
using AIPW and ATT Propensity Score Weighting (Urgent Surgery Analysis Set)

| | AIPW Propensity Score | | | | ATT Propensity Score | | | |
|---|---|---|---|---|---|---|---|---|
| | Phase 3 Bentracimab | | PLATO Control | | Phase 3 Bentracimab | | PLATO Control | |
| | (N = 136) | | (N = 240) | | (N = 136) | | (N = 240) | |
| Had GUSTO Severe Bleeding, n (%) | | | | | | | | |
| Yes | 0 | (0.0) | 51 | (21.3) | 0 | (0.0) | 51 | (21.3) |
| No | 136 | (100.0) | 189 | (78.8) | 136 | (100.0) | 189 | (78.8) |
| Estimated Risk of Severe Bleeding [a, b] | 0.0 | | 24.0 | | | | | |
| 95% CI | (−0.0, | 0.0) | (14.0, 33.9) | | 0.6 | | 40.6 | |
| Risk Difference (Bentracimab − Control) | −24.0 | | | | −40.0 | | | |
| 95% CI | (−33.9, | −14.0) | | | (−52.5, | −27.5) | | |

| | | |
|---|---|---|
| P-value | <0.0001 | <0.0001 |

Abbreviations: AIPW = Augmented Inverse Probability Weighting; ATT = Average Treatment Effect of Treated; CI = Confidence Interval.
[a] Risk and risk difference are estimated by using augmented inverse probability weights (AIPW). In addition to a propensity score model, the AIPW estimation method incorporates a model for the outcome variable, GUSTO severe bleeding. The AIPW estimation method is doubly robust and provides unbiased estimates for the Average Treatment Effect (ATE) even if one of the outcome or propensity score models is mis-specified. The propensity score model includes effects for type of surgery (CABG or non-CABG), days off ticagrelor prior to surgery (0, 1, or 2), median aspirin dose 7 days prior to surgery (0 mg or >0 mg), sex (male or female), body mass index (<=25 or >25 kg/m²), eGFR (<60 or >=60 mL/min/1.73 m 2), hemoglobin (<=11 or >11 g/dL), age (<= 65 or >65), and region (North America or Other - includes Europe, Middle East, and Africa). The outcome model includes an effect for type of surgery (CABG or non-CABG).
[b] Risk and risk difference are estimated by weighted logistic regression model using Firth's penalized likelihood with ATT propensity score weights. The model predicts risk of GUSTO severe bleeding within 24-hours from the start of surgery with treatment group, type of surgery (CABG or non-CABG), and hemoglobin category (<=11 or >11 g/dL) as explanatory variables.

### Subset Analyses of Primary Efficacy Endpoint

Analyses were performed of the proportion of subjects who had severe GUSTO bleeding in various subsets of the urgent surgery population, based on type of urgent surgery (CABG, non-CABG), age (≤65, >65 years of age), gender (female, male), race (Asian, Black, White, Other), and region (North America, Europe, Middle East, and Africa) (Table 24).

No bentracimab patients had GUSTO severe bleeding.

In the PLATO control, 23/102 (22.5%) patients with CABG and 28/135 (20.3%) patients with non-CABG surgeries had severe GUSTO bleeding. The treatment differences and 95% CIs for differences in proportions between bentracimab and PLATO control were −22.5, 95% CI: −31.6, −14.8 for CABG patients and −20.3, 95% CI: −27.8, −5.7 for non-CABG patients. These subset analyses show benefit of bentracimab in reducing severe GUSTO bleeding without regard to type of surgery the patient has undergone and support the primary efficacy endpoint.

In the PLATO control, 21/120 (17.5%) patients ≤65 years of age and 30/120 (25.0%) patients >65 years of age had severe GUSTO bleeding. The treatment differences and 95% CIs for differences in proportions between bentracimab and PLATO control were −17.5, 95% CI: −25.3, −9.3 for patients ≤65 years of age and −25.0, 95% CI: −33.4, −16.5 for patients >65 years of age. These subset analyses show benefit of bentracimab in reducing severe GUSTO bleeding without regard to patient age and support the primary efficacy endpoint.

In the PLATO control, 50/237 (21.1%) White patients had severe GUSTO bleeding. The treatment difference and 95% CIs for difference in proportion between White bentracimab and PLATO control patients −21.1, 95% CI: −26.7, −15.4. This analysis in the largest patient subset by race indicates benefit of bentracimab and supports the primary efficacy endpoint. Sample sizes of other racial subsets were too small in either the bentracimab or PLATO control patients or both to draw any conclusion about treatment differences. Data on Asians are included in Table 24 below. Without regard for race, there were no instances of severe GUSTO bleeding in any urgent surgery patient treated with bentracimab.

In the PLATO control, 7/28 (25.0%) of patients from North America and 44/120 (20.8%) of patients from Europe, Middle East, and Africa had severe GUSTO bleeding. The treatment differences and 95% CIS for differences in proportions between bentracimab and PLATO control were −25.0, 95% CI: −43.4, −12.3 for North America and −20.8, 95% CI: −26.7, −3.2 for Europe, Middle East, and Africa. These subset analyses show benefit of bentracimab in reducing severe GUSTO bleeding without regard to region and support the primary efficacy endpoint.

TABLE 24
Subset Analyses of Proportion of Patients with GUSTO Severe Bleeding (Urgent Surgery Analysis Set)

| | Phase 3 Bentracimab | | PLATO Control | | Phase 3 Bentracimab | | PLATO Control | |
|---|---|---|---|---|---|---|---|---|
| | (N = 136) | | (N = 240) | | (N = 136) | | (N = 240) | |
| Urgent Surgery Subgroup | CABG | | | | Non-CABG | | | |
| | N = 111 | | N = 102 | | N = 25 | | N = 138 | |
| Had GUSTO Severe Bleeding [a], n (%) | | | | | | | | |
| Yes | 0 | (0.0) | 23 | (22.5) | 0 | (0.0) | 28 | (20.3) |
| No | 111 | (100.0) | 79 | (77.5) | 25 | (100.0) | 110 | (79.7) |
| 95% Wilson Score CI for Proportion with | (0.0, | 3.3) | (15.5, | 31.6) | (0.0, | 13.3) | (14.4, | 27.8) |
| GUSTO Severe Bleeding | | | | | | | | |
| Difference of Proportions (Bentracimab − | −22.5 | | | | −20.3 | | | |
| Control) | | | | | | | | |
| 95% Newcombe CI for Difference of | (−31.6, | −14.8) | | | (−27.8, | −5.7) | | |
| Proportions | | | | | | | | |
| Age | ≤65 years | | | | >65 years | | | |
| | N = 62 | | N = 120 | | N = 74 | | N = 120 | |
| Had GUSTO Severe Bleeding [a], n (%) | | | | | | | | |
| Yes | 0 | (0.0) | 21 | (17.5) | 0 | (0.0) | 30 | (25.0) |
| No | 62 | (100.0) | 99 | (82.5) | 74 | (100.0) | 90 | (75.0) |
| 95% Wilson Score CI for Proportion with | (0.0, | 5.8) | (11.7, | 25.3) | (0.0, | 4.9) | (18.1, | 33.4) |
| GUSTO Severe Bleeding | | | | | | | | |
| Difference of Proportions (Bentracimab − | −17.5 | | | | −25.0 | | | |
| Control) | | | | | | | | |
| 95% Newcombe CI for Difference of | (−25.3, | −9.3) | | | (−33.4, | −16.5) | | |
| Proportions | | | | | | | | |
| Race | White | | | | Asian | | | |
| | N = 62 | | N = 237 | | N = 16 | | N = 1 | |
| Had GUSTO Severe Bleeding [a], n (%) | | | | | | | | |
| Yes | 0 | (0.0) | 50 | (21.1) | 0 | (0.0) | 0 | (0.0) |
| No | 114 | (100.0) | 187 | (78.9) | 16 | (100.0) | 1 | (100.0) |
| 95% Wilson Score CI for Proportion with | (0.0, | 3.3) | (16.4, | 26.7) | (0.0, | 19.4) | (0.0, | 79.3) |
| GUSTO Severe Bleeding | | | | | | | | |
| Difference of Proportions (Bentracimab − | −21.1 | | | | 0.0 | | | |
| Control) | | | | | | | | |
| 95% Newcombe CI for Difference of | (−26.7, | −15.4) | | | (−79.3, | 19.4) | | |
| Proportions | | | | | | | | |
| Region | North America | | | | Europe, Middle East, Africa | | | |
| | N = 117 | | N = 28 | | N = 62 | | N = 120 | |
| Had GUSTO Severe Bleeding [a], n (%) | | | | | | | | |
| Yes | 0 | (0.0) | 7 | (25.0) | 0 | (0.0) | 44 | (20.8) |
| No | 117 | (100.0) | 21 | (75.0) | 19 | (100.0) | 168 | (79.2) |
| 95% Wilson Score CI for Proportion with | (0.0, | 3.2) | (12.7, | 43.4) | (0.0, | 16.8) | (15.8, | 26.7) |
| GUSTO Severe Bleeding | | | | | | | | |
| Difference of Proportions (Bentracimab − | −25.0 | | | | −20.8 | | | |
| Control) | | | | | | | | |
| 95% Newcombe CI for Difference of | (−43.4, | −12.3) | | | (−26.7, | −3.2) | | |
| Proportions | | | | | | | | |

Abbreviations: CI = Confidence Interval.
[a] Experienced GUSTO severe bleeding within 24-hours from the start of surgery.

### All-Cause Mortality Efficacy Results

### Secondary Efficacy Analysis: All-Cause Mortality (Major Bleeding Analysis Set)

Results of the analysis of the proportion of patients in the Major Bleeding Analysis Set who died due to any cause within 38 days of major bleeding are in Table 25. Observed all-cause mortality was 9.8% (4/41) in bentracimab patients and 10.4% (14/134) in PLATO Control prior to balancing the groups across baseline characteristics. Using weighted logistic regression with sIPTW propensity score weights to balance patient characteristics between the treatment groups and adjusting for type of bleed (procedure-related, spontaneous ICH, and spontaneous non-ICH) and days off ticagrelor (0, 1 or 2), the estimates for all-cause mortality were 3.6% for bentracimab patients and 11.3% for PLATO control. This indicates risk difference (bentracimab −PLATO control) in all-cause mortality of 7.7%, 95% CI: −16.4, 1.0), P=0.0830. This result suggests less all-cause mortality in bentracimab patients.

TABLE 25
Proportion of Patients Who Died Due to Any Cause using sIPTW
Propensity Score Weighting (Major Bleeding Analysis Set)

| | Phase 3 | | PLATO | |
|---|---|---|---|---|
| | Bentracimab | | Control | |
| | (N = 41) | | (N = 134) | |
| Died within 38 Days of Major | | | | |
| Bleeding, n (%) | | | | |
| Yes | 4 | (9.8) | 14 | (10.4) |
| No | 37 | (90.2) | 120 | (89.6) |
| Estimated Risk of Death[a] | 3.6 | | 11.3 | |
| 95% CI | (0.8, | 14.9) | (5.5, | 21.8) |
| Risk Difference (Bentracimab − | −7.7 | | | |
| Control) | | | | |
| 95% CI | (−16.4, | 1.0) | | |

| | |
|---|---|
| P-value | 0.0830 |

Abbreviations: CI = Confidence Interval; sIPTW = Stabilized Inverse Probability of Treatment Weighting.
[a]Risk and risk difference are estimated by weighted logistic regression with sIPTW propensity score weights. The model predicts risk of death within 38 days of major bleeding with treatment group, type of bleed (fatal or life-threatening: procedure-related, spontaneous ICH, or spontaneous non-ICH), and days off ticagrelor prior to onset of major bleeding (0, 1, or 2) as explanatory variables.

### Sensitivity Analyses of Secondary Efficacy Endpoint (Major Bleeding Analysis Set)

A sensitivity analysis using AIPW propensity score weighting showed that estimated risks of all-cause mortality were 8.7% and 12.3% for the bentracimab and PLATO control patients, respectively (Table 26). The risk difference (bentracimab−control) was −3.6, 95% CI: −9.8, 2.6). While not statistically significant, this analysis is consistent with a trend for lower mortality in bentracimab patients.

TABLE 26
Sensitivity Analysis of Proportion of Patients
Who Died Due to Any Cause using AIPW Propensity
Score Weighting (Major Bleeding Analysis Set)

| | Phase 3 | | PLATO | |
|---|---|---|---|---|
| | Bentracimab | | Control | |
| | (N = 41) | | (N = 134) | |
| Died within 38 Days of Major | | | | |
| Bleeding, n (%) | | | | |
| Yes | 4 | (9.8) | 14 | (10.4) |
| No | 37 | (90.2) | 120 | (89.6) |
| Estimated Risk of Death [a] | 8.7 | | 12.3 | |
| 95% CI | (4.7, | 12.7) | (7.2, | 17.3) |
| Risk Difference (Bentracimab − | −3.6 | | | |
| Control) | | | | |
| 95% CI | (−9.8, | 2.6) | | |

| | |
|---|---|
| p-value | 0.2553 |

Abbreviations: AIPW = Augmented Inverse Probability Weighting; CI = Confidence Interval.
[a] Risk and risk difference are estimated by using augmented inverse probability weights (AIPW). In addition to a propensity score model, the AIPW estimation method incorporates a model for the outcome variable death. The AIPW estimation method is doubly robust and provides unbiased estimates for the Average Treatment Effect (ATE) even if one of the outcome or propensity score models is misspecified. The propensity score model includes effects for type of bleed (fatal or life-threatening: procedure-related, spontaneous ICH, or spontaneous non-ICH), days off ticagrelor prior to bleed (0, 1, or 2), median aspirin dose 7 days prior to surgery (0 mg or > 0 mg), sex (male or female), age (<=65 or >65), and region (North America or Other - includes Europe, Middle East, and Africa). The outcome model includes an effect for type of bleed (fatal or life-threatening: procedure-related, spontaneous ICH, or spontaneous non-ICH) and days off ticagrelor prior to bleed (0, 1, or 2).

Table 27 presents results for the sensitivity analysis of all-cause mortality in major bleeders when ATT propensity score weighting is applied. Here the estimated risks of all-cause mortality were 2.0% and 2.5% for the bentracimab and control patients, respectively (risk difference of −0.5%).

TABLE 27
Sensitivity Analysis of Proportion of Patients
Who Died Due to Any Cause using ATT Propensity
Score Weighting (Major Bleeding Analysis Set)

| | Phase 3 | | PLATO | |
|---|---|---|---|---|
| | Bentracimab | | Control | |
| | (N = 41) | | (N = 134) | |
| Died within 38 Days of Major | | | | |
| Bleeding, n (%) | | | | |
| Yes | 4 | (9.8) | 14 | (10.4) |
| No | 37 | (90.2) | 120 | (89.6) |
| Estimated Risk of Death[a] | 2.0 | | 2.5 | |
| 95% CI | (0.1, | 35.1) | (0.1, | 42.5) |
| Risk Difference (Bentracimab − | −0.5 | | | |
| Control) | | | | |
| 95% CI | (−5.8, | 4.7) | | |

| | |
|---|---|
| P-value | 0.8360 |

Abbreviations: ATT = Average Treatment Effect of Treated; CI = Confidence Interval.
[a]Risk and risk difference are estimated by weighted logistic regression with ATT propensity score weights. The model predicts risk of death within 38 days of major bleeding with treatment group, type of bleed (fatal or life-threatening: procedure-related, spontaneous ICH, or spontaneous non-ICH), and region (North America or Other - includes Europe, Middle East, and Africa) as explanatory variables.

The analysis using sIPTW propensity score weighting and the sensitivity analyses using AIPW and ATT weighting, all showed a lower risk of all-cause mortality when patients were treated with bentracimab. With sIPTW weighting, the estimated effect was a 7.7% reduction in the risk of death. Although the results for this endpoint did not reach statistical significance, this may be due in part to several reasons. Only 41 bentracimab-treated patients were eligible for these analyses, reducing the statistical power. Review of the demographic and baseline characteristics of major bleeding patients shows further treatment group imbalances in characteristics that were not pre-planned for inclusion in the propensity score models. These include prior myocardial infarction (63.4% bentracimab vs. 23.9% control), prior CABG (75.6% bentracimab vs. 18.7% control), and hemoglobin ≤11 g/dL (73.7% bentracimab vs. 10.6% control). Each of these suggest the bentracimab-treated patients in the Phase 3 study had more serious underlying medical conditions than the PLATO control patients. For this reason, post hoc analyses are planned where these factors will be added to the propensity score models and balanced between the treatment groups via propensity score weighting. This may result in a greater estimated risk reduction for death in patients treated with bentracimab. Finally, there may be contributing causes of mortality in these acute ill subjects in addition to ticagrelor-enhanced bleeding.

### Subset Analyses of Secondary Efficacy Endpoint (Major Bleeding Analysis Set) Type of Major Bleed

All-cause mortality rates were lower in bentracimab patients compared to PLATO control patients within each of the types of major bleeds: procedure-related bleeding, spontaneous ICH, spontaneous non-ICH, and spontaneous major bleeding (ICH and non-ICH combined). Differences in all-cause mortality rates were most pronounced in patients with spontaneous ICH (Table 28).

TABLE 28
Proportion of Patients Who Died Due to Any Cause by Fatal or Life-
Threatening Major Bleeding Type (Major Bleeding Analysis Set)

| Major Bleeding Subgroup | Phase 3 Bentracimab | | PLATO Control | |
|---|---|---|---|---|
| Procedure-related | N = 16 | | N = 82 | |
| Died within 38 Days of Major Bleeding, n (%) | | | | |
| Yes | 0 | (0.0) | 2 | (2.4) |
| No | 16 | (100.0) | 80 | (97.6) |
| 95% Wilson Score CI for Proportion who Died | (0.0, | 19.4) | (0.7, | 8.5) |
| Difference of Proportions (Bentracimab − Control) | −2.4 | | | |
| 95% Newcombe CI for Difference of Proportions | (−8.5, 17.0) | | | |
| Spontaneous ICH | N = 15 | | N = 14 | |
| Died within 38 Days of Major Bleeding, n (%) | | | | |
| Yes | 4 | (26.7) | 7 | (50.0) |
| No | 11 | (73.3) | 7 | (50.0) |
| 95% Wilson Score CI for Proportion who Died | (10.9, | 52.0) | (26.8, | 73.2) |
| Difference of Proportions (Bentracimab − Control) | −23.3 | | | |
| 95% Newcombe CI for Difference of Proportions | (−51.4, 11.0) | | | |
| Spontaneous Non-ICH | N = 10 | | N = 38 | |
| Died within 38 Days of Major Bleeding, n (%) | | | | |
| Yes | 0 | (0.0) | 5 | (13.2) |
| No | 10 | (100.0) | 33 | (86.8) |
| 95% Wilson Score CI for Proportion who Died | (0.0, | 27.8) | (5.8, | 27.3) |
| Difference of Proportions (Bentracimab − Control) | −13.2 | | | |
| 95% Newcombe CI for Difference of Proportions | (−27.3, 15.6) | | | |
| Spontaneous ICH and non-ICH Combined | N = 25 | | N = 52 | |
| Died within 38 Days of Major Bleeding, n (%) | | | | |
| Yes | 4 | (16.0) | 12 | (23.1) |
| No | 21 | (84.0) | 40 | (76.9) |
| 95% Wilson Score CI for Proportion who Died | (6.4, | 34.7) | (13.7, | 36.1) |

| | |
|---|---|
| Difference of Proportions (Bentracimab − Control) | −7.1 |
| 95% Newcombe CI for Difference of Proportions | (−23.3, 13.8) |

Abbreviations: CI = confidence interval; ICH = intracranial hemorrhage.
Sources: Table 14.2.3.2.5, Comparison of bentracimab to external control

### Other Subset Analyses of the Secondary Efficacy Endpoint

Table 29 summarizes all-cause mortality in the major bleeding analysis set according to age, sex, race, and geographical region. Absolute differences in all-cause mortality rates between bentracimab patients and PLATO control patients were ≤9.4% in all comparisons, with the largest difference favoring bentracimab among females. All 95% CIs around point estimates for mortality rate differences between bentracimab and PLATO control patients included zero.

TABLE 29
Proportion of Patients Who Died Due to Any Cause by Age, Sex, Race, and Region (Major Bleeding Analysis Set)

| | Phase 3 Bentracimab | | PLATO Control | | Phase 3 Bentracimab | | PLATO Control | |
|---|---|---|---|---|---|---|---|---|
| Age | ≤65 years | | | | >65 years | | | |
| | N = 20 | | N = 56 | | N = 21 | | N = 78 | |
| Died within 38 Days of Major Bleeding [a], n (%) | | | | | | | | |
| Yes | 1 | (5.0) | 3 | (5.4) | 3 | (14.3) | 11 | (14.1) |
| No | 19 | (95.0) | 53 | (94.6) | 18 | (85.7) | 67 | (85.9) |
| 95% Wilson Score CI for Proportion who Died | (0.9, | 23.6) | (1.8, | 14.6) | (5.0, | 34.6) | (8.1, | 23.5) |
| Difference of Proportions (Bentracimab − Control) | −0.4 | | | | 0.2 | | | |
| 95% Newcombe CI for Difference of Proportions | (−10.5, | 18.6) | | | (−13.1, | 21.4) | | |
| Sex | Female | | | | Male | | | |
| | N = 11 | | N = 32 | | N = 30 | | N = 102 | |
| Died within 38 Days of Major Bleeding [a], n (%) | | | | | | | | |
| Yes | 0 | (0.0) | 3 | (9.4) | 4 | (13.3) | 11 | (10.8) |
| No | 11 | (100.0) | 29 | (90.6) | 26 | (86.7) | 91 | (89.2) |
| 95% Wilson Score CI for Proportion who Died | (0.0, | 25.9) | (3.2, | 24.2) | (5.3, | 29.7) | (6.1, | 18.3) |
| Difference of Proportions (Bentracimab − Control) | −9.4 | | | | 2.5 | | | |
| 95% Newcombe CI for Difference of Proportions | (−24.2, | 17.2) | | | (−8.4, | 19.5) | | |
| Race | White | | | | Black | | | |
| | N = 37 | | N = 133 | | N = 2 | | N = 1 | |
| Died within 38 Days of Major Bleeding [a], n (%) | | | | | | | | |
| Yes | 4 | (10.8) | 14 | (10.5) | 0 | (0.0) | 0 | (0.0) |
| No | 33 | (89.2) | 119 | (89.5) | 2 | (100.0) | 1 | (100.0) |
| 95% Wilson Score CI for Proportion who Died | (4.3, | 24.7) | (6.4, | 16.9) | (0.0, | 65.8) | (0.0, | 79.3) |
| Difference of Proportions (Bentracimab − Control) | 0.3 | | | | 0.0 | | | |
| 95% Newcombe CI for Difference of Proportions | (−8.8, | 14.8) | | | (−79.3, | 65.8) | | |
| Region | North America | | | | Europe, Middle East, Africa | | | |
| | N = 30 | | N = 11 | | N = 11 | | N = 123 | |
| Died within 38 Days of Major Bleeding [a], n (%) | | | | | | | | |
| Yes | 2 | (6.7) | 0 | (0.0) | 2 | (18.2) | 14 | (11.4) |
| No | 28 | (93.3) | 11 | (100.0) | 9 | (81.8) | 109 | (88.6) |
| 95% Wilson Score CI for Proportion who Died | (1.8, | 21.3) | (0.0, | 25.9) | (5.1, | 47.7) | (6.9, | 18.2) |
| Difference of Proportions (Bentracimab − Control) | 6.7 | | | | 6.8 | | | |
| 95% Newcombe CI for Difference of Proportions | (−19.7, | 21.3) | | | (−7.9, | 36.7) | | |

Abbreviation: CI = confidence interval.

### All-Cause Mortality (Urgent Surgery Analysis Set)

Results of the analysis of the proportion of patients in the urgent surgery analysis set who died due to any cause within 38 days of urgent surgery is presented in Table 30. All-cause mortality rates were lower in bentracimab patients compared to PLATO control patients; (2.9% vs. 9.6%; difference: −6.6%; 95% CI: −11.4%, −1.3%).

TABLE 30
Proportion of Patients Who Died Due to
Any Cause (Urgent Surgery Analysis Set)

| | |
|---|---|
| Phase 3 | PLATO |
| Bentracimab | Control |
| (N = 136) | (N = 240) |

| | | | | |
|---|---|---|---|---|
| Died within 38 Days of | | | | |
| Urgent Surgery [a], n (%) | | | | |
| Yes | 4 | (2.9) | 23 | (9.6) |
| No | 132 | (97.1) | 217 | (90.4) |
| 95% Wilson Score CI for | (1.1, | 7.3) | (6.5, | 14.0) |
| Proportion who Died | | | | |

| | |
|---|---|
| Difference of Proportions | −6.6 |
| (Bentracimab − Control) | |

| | | |
|---|---|---|
| 95% Newcombe CI for | (−11.4, | −1.3) |
| Difference of Proportions | | |

Abbreviation: CI = confidence interval.

Subset Analyses of all-Cause Mortality (Urgent Surgery Analysis Set)

Table 31 summarizes all-cause mortality in the urgent surgery analysis set according to age, sex, race, and geographical region. Absolute differences in all-cause mortality rates between bentracimab and PLATO control patients were as high as 14.5%, favoring bentracimab patients in all comparisons except for one involving a paucity of Asians. Point estimates for mortality rate differences between bentracimab and PLATO control patients were associated with 95% CIs that did not include zero in CABG patients, patients >65 years of age, females, and Whites.

TABLE 31
Proportion of Patients Who Died Due to Any Cause by Age, Sex, Race, and Region (Urgent Surgery Analysis Set)

| | Phase 3 Bentracimab | | PLATO Control | | Phase 3 Bentracimab | | PLATO Control | |
|---|---|---|---|---|---|---|---|---|
| Urgent Surgery Subgroup | CABG | | | | Non-CABG | | | |
| | N = 111 | | N = 102 | | N = 25 | | N = 138 | |
| Died within 38 Days of Major Bleeding [a], n (%) | | | | | | | | |
| Yes | 2 | (1.8) | 8 | (7.8) | 2 | (8.0) | 15 | (10.9) |
| No | 109 | (98.2) | 94 | (92.2) | 23 | (92.0) | 123 | (89.1) |
| 95% Wilson Score CI for Proportion who Died | (0.5, | 6.3) | (4.0, | 14.7) | (2.2, | 25.0) | (6.7, | 17.2) |
| Difference of Proportions (Bentracimab − Control) | −6.0 | | | | −2.9 | | | |
| 95% Newcombe CI for Difference of Proportions | (−13.0, | −0.1) | | | (−11.4, | 14.6) | | |
| Age | ≤65 years | | | | >65 years | | | |
| | N = 62 | | N = 120 | | N = 74 | | N = 120 | |
| Died within 38 Days of Major Bleeding [a], n (%) | | | | | | | | |
| Yes | 2 | (3.2) | 8 | (6.7) | 2 | (2.7) | 15 | (12.5) |
| No | 60 | (96.8) | 112 | (93.3) | 72 | (97.3) | 105 | (87.5) |
| 95% Wilson Score CI for Proportion who Died | (0.9, | 11.0) | (3.4, | 12.6) | (0.7, | 9.3) | (7.7, | 19.6) |
| Difference of Proportions (Bentracimab − Control) | −3.4 | | | | −9.8 | | | |
| 95% Newcombe CI for Difference of Proportions | (−9.8, | 5.0) | | | (−17.2, | −1.6) | | |
| Sex | Female | | | | Male | | | |
| | N = 28 | | N = 76 | | N = 108 | | N = 164 | |
| Died within 38 Days of Major Bleeding [a], n (%) | | | | | | | | |
| Yes | 0 | (0.0) | 11 | (14.5) | 4 | (3.7) | 12 | (7.3) |
| No | 28 | (100.0) | 65 | (85.5) | 104 | (96.3) | 152 | (92.7) |
| 95% Wilson Score CI for Proportion who Died | (0.0, | 12.1) | (8.3, | 24.1) | (1.4, | 9.1) | (4.2, | 12.4) |
| Difference of Proportions (Bentracimab − Control) | −14.5 | | | | −3.6 | | | |
| 95% Newcombe CI for Difference of Proportions | (−24.1, | −0.9) | | | (−9.1, | 2.6) | | |
| Race | White | | | | Asian | | | |
| | N = 114 | | N = 237 | | N = 16 | | N = 1 | |
| Died within 38 Days of Major Bleeding [a], n (%) | | | | | | | | |
| Yes | 2 | (1.8) | 23 | (9.7) | 1 | (6.3) | 0 | (0.0) |
| No | 112 | (98.2) | 214 | (90.3) | 15 | (93.8) | 1 | (100.0) |
| 95% Wilson Score CI for Proportion who Died | (0.5, | 6.2) | (6.6, | 14.1) | (1.1, | 28.3) | (0.0, | 79.3) |
| Difference of Proportions (Bentracimab − Control) | −8.0 | | | | 6.2 | | | |
| 95% Newcombe CI for Difference of Proportions | (−12.6, | −2.5) | | | (−73.3, | 28.3) | | |
| Region | North America | | | | Europe, Middle East, Africa | | | |
| | N = 117 | | N = 28 | | N = 19 | | N = 212 | |
| Died within 38 Days of Major Bleeding [a], n (%) | | | | | | | | |
| Yes | 4 | (3.4) | 2 | (7.1) | 0 | (0.0) | 21 | (9.9) |
| No | 113 | (96.6) | 26 ( | 92.9) | 19 | (100.0) | 191 | (90.1) |
| 95% Wilson Score CI for Proportion who Died | (1.3, | 8.5) | (2.0, | 22.6) | (0.0, | 16.8) | (6.6, | 14.7) |
| Difference of Proportions (Bentracimab − Control) | −3.7 | | | | −9.9 | | | |
| 95% Newcombe CI for Difference of Proportions | (−19.4, | 3.5) | | | (−14.7, | 7.2) | | |

Abbreviation: CI = confidence interval.

### All-Cause Mortality (Combined Analysis Set)

In the combined analysis set, all-cause mortality rates were lower in bentracimab compared to PLATO control patients (4.5% vs. 9.9%; difference: −5.4%; 95% CI: −9.5%, −0.5%), consistent with results in the urgent surgery analysis set.

Subset Analyses of all-Cause Mortality (Combined Analysis Set)

Absolute differences in all-cause mortality rates between bentracimab and PLATO control patients were as high as 13.0%, favoring bentracimab patients in all comparisons except for those involving a paucity of Asians and Blacks. Point estimates for mortality rate differences between bentracimab and PLATO control patients were associated with 95% Cis that did not include zero in patients >65 years of age, females, and Whites. These results are consistent with those obtained in the urgent surgery analysis set.

### Example 3: Extended Bentracimab Phase 3 Compared to PLATO Control Efficacy Results

This Example describes additional comparative analysis between the Bentracimab Phase 3 study and the PLATO study, as described in Example 2.

### Results

Treatment with Bentracimab Reduced Risk of First Transfusion in Major Bleeding Patients

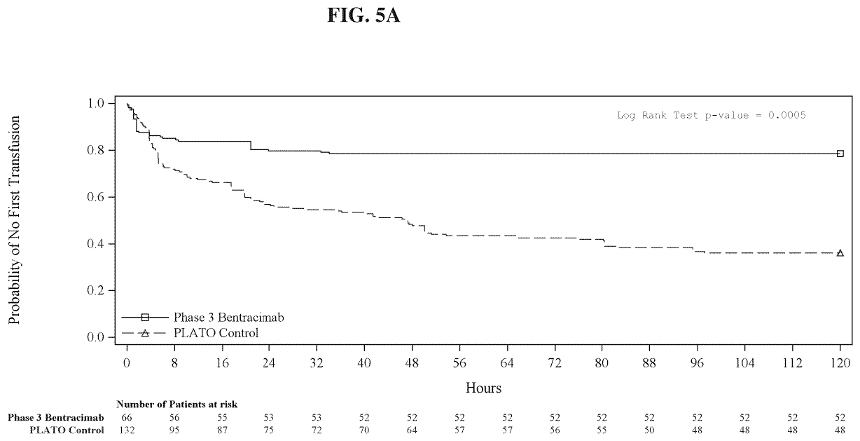

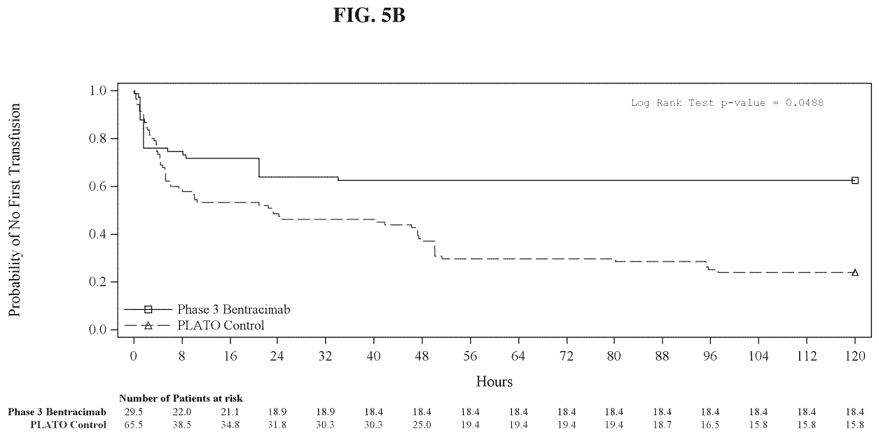

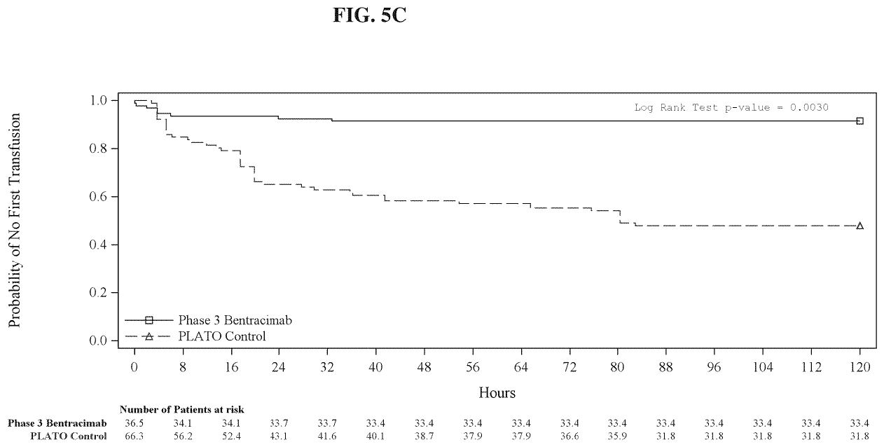

FIGS. 5A-5C show that major bleeding patients who received bentracimab in the Phase 3 trial had a reduced in risk of receiving a first blood transfusion within 120 hours after receiving bentracimab compared to control patients in the PLATO study who did not receive bentracimab. The reduction in probability of receiving a first transfusion was statistically significant (p-value=0.0005) between patients with major bleeding who received bentracimab compared to those in the PLATO control (FIG. 5A). Significant reduction in risk receiving a first blood transfusion within 120 hours after receiving bentracimab was also observed in subgroup analysis of patients with procedure-related major bleeding (p-value=0.0488; FIG. 5B) and patients with spontaneous major bleeding (p-value=0.0030; FIG. 5C).

In this analysis, the start for PLATO patients was set to the median time from start of major bleeding to bentracimab infusion in each Phase 3 major bleeding subgroup: 0.73 hours for procedure-related major bleeds, 6.55 hours for spontaneous intracranial hemorrhage (ICH) bleeds, and 20.32 hours for spontaneous non-ICH bleeds. Deaths occurring in this interval and prior to first transfusion were counted as events. Patients were censored if they did not have a first transfusion or die or discontinue the study without having a first transfusion with red blood cells or whole blood within this interval. Kaplan-Meier estimates were obtained using SAS Proc Lifetest with ‘weight’ statement using sIPTW propensity score weights to create a weighted sample in which the distribution of measured baseline covariates was independent of treatment assignment. Adjusted survival curves based on the weighted sample are presented. Number of patients at risk at each timepoint as based on the weighted sample.

The risk analyses of bentracimab-treated Phase 3 Major Bleeding patients compared to PLATO controls for requiring a first transfusion are presented in Tables 32-36. For major bleeding patients, the risk of bentracimab-treated patients for receiving a first transfusion was significantly lower compared to control (risk ratio=0.259, 95% CI=0.105, 0.413, p-value <0.0001; Table 32). In subgroup analysis for procedure-related major bleeding patients, the risk of bentracimab-treated patients for receiving a first transfusion was also significantly lower compared to control (risk ratio=0.543, 95% CI=0.286, 0.800, p-value 0.0005; Table 33). Similar benefit for reducing risk of bentracimab-treated patients for receiving a first transfusion was observed in spontaneous major bleeding patients (risk ratio=0.072, 95% CI=−0.045, 0.190, p-value <0.0001; Table 34). Further subgroup analysis was performed for patients with spontaneous ICH major bleeding and spontaneous non-ICH (e.g., gastrointestinal) major bleeding. Tables 35-36 show that in both of these subgroups the risk of bentracimab-treated patients for receiving a first transfusion was also lower compared to control. This difference was statistically significant in the spontaneous non-ICH group (risk difference=−63.0, 95% CI=−81.2, −44.8, p-value <0.0001; Table 35). The reduced risk in bentracimab patients did not reach statistical significance in the spontaneous ICH group (risk difference=−26.4, 95% CI=−56.0, 3.3, p-value 0.0818; Table 35), likely due to small sample size.

Similar to the analysis shown in FIGS. 5A-5C, the start for PLATO patients was set to median time from start of major bleeding to bentracimab infusion in each Phase 3 major bleeding subgroup: 0.73 hours for procedure-related major bleeds, 6.55 hours for spontaneous ICH bleeds, and 20.32 hours for spontaneous non-ICH bleeds. Patients who did not have a transfusion with red blood cells or whole blood, die, or discontinue after start of major bleeding and prior to bentracimab infusion in Phase 3 or the equivalent start in PLATO were included in the risk set. To account for missing data, “yes” under “required first transfusion from start of bentracimab infusion or equivalent start in PLATO” included patients who died prior to first transfusion in this interval. Risks, risk difference, and risk ratio were estimated by weighted logistic regression with sIPTW propensity score weights. The model predicted risk of first transfusion or death with treatment group and type of bleed (spontaneous ICH or spontaneous non-ICH) as explanatory variables.

TABLE 32
Proportion of patients who required first transfusion with
red blood cells or whole blood after start of bentracimab
infusion in Phase 3 or equivalent start in PLATO to <=120
hours after start of major bleeding using sIPTW propensity
score weighting (Major Bleeding patients)

| | Phase 3 | | PLATO | |
|---|---|---|---|---|
| | Bentracimab | | Control | |
| | (N = 68) | | (N = 134) | |
| Required First Transfusion from | | | | |
| Start of Bentracimab | | | | |
| Infusion or Equivalent Start in | | | | |
| PLATO to <=120 Hours, n (%) | | | | |
| At Risk for First Transfusion in | 58 | | 114 | |
| this Interval | | | | |
| Yes | 12 | (20.7) | 77 | (67.5) |
| No | 46 | (79.3) | 37 | (32.5) |
| Estimated Risk of First Transfusion | 15.8 | | 60.8 | |
| 95% CI | (8.5, | 27.3) | (49.9, | 70.7) |
| Risk Difference (Bentracimab − | −45.0 | | | |
| Control) | | | | |
| 95% CI | (−58.3, | −31.8) | | |
| p-value | <0.0001 | | | |
| Risk Ratio (Bentracimab/Control) | 0.259 | | | |
| 95% CI | (0.105, | 0.413) | | |

| | |
|---|---|
| p-value | <0.0001 |

TABLE 33
Proportion of patients who required first transfusion with red
blood cells or whole blood after start of bentracimab infusion
in Phase 3 or equivalent start in PLATO to <=120 hours
after start of major bleeding using sIPTW propensity score
weighting (Patients with Procedure-Related Major Bleeding)

| | Phase 3 | | PLATO | |
|---|---|---|---|---|
| | Bentracimab | | Control | |
| | (N = 21) | | (N = 82) | |
| Required First Transfusion from | | | | |
| Start of Bentracimab | | | | |
| Infusion or Equivalent Start in | | | | |
| PLATO to <=120 Hours, n (%) | | | | |
| At Risk for First Transfusion in | 19 | | 74 | |
| this Interval | | | | |
| Yes | 9 | (47.4) | 55 | (74.3) |
| No | 10 | (52.6) | 19 | (25.7) |
| Estimated Risk of First Transfusion | 41.3 | | 76.1 | |
| 95% CI | (24.6, | 60.3) | (63.7, | 85.2) |
| Risk Difference (Bentracimab − | −34.8 | | | |
| Control) | | | | |
| 95% CI | (−56.4, | −13.2) | | |
| p-value | 0.0016 | | | |
| Risk Ratio (Bentracimab/Control) | 0.543 | | | |
| 95% CI | (0.286, | 0.800) | | |

| | |
|---|---|
| p-value | 0.0005 |

TABLE 34
Proportion of patients who required first transfusion with
red blood cells or whole blood after start of bentracimab
infusion in Phase 3 or equivalent start in PLATO to <=120
hours after start of major bleeding using sIPTW propensity
score weighting (Patients with Spontaneous Major Bleeding)

| | Phase 3 | | PLATO | |
|---|---|---|---|---|
| | Bentracimab | | Control | |
| | (N = 47) | | (N = 52) | |
| Required First Transfusion from | | | | |
| Start of Bentracimab | | | | |
| Infusion or Equivalent Start in | | | | |
| PLATO to <=120 Hours, n (%) | | | | |
| At Risk for First Transfusion in | 39 | | 40 | |
| this Interval | | | | |
| Yes | 3 | (7.7) | 22 | (55.0) |
| No | 36 | (92.3) | 18 | (45.0) |
| Estimated Risk of First Transfusion | 3.9 | | 53.8 | |
| 95% CI | (0.8, | 17.8) | (39.3, | 67.8) |
| Risk Difference (Bentracimab − | −49.9 | | | |
| Control) | | | | |
| 95% CI | (−65.7, | −34.2) | | |
| p-value | <0.0001 | | | |
| Risk Ratio (Bentracimab/Control) | 0.072 | | | |
| 95% CI | (−0.045, | 0.190) | | |

| | |
|---|---|
| p-value | <0.0001 |

TABLE 35
Proportion of patients who required first transfusion with red
blood cells or whole blood after start of bentracimab infusion
in Phase 3 or equivalent start in PLATO to <=120 hours
after start of major bleeding using sIPTW propensity score
weighting (Patients with Spontaneous non-ICH Major Bleeding)

| | Phase 3 | | PLATO | |
|---|---|---|---|---|
| | Bentracimab | | Control | |
| | (N = 27) | | (N = 38) | |
| Required First Transfusion from | | | | |
| Start of Bentracimab | | | | |
| Infusion or Equivalent Start in | | | | |
| PLATO to <=120 Hours, n (%) | | | | |
| At Risk for First Transfusion in | 19 | | 27 | |
| this Interval | | | | |
| Yes | 0 | (0.0) | 17 | (63.0) |
| No | 19 | (100.0) | 10 | (37.0) |
| Estimated Risk of First Transfusion | 2.2 | | 65.2 | |
| 95% CI | (0.1, | 28.3) | (46.8, | 79.9) |
| Risk Difference (Bentracimab − | −63.0 | | | |
| Control) | | | | |
| 95% CI | (−81.2, | −44.8) | | |

| | |
|---|---|
| p-value | <0.0001 |

TABLE 36
Proportion of patients who required first transfusion with red blood cells
or whole blood after start of bentracimab infusion in Phase 3 or equivalent
start in PLATO to <=120 hours after start of major bleeding using sIPTW
propensity score weighting (Patients with Spontaneous ICH Major Bleeding)

| | Phase 3 | | PLATO | |
|---|---|---|---|---|
| | Bentracimab | | Control | |
| | (N = 20) | | (N = 14) | |
| Required First Transfusion from Start of | | | | |
| Bentracimab | | | | |
| Infusion or Equivalent Start in PLATO | | | | |
| to <= 120 Hours, n (%) | | | | |
| At Risk for First Transfusion in this Interval | 20 | | 13 | |
| Yes | 3 | (15.0) | 5 | (38.5) |
| No | 17 | (85.0) | 8 | (61.5) |
| Estimated Risk of First Transfusion | 12.9 | (2.6, 45.5) | 39.3 | (20.2, 62.4) |
| 95% CI | | | | |
| Risk Difference (Bentracimab − Control) | −26.4 | (−56.0, 3.3) | | |
| 95% CI | | | | |

| | |
|---|---|
| p-value | 0.0818 |

Further, FIGS. 5A-5C show that treatment with bentracimab extended time to first transfusion compared to PLATO controls. As shown in Table 37, the time to first transfusion or death before first transfusion was 10 hours for the 25^{th} percentile of Phase 3 Major Bleeding patients who received bentracimab compared to 3.4 hours in PLATO controls. The median of Phase 3 Major Bleeding patients was 25.5 hours (95% CI=10.8, 48.0) in PLATO controls and was not estimable in Phase 3 Major Bleeding patients who received bentracimab.

TABLE 37
Time to First Transfusion with Red Blood Cells or Whole Blood after Start of Major
Bleeding with sIPTW Propensity Score weighting (Major Bleeding Patients)

| | Phase 3 | | PLATO | |
|---|---|---|---|---|
| | Bentracimab | | Control | |
| | (N = 68) | | (N = 134) | |
| Transfusion-Free Survival, n (%) | | | | |
| Event | 22 | (32.4) | 97 | (72.4) |
| First Transfusion | 22 | (32.4) | 90 | (67.2) |
| Death before First Transfusion | 0 | | 7 | (5.2) |
| Censored | 46 | (67.6) | 37 | (27.6) |
| Time to First Transfusion or Death | | | | |
| before First Transfusion (hours) | | | | |
| 25th percentile (95% CI) | 10.0 | (NE, NE) | 3.4 | (2.0, 6.0) |
| Median (95% CI) | NE | (NE, NE) | 25.5 | (10.8, 48.0) |
| 75th percentile (95% CI) | NE | (NE, NE) | NE | (50.8, NE) |
| Min, Max | 0.0, 120.0* | | 0.0, 120.0* | |
| Probability of Being Event-free | | | | |
| 24 hours (95% CI) | 0.744 | (0.558, 0.860) | 0.514 | (0.405, 0.613) |
| | [n = 49] | | [n = 77] | |
| 48 hours (95% CI) | 0.732 | (0.545, 0.851) | 0.383 | (0.283, 0.483) |
| | [n = 48] | | [n = 53] | |
| 72 hours (95% CI) | 0.720 | (0.532, 0.842) | 0.308 | (0.218, 0.404) |
| | [n = 47] | | [n = 42] | |
| 96 hours (95% CI) | 0.720 | (0.532, 0.842) | 0.286 | (0.199, 0.379) |
| | [n = 47] | | [n = 38] | |
| 120 hours (95% CI) | 0.720 | (0.532, 0.842) | 0.269 | (0.185, 0.361) |

| | |
|---|---|
| [n = 47] | [n = 35] |

Abbreviations: CI = Confidence Interval; sIPTW = Stabilized Inverse Probability of Treatment Weighting; NE = Not Estimable.

In the Phase 3 study, the median time to bentracimab infusion after start of major bleeding was 4 hours. In Table 37, * denotes censored observation. Patients were censored if they did not have a first transfusion or die or discontinue the study without having a first transfusion with red blood cells or whole blood within 120 hours of start of major bleeding. Kaplan-Meier estimates were obtained using SAS Proc Lifetest with ‘weight’ statement using sIPTW propensity score weights to create a weighted sample in which the distribution of measured baseline covariates was independent of treatment assignment. Adjusted Kaplan-Meier estimates for the survival curves in the weighted sample are presented. n=number of patients at risk in the weighted sample at the specified timepoint.

Treatment with Bentracimab Reduced Number of Transfusions Required in Patients with Major Bleeding and Patients Requiring Urgent Surgery

In the combined urgent surgery and major bleeding patient populations, patients who received bentracimab received a statistically significantly lower number of transfusions within 120 hours of receiving bentracimab compared to PLATO control (p-value <0.0001; Table 38). Similar reduction of number of transfusions was observed in the individual major bleeding or urgent surgery patient populations as compared to PLATO control, as shown in Table 39 and Table 40, respectively. Within the major bleeding patient population, further subgroup analysis was performed for patients with procedure-related major bleeding (Table 41) and spontaneous major bleeding (Table 42), and it was observed that treatment with bentracimab significantly reduced number of transfusions in both groups as compared to PLATO control.

For analysis of the PLATO control group, equivalent start for Urgent Surgery patients was set to start of surgery and for Major Bleeding patients was set to median time to bentracimab infusion in each major bleeding subgroup: 0.73 hours for procedure-related major bleeds, 6.55 hours for spontaneous ICH bleeds, and 20.32 hours for spontaneous non-ICH bleeds. To account for missing data, the number of transfusions included number of patients who died between start of interval (bentracimab infusion or equivalent) and 120 hours after start of surgery or major bleeding and prior to first transfusion. P-value was based on Wilcoxon Two-Sample Test comparing the transfusion distributions between the groups. This analysis was not adjusted for potential confounding variables using propensity score methods.

TABLE 38
Number of transfusion with red blood cells or whole blood from
start of bentracimab infusion in Phase 3 or equivalent start
in PLATO to <=120 Hours after start of surgery in urgent
surgery patients or start of major bleeding in major bleeding
patients (combined urgent surgery and major bleeding patients)

| | Phase 3 | | PLATO | |
|---|---|---|---|---|
| | Bentracimab | | Control | |
| | (N = 209) | | (N = 374) | |
| Number of Transfusions, n (%) | | | | |
| 0 | 186 | (89.0) | 177 | (47.3) |
| 1 | 6 | (2.9) | 35 | (9.4) |
| 2 | 6 | (2.9) | 63 | (16.8) |
| 3 | 4 | (1.9) | 23 | (6.1) |
| 4 | 4 | (1.9) | 32 | (8.6) |
| 5 | 0 | (0.0) | 11 | (2.9) |
| 6 | 0 | (0.0) | 11 | (2.9) |
| 7 | 1 | (0.5) | 6 | (1.6) |
| 8 | 0 | (0.0) | 5 | (1.3) |
| 9 | 1 | (0.5) | 1 | (0.3) |
| 11 | 0 | (0.0) | 4 | (1.1) |
| 12 | 0 | (0.0) | 1 | (0.3) |
| 13 | 0 | (0.0) | 3 | (0.8) |
| 15 | 0 | (0.0) | 1 | (0.3) |
| 17 | 0 | (0.0) | 1 | (0.3) |
| 22 | 1 | (0.5) | 0 | (0.0) |
| n | 209 | | 374 | |
| Mean (SD) | 0.4 | (1.85) | 1.9 | (2.70) |

| | | |
|---|---|---|
| Median | 0.0 | 1.0 |
| Min, Max | 0, 22 | 0, 17 |
| Wilcoxon Test p-value | <0.0001 | |

TABLE 39
Number of transfusion with red blood cells or whole
blood from start of bentracimab infusion in Phase
3 or equivalent start in PLATO to <=120 Hours
after start of major bleeding (major bleeding patients)

| | Phase 3 | | PLATO | |
|---|---|---|---|---|
| | Bentracimab | | Control | |
| | (N = 68) | | (N = 134) | |
| Number of Transfusions, n (%) | | | | |
| 0 | 52 | (76.5) | 44 | (32.8) |
| 1 | 4 | (5.9) | 16 | (11.9) |
| 2 | 4 | (5.9) | 25 | (18.7) |
| 3 | 4 | (5.9) | 13 | (9.7) |
| 4 | 2 | (2.9) | 19 | (14.2) |
| 5 | 0 | (0.0) | 5 | (3.7) |
| 6 | 0 | (0.0) | 3 | (2.2) |
| 7 | 1 | (1.5) | 3 | (2.2) |
| 8 | 0 | (0.0) | 3 | (2.2) |
| 9 | 0 | (0.0) | 1 | (0.7) |
| 11 | 0 | (0.0) | 1 | (0.7) |
| 15 | 0 | (0.0) | 1 | (0.7) |
| 22 | 1 | (1.5) | 0 | (0.0) |
| n | 68 | | 134 | |
| Mean (SD) | 0.9 | (2.91) | 2.3 | (2.52) |

| | | |
|---|---|---|
| Median | 0.0 | 2.0 |
| Min, Max | 0, 22 | 0, 15 |
| Wilcoxon Test p-value | <0.0001 | |

TABLE 40
Number of transfusion with red blood cells or whole blood from start
of bentracimab infusion in Phase 3 or equivalent start in PLATO
to <=120 Hours after start of surgery (urgent surgery patients)

| | Phase 3 | | PLATO | |
|---|---|---|---|---|
| | Bentracimab | | Control | |
| | (N = 141) | | (N = 240) | |
| Number of Transfusions, n (%) | | | | |
| 0 | 134 | (95.0) | 133 | (55.4) |
| 1 | 2 | (1.4) | 19 | (7.9) |
| 2 | 2 | (1.4) | 38 | (15.8) |
| 3 | 0 | (0.0) | 10 | (4.2) |
| 4 | 2 | (1.4) | 13 | (5.4) |
| 5 | 0 | (0.0) | 6 | (2.5) |
| 6 | 0 | (0.0) | 8 | (3.3) |
| 7 | 0 | (0.0) | 3 | (1.3) |
| 8 | 0 | (0.0) | 2 | (0.8) |
| 9 | 1 | (0.7) | 0 | (0.0) |
| 11 | 0 | (0.0) | 3 | (1.3) |
| 12 | 0 | (0.0) | 1 | (0.4) |
| 13 | 0 | (0.0) | 3 | (1.3) |
| 17 | 0 | (0.0) | 1 | (0.4) |
| n | 141 | | 240 | |
| Mean (SD) | 0.2 | (0.92) | 1.6 | (2.78) |

| | | |
|---|---|---|
| Median | 0.0 | 0.0 |
| Min, Max | 0, 9 | 0, 17 |
| Wilcoxon Test p-value | <0.0001 | |

TABLE 41
Number of transfusion with red blood cells or whole blood
from start of bentracimab infusion in Phase 3 or equivalent
start in PLATO to <=120 Hours after start of major
bleeding (patients with procedure-related major bleeding)

| | Phase 3 | | PLATO | |
|---|---|---|---|---|
| | Bentracimab | | Control | |
| | (N = 21) | | (N = 82) | |
| Number of Transfusions, n (%) | | | | |
| 0 | 12 | (57.1) | 21 | (25.6) |
| 1 | 2 | (9.5) | 8 | (9.8) |
| 2 | 3 | (14.3) | 16 | (19.5) |
| 3 | 2 | (9.5) | 10 | (12.2) |
| 4 | 1 | (4.8) | 14 | (17.1) |
| 5 | 0 | (0.0) | 4 | (4.9) |
| 6 | 0 | (0.0) | 3 | (3.7) |
| 7 | 1 | (4.8) | 3 | (3.7) |
| 8 | 0 | (0.0) | 1 | (1.2) |
| 9 | 0 | (0.0) | 1 | (1.2) |
| 11 | 0 | (0.0) | 1 | (1.2) |
| n | 21 | | 82 | |
| Mean (SD) | 1.2 | (1.83) | 2.6 | (2.37) |

| | | |
|---|---|---|
| Median | 0.0 | 2.0 |
| Min, Max | 0, 7 | 0, 11 |
| Exact Wilcoxon Test p-value | 0.0046 | |

TABLE 42
Number of transfusion with red blood cells or whole blood
from start of bentracimab infusion in Phase 3 or equivalent
start in PLATO to <=120 Hours after start of major
bleeding (patients with spontaneous major bleeding)

| | Phase 3 | | PLATO | |
|---|---|---|---|---|
| | Bentracimab | | Control | |
| | (N = 47) | | (N = 52) | |
| Number of Transfusions, n (%) | | | | |
| 0 | 40 | (85.1) | 23 | (44.2) |
| 1 | 2 | (4.3) | 8 | (15.4) |
| 2 | 1 | (2.1) | 9 | (17.3) |
| 3 | 2 | (4.3) | 3 | (5.8) |
| 4 | 1 | (2.1) | 5 | (9.6) |
| 5 | 0 | (0.0) | 1 | (1.9) |
| 8 | 0 | (0.0) | 2 | (3.8) |
| 15 | 0 | (0.0) | 1 | (1.9) |
| 22 | 1 | (2.1) | 0 | (0.0) |
| n | 47 | | 52 | |
| Mean (SD) | 0.8 | (3.29) | 1.8 | (2.69) |

| | | |
|---|---|---|
| Median | 0.0 | 1.0 |
| Min, Max | 0, 22 | 0, 15 |
| Exact Wilcoxon Test p-value | <0.0001 | |

Treatment with Bentracimab Reduced Risk of Requiring Surgery to Treat Bleeding after an Index Urgent Surgery or Major Bleeding Event

Among both the urgent surgery and major bleeding patient populations, patients who received bentracimab had a statistically significantly lower risk of requiring surgery to treat bleeding within 48 hours after the index urgent surgery event or onset of the index major bleeding event (Table 43). The risk difference (bentracimab −control) of −4.1 (95% CI: (−7.2, −1.0)) indicates a statistically significant reduction in risk of requiring surgery to treat bleeding in bentracimab-treated patients, p-value=0.0104.

For PLATO patients, surgeries to treat bleeding included those within 48 hours after the start of the index surgery or major bleeding event. For Phase 3 patients, surgeries to treat bleeding included those within 48 hours after the end of bentracimab infusion given for the index surgery or major bleeding event. Risk and risk difference were estimated by weighted logistic regression using Firth's penalized likelihood with sIPTW propensity score weights. The model predicted risk of surgery to treat bleeding within 48 hours with treatment group, subgroup (urgent surgery or major bleeding), treatment group by subgroup interaction, and type of surgery or bleed nested within subgroup (CABG or non-CABG for urgent surgery; procedure-related, spontaneous ICH, or spontaneous non-ICH for major bleeding) as explanatory variables.

TABLE 43
Analysis of Proportion of Patients Requiring Surgery to
Treat Bleeding within 48 hours After the Index Urgent Surgery
Event or Onset of Index Major Bleeding Event using SIPTW
Propensity Score Weighting (Combined Analysis Set)

| | Phase 3 | | PLATO | |
|---|---|---|---|---|
| | Bentracimab | | Control | |
| | (N = 209) | | (N = 374) | |
| Required Surgery within 48 Hours, | | | | |
| n (%) | | | | |
| Yes | 2 | (1.0) | 29 | (7.8) |
| No | 207 | (99.0) | 345 | (92.2) |

| | | |
|---|---|---|
| Estimated Risk of Surgery | 1.2 | 5.3 |
| 95% CI | (0.3, 5.1) | (3.0, 9.0) |
| Risk Difference (Bentracimab − | −4.1 | |
| Control) | | |
| 95% CI | (−7.2, −1.0) | |
| p-value | 0.0104 | |

CI = Confidence Interval; sIPTW = Stabilized Inverse of Treatment Weighting.

A lower risk of requiring surgery to treat bleeding within 48 hours after the index urgent surgery was observed in the Phase 3 urgent surgery group compared to PLATO control. The difference did not reach statistical significance (p-value=0.0912; Table 44), which may be due to a relatively small sample size being underpowered to show statistical difference, and low numbers of patients in even the PLATO control who required a second surgery to treat rebleeding after the index surgery (2.9%).

TABLE 44
Analysis of Proportion of Patients Requiring Surgery to Treat Bleeding
within 48 hours After the Index Urgent Surgery Event using sIPTW
Propensity Score Weighting (Urgent Surgery Patients)

| | Phase 3 | | PLATO | |
|---|---|---|---|---|
| | Bentracimab | | Control | |
| | (N = 141) | | (N = 240) | |
| Required Surgery within 48 Hours, | | | | |
| n (%) | | | | |
| Yes | 0 | (0.0) | 7 | (2.9) |
| No | 141 | (100.0) | 233 | (97.1) |

| | | |
|---|---|---|
| Estimated Risk of Surgery | 0.3 | 2.0 |
| 95% CI | (0.0, 4.4) | (0.7, 5.4) |
| Risk Difference (Bentracimab − | −1.7 | |
| Control) | | |
| 95% CI | (−3.8, 0.3) | |
| p-value | 0.0912 | |

In the Phase 3 Major Bleeding group, the risk ratio of 0.366 (95% CI: (−0.051, 0.783)) indicates a statistically significant reduction in risk of requiring surgery to treat bleeding in bentracimab-treated patients compared to PLATO control, p-value=0.0029 (Table 45).

TABLE 45
Analysis of Proportion of Patients Requiring Surgery to Treat Bleeding
within 48 hours After Onset of Index Major Bleeding Event using
sIPTW Propensity Score Weighting (Major Bleeding Patients)

| | Phase 3 | | PLATO | |
|---|---|---|---|---|
| | Bentracimab | | Control | |
| | (N = 68) | | (N = 134) | |
| Required Surgery within 48 Hours, | | | | |
| n (%) | | | | |
| Yes | 2 | (2.9) | 22 | (16.4) |
| No | 66 | (97.1) | 112 | (83.6) |

| | | |
|---|---|---|
| Estimated Risk of Surgery | 4.8 | 13.0 |
| 95% CI | (1.6, 13.5) | (7.8, 20.9) |
| Risk Difference (Bentracimab − | −8.2 | |
| Control) | | |
| 95% CI | (−16.1, −0.4) | |
| p-value | 0.0385 | |
| Risk Ratio (Bentracimab/Control) | 0.366 | |
| 95% CI | (−0.051, 0.783) | |
| p-value | 0.0029 | |

The Major Bleeding group included patients with procedure-related major bleeding and all patients with spontaneous major bleeding. Subgroup analysis of procedure-related major bleeding patients (Table 46) and spontaneous major bleeding patients (Table 47), which included ICH spontaneous major bleeding patients and non-ICH spontaneous major bleeding patients was performed. In the procedure-related major bleeding group, reduction in rate of requiring surgery to treat bleeding within 48 hours after the index major bleeding event compared to PLATO controls was observed (risk ratio: 0.602, 95% CI: (−0.167, 1.370)), though the sample size was underpowered to show statistical significance. In the spontaneous major bleeding group, the risk ratio of 0.202 (95% CI: (−0.232, 0.636)) indicates a statistically significant reduction in risk of requiring surgery to treat bleeding in bentracimab-treated patients, p-value=0.0003.

TABLE 46
Analysis of Proportion of Patients Requiring Surgery
to Treat Bleeding within 48 hours After Onset of Index
Major Bleeding Event using sIPTW Propensity Score Weighting
(Procedure-related Major Bleeding Patients)

| | |
|---|---|
| Phase 3 | PLATO |
| Bentracimab | Control |
| (N = 21) | (N = 82) |

| | | | |
|---|---|---|---|
| Required Surgery within 48 Hours, | | | |
| n (%) | | | |
| Yes | 1 | (4.8) | 13 (15.9) |
| No | 20 | (95.2) | 69 (84.1) |

| | | |
|---|---|---|
| Estimated Risk of Surgery | 9.1 | 15.1 |
| 95% CI | (2.8, 26.0) | (8.3, 25.9) |
| Risk Difference (Bentracimab − | −6.0 | |
| Control) | | |
| 95% CI | (−19.6, 7.5) | |
| p-value | 0.3831 | |
| Risk Ratio (Bentracimab/Control) | 0.602 | |
| 95% CI | (−0.167, 1.370) | |
| p-value | 0.3099 | |

TABLE 47
Analysis of Proportion of Patients Requiring Surgery
to Treat Bleeding within 48 hours After Onset of
Index Major Bleeding Event using sIPTW Propensity
Score Weighting (Spontaneous Major Bleeding Patients)

| | Phase 3 | | PLATO | |
|---|---|---|---|---|
| | Bentracimab | | Control | |
| | (N = 47) | | (N = 52) | |
| Required Surgery within 48 Hours, | | | | |
| n (%) | | | | |
| Yes | 1 | (2.1) | 9 | (17.3) |
| No | 46 | (97.9) | 43 | (82.7) |

| | | |
|---|---|---|
| Estimated Risk of Surgery | 2.5 | 12.1 |
| 95% CI | (0.3, 17.0) | (6.2, 22.4) |
| Risk Difference (Bentracimab − | −9.7 | |
| Control) | | |
| 95% CI | (−19.0, −0.4) | |
| p-value | 0.0419 | |
| Risk Ratio (Bentracimab/Control) | 0.202 | |
| 95% CI | (−0.232, 0.636) | |
| p-value | 0.0003 | |

### Bentracimab has a Favorable Safety Profile

Phase 3 patients treated with bentracimab had comparable or lower incidence of treatment-emergent serious adverse events (SAE), compared to PLATO control in both urgent surgery and major bleeding patient populations (Tables 48-50). Specifically, only 0.5% of SAEs (95% CI (0.1, 2.7)) led to study drug discontinuation. This indicates that bentracimab is safe and well-tolerated.

TABLE 48
Serious Adverse Events within 38 Days after Start of Urgent Surgery
or Major Bleeding (Urgent Surgery and Major Bleeding Patients)

| | Phase 3 Bentracimab | | | PLATO Control | | |
|---|---|---|---|---|---|---|
| | (N = 209) | | | (N = 374) | | |
| | n (%) | | 95% CI | n (%) | | 95% CI |
| Any SAEs | 65 | (31.1) | (25.2, 37.7) | 179 | (47.9) | (42.8, 52.9) |
| Any Severe SAEs | 39 | (18.7) | (14.0, 24.5) | 112 | (29.9) | (25.5, 34.8) |
| Any SAEs Leading to Study Drug | 1 | (0.5) | (0.1, 2.7) | 35 | (9.4) | (6.8, 12.7) |
| Discontinuation | | | | | | |
| Any SAEs Leading to Death | 9 | (4.3) | (2.3, 8.0) | 29 | (7.8) | (5.5, 10.9) |

TABLE 49
Serious Adverse Events within 38 Days after Start
of Urgent Surgery (Urgent Surgery Patients)

| | Phase 3 Bentracimab | | | PLATO Control | | |
|---|---|---|---|---|---|---|
| | (N = 141) | | | (N = 240) | | |
| | n (%) | | 95% CI | n (%) | | 95% CI |
| Any SAEs | 39 | (27.7) | (20.9, 35.6) | 93 | (38.8) | (32.8, 45.0) |
| Any Severe SAEs | 20 | (14.2) | (9.4, 20.9) | 60 | (25.0) | (19.9, 30.8) |
| Any SAEs Leading to Study Drug | 1 | (0.7) | (0.1, 3.9) | 17 | (7.1) | (4.5, 11.0) |
| Discontinuation | | | | | | |
| Any SAEs Leading to Death | 4 | (2.8) | (1.1, 7.1) | 16 | (6.7) | (4.1, 10.6) |

TABLE 50
Serious Adverse Events within 38 Days after Start
of Major Bleeding (Major Bleeding Patients)

| | Phase 3 Bentracimab | | | PLATO Control | | |
|---|---|---|---|---|---|---|
| | (N = 68) | | | (N = 134) | | |
| | n (%) | | 95% CI | n (%) | | 95% CI |
| Any SAEs | 26 | (38.2) | (27.6, 50.1) | 86 | (64.2) | (55.8, 71.8) |
| Any Severe SAEs | 19 | (27.9) | (18.7, 39.6) | 52 | (38.8) | (31.0, 47.3) |
| Any SAEs Leading to Study Drug | 0 | (0.0) | (0.0, 5.3) | 18 | (13.4) | (8.7, 20.2) |
| Discontinuation | | | | | | |
| Any SAEs Leading to Death | 5 | (7.4) | (3.2, 16.1) | 13 | (9.7) | (5.8, 15.9) |

What is claimed is:

1. A method of reversing ticagrelor-enhanced bleeding in a patient comprising administering bentracimab to the patient during surgery or during an invasive procedure.

2. The method of claim 1, wherein the patient requires nondeferrable surgery or an invasive procedure or is experiencing major bleeding.

3. The method of claim 1, wherein the bentracimab is administered during surgery.

4. The method of claim 3, wherein the surgery is coronary artery bypass graft (CABG), cardiac surgery, gastrointestinal surgery, coronary artery procedure, repair of aortic dissection, or cardiac valve replacement.

5. The method of claim 1, wherein the bentracimab is administered during an invasive procedure.

6. The method of claim 5, wherein the invasive procedure is percutaneous cardiac intervention or another cardiac or vascular procedure.

7. The method of claim 1, wherein the patient is elderly.

8. The method of claim 1, wherein the patient has an underlying cardiac or pulmonary disease, has limited cardiac reserve, or has a metabolic disease.

9. The method of claim 1, wherein the bentracimab is administered intravenously.

10. The method of claim 1, wherein about 18 g, about 24 g, or about 36 g of bentracimab is administered.

11. The method of claim 1, wherein the bentracimab administered over about 10 min to about 36 hours.

12. The method of claim 1, wherein the bentracimab is administered according to a schedule comprising about 6 g over about 10 minutes, followed by about 6 g over about 4 hours, and followed by about 6 g over about 12 hours.

13. The method of claim 1, wherein the bentracimab is administered according to a schedule comprising about 6 g over about 10 minutes, followed by about 6 g over about 4 hours, followed by about 6 g over about 12 hours, and followed by about 6 g over about 12 hours.

14. The method of claim 1, wherein the bentracimab is administered according to a schedule comprising about 12 g over about 10 minutes, followed by about 12 g over about 6 hours, and followed by about 12 g over about 12 hours.

15. The method of claim 14, wherein the has been treated with a moderate or strong CYP3A inhibitor.

16. The method of claim 1, wherein the bentracimab is administered at a concentration of about 100 mg/mL.

17. The method of claim 1, wherein administering the antibody or antigen-binding fragment thereof to the patient results in an increase in PRU in the patient.

18. The method of claim 1, wherein platelet function returns to normal within 5-10 minutes of administration of bentracimab.

19. A method of reversing ticagrelor-enhanced bleeding in a patient that requires nondeferrable surgery or an invasive procedure or a patient that is experiencing major bleeding, comprising:
performing surgery or an invasive procedure on the patient; and
administering bentracimab to the patient during the surgery or during an invasive procedure.

20. The method of claim 19, wherein the surgery is coronary artery bypass graft (CABG), cardiac surgery, gastrointestinal surgery, coronary artery procedure, repair of aortic dissection, or cardiac valve replacement.

21. The method of claim 19, wherein the invasive procedure is percutaneous cardiac intervention or another cardiac or vascular procedure.

22. The method of claim 19, wherein the patient is elderly.

23. The method of claim 19, wherein the patient has an underlying cardiac or pulmonary disease, has limited cardiac reserve, or has a metabolic disease.

24. The method of claim 19, wherein the bentracimab is administered intravenously.

25. The method of claim 19, wherein the bentracimab is administered at a concentration of about 100 mg/mL.

26. The method of claim 19, wherein administering the antibody or antigen-binding fragment thereof to the patient results in an increase in minimum percent inhibition of P2Y₁₂ reaction units (PRU) in the patient.

27. The method of claim 19, wherein the patient has uncontrolled major or life-threatening bleeding and administering the bentracimab to the patient results in a minimum percent inhibition of P2Y₁₂ reaction units (PRU) over 4 hours after starting administration of between about −78 and about −56 in the patient.

28. The method of claim 19, wherein the patient requires surgery or invasive procedure and administering the bentracimab to the patient results in a minimum percent inhibition of P2Y₁₂ reaction units (PRU) over 4 hours after starting administration of between about −78 and about −68 in the patient.

29. The method of claim 19, wherein platelet function returns to normal within 5-10 minutes of administration of bentracimab.

30. The method of claim 19, further comprising restarting ticagrelor when treatment with Bentracimab is complete.


SEQUENCE LISTING (8 sequences)

SEQ ID NO: 1 - protein, 129 aa, synthetic construct
QVQLQESGAEVKKPGSSVRVSCKASGGTFDSYSIHWVRQAPGQGLEWMGGIIPAFGTLSSAQDFQARVTISADKSTSTAYMELSGLRSEDTAVYYCARGSFDYYFWSASHPPNDALAIWGQGTLVTVSS

SEQ ID NO: 2 - protein, 110 aa, synthetic construct
QSVVTQPPSVSAAPGQKVTISCSGSNSDIGNNYVSWYQQLPGTAPKLLIYDNNKRPSGIPDRFSGSKSGTSATLAITGLQAGDEADYYCGTWLYDRAVGLFGGGTKVTVL

SEQ ID NO: 3 - protein, 5 aa, synthetic construct
SYSIH

SEQ ID NO: 4 - protein, 17 aa, synthetic construct
GIIPAFGTLSSAQDFQA

SEQ ID NO: 5 - protein, 20 aa, synthetic construct
GSFDYYFWSASHPPNDALAI

SEQ ID NO: 6 - protein, 13 aa, synthetic construct
SGSNSDIGNNYVS

SEQ ID NO: 7 - protein, 7 aa, synthetic construct
DNNKRPS

SEQ ID NO: 8 - protein, 11 aa, synthetic construct
GTWLYDRAVGL